(19) United States
(12) Patent Application Publication
(10) Pub. No.: US 2024/0190871 A1
(43) Pub. Date: Jun. 13, 2024

(54) KRASG12D INHIBITOR, PREPARATION METHOD THEREFOR AND APPLICATION THEREOF

(71) Applicant: YA THERAPEUTICS INC, Shanghai (CN)

(72) Inventors: Yonghong LIANG, Shanghai (CN); Zhiyong XU, Shanghai (CN)

(21) Appl. No.: 18/547,515

(22) PCT Filed: Feb. 9, 2022
(86) PCT No.: PCT/CN2022/075649

§ 371 Date: Aug. 23, 2023

(30) Foreign Application Priority Data
Mar. 15, 2021 (CN) 202110277549.5
Apr. 29, 2021 (CN) 202110476710.1

(51) Int. Cl. C07D 471/04; A61P 35/00; C07D 519/00

(52) U.S. Cl. CPC C07D 471/04; A61P 35/00; C07D 519/00

(57) ABSTRACT

KRAS^{G12D} mutant protein inhibitors, as shown by formula (I), a composition containing the inhibitor and pharmaceutically acceptable salt, syntheses, intermediates, formulations, and the use thereof. The compounds of the invention have good activity and safety in inhibiting tumor growth.

### CROSS-REFERENCE TO RELATED APPLICATIONS

This application claims the priority of Chinese patent application CN2021102775495 with an application date of Mar. 15, 2021 and Chinese patent application CN2021104767101 with an application date of Apr. 29, 2021. This application cites the full text of the above Chinese patent application.

### BACKGROUND OF THE INVENTION

The invention belongs to the field of drug synthesis, and in particular relates to a novel KRASG12D inhibitor and a preparation method and application thereof.

The present invention generally relates to novel compounds and methods for their preparation and use as inhibitors of KRASG¹²D, eg for the treatment of cancer.

RAS represents a group of closely related monomeric globular proteins of 189 amino acids (molecular weight 21 kDa) that associate with the plasma membrane and bind GDP or GTPoRAS as a molecular switch. When the RAS contains bound GDP, it is in a quiescent or off state and is in an “inactive state”. RAS is induced to convert its bound GDP to GTP in response to exposure of cells to certain growth-promoting stimuli. Upon binding to GTP, RAS is “turned on” and is able to interact with and activate other proteins (its “downstream targets”). The RAS protein itself has a very low intrinsic ability to hydrolyze GTP back to GDP, thus shutting itself down. Turning off RAS requires extrinsic proteins called GTPase-activating proteins (GAPs), which interact with RAS and greatly accelerate the conversion of GTP to GDP. Any mutation in RAS that affects its ability to interact with GAP or convert GTP back to GDP will result in prolonged activation of the protein, leading to prolonged signaling in the cell to continue growing and dividing. Because these signals cause cells to grow and divide, overactive RAS signaling may ultimately lead to cancer.

Structurally, RAS proteins contain a G domain that is responsible for the enzymatic activity of RAS—the binding and hydrolysis of guanine nucleoproteins (GTPase reaction). It also contains a C-terminal extension called the CAAX box, which allows for post-translational modifications and is responsible for targeting proteins to the membrane. The G domain is approximately 21-25 kDa in size and contains a phosphate-binding loop (P-loop). The P-loop is the nucleotide binding pocket in the protein, which is a rigid part of a domain with conserved amino acid residues ((glycine 12, threonine 26, and lysine 16)), which is important for nucleotide binding and Hydrolysis is critical. The G domain also contains the so-called Switch I (residues 30-40) and Switch II (residues 60-76) regions, both of which are dynamic parts of the protein due to their ability to switch between resting and loaded states, It is often referred to as the ‘spring-loaded’ mechanism. The key interaction is the hydrogen bond formed by threonine 35 and glycine 60, with the Y-phosphate of GTP, which keeps the Switch1 and Switch2 regions in their active conformations, respectively. GTP hydrolysis and After releasing the phosphate, the two relax to the inactive GDP conformation.

The best-known members of the RAS subfamily are HRAS, KRAS, and NRAS, mainly because of their association with multiple types of cancer. Mutations in any of the three major isoforms of RAS (HRAS, NRAS, or KRAS) genes are the most common in human tumorigenesis. About 30% of human tumors were found to carry mutations in the RAS gene. Notably, KRAS mutations were detected in 25-30% of tumors. In contrast, oncogenic mutations occurred at much lower rates in NRAS and HRAS family members (8% and 3%, respectively). The most common KRAS mutations were found at residues G12 and G13 of the P loop and at residue Q61. G12C and G12D are frequent mutations of the KRAS gene (glycine 12 to cysteine and glycine 12 to aspartate).

As cutting-edge targets, KRASG12C and KRASG12D mutant proteins have received extensive attention. Among them, many KRASG12C inhibitors are in clinical trials, such as: Amgen's AMG-510 (WO2018217651A1) and Mirati Pharmaceuticals' MRTX-849 (WO2019099524A1). However, there is currently no corresponding targeted drug for KRASG12D mutant protein. Recently, a candidate KRASG12D inhibitor MRTX-1133 (WO2021041671A1) developed by Mirati Pharmaceuticals is in preclinical stage. The present invention satisfies this need and provides other related advantages.

### SUMMARY

A compound with general formula (1), its stereoisomer, pharmaceutically acceptable salt, polymorph or isomer, wherein the structure of the compound shown in general formula (I) is as follows:

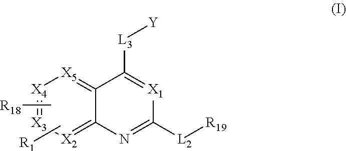

Wherein,
Each L₁ at each occurrence is independently selected from bond, —C₁₋₄alkyl-, —CR₈R₉—, —C₁₋₂alkyl(R₈)(OH)—, —C(O)—, —CR₈R₉O—, —OCR₈R₉—, —SCR₈R₉—, —CR₈R₉S—, —NR₈—, —NR₈C(O)—, —C(O)NR₈—, —NR₈C(O)NR₉—, —CF₂—, —O—, —S—, —S(O)ₘ—, —NR₈S(O)ₘ—, —S(O)ₘNR₈—;
Each R₁ at each occurrence is independently selected from phenyl, naphthyl, 5-membered heteroaryl, 6-membered heteroaryl, 7-membered heteroaryl, 8-membered heteroaryl, 9-membered heteroaryl, or 10-membered heteroaryl heteroaryl, each heteroaryl independently at each occurrence containing 1, 2, 3 or 4 heteroatoms selected from N, O, or S; each R1 at each occurrence independently and optionally substituted or unsubstituted with 1, 2, 3, 4, 5 or 6 R₂₀;
Each R₂₀ at each occurrence is independently selected from deuterium, halogen, oxo, —C₁₋₆ alkyl, —C₂₋₆ alkenyl, —C₂-alkynyl, —C₁₋₆ alkylene-(halogen)₁₋₃, C₁₋₆ heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene-(halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR6C(═O)R₇, —S(O)₂NR₆R₇ or —C₃₋₆ carbocyclyl; each R₂₀ is independently and optionally substituted or unsubstituted with 1, 2, 3, 4, 5 or 6 substituents selected from deuterium, halogen, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇.
Each X₁, X₂, X₃, X₄, X₅ is independently selected from N, CR₂₁ at each occurrence;
Each R₂₁ is independently selected from H, D, cyano, halogen, C₁₋₆ alkyl, COOH, NHCOH, CONH₂, OH or —NH₂;
Each R₁₈ is independently selected from H, D, cyano, halogen, C₁₋₆ alkyl, COOH, NHCOH, CONH₂, OH or —NH₂;
Each L₂ is independently selected at each occurrence from bond, OC₀₋₆ alkyl, NC₀₋₆ alkyl, C₁₋₆ alkyl, COC₀₋₆ alkyl or SC₀₋₆ alkyl;
Each R₁₉ is independently selected from

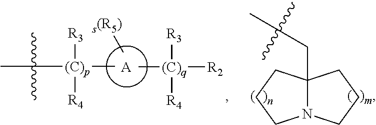

—C₁₋₆ alkyl, —C₂₋₆ alkenyl, —C₂-alkynyl, —C₁₋₆ alkylene-(halogen)₁₋₃, C₁₋₆ heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene Base-(halogen)₁₋₃, —NR₆R₇, —C₁₋₆ alkylene-NR₆R₇, —C(═O)R₆, —C(═O)OR₆, —OC(═O)R₆, —C(═O) NR₆R₇, —NR₆C(═O)R₇, —S(O)₂NR₆R₇, —C₃₋₆ carbocyclyl, 3-8 membered heterocycle; 3-8 membered heterocycle independently at each occurrence contains 1, 2, 3 or 4 heteroatoms selected from N, O, or S; cach R₁₉ is independently and optionally are substituted or unsubstituted with 1, 2, 3, 4, 5 or 6 substituents selected from deuterium, halogen, —C₁₋₆ alkane base, —C₁₋₆ alkoxy, oxo, —OR₆, —NR₆R₇, —CN, —C(═O)R₆, —C(═O)OR₆, —OC(═O)R₆, —C(═O) Substituents of NR₆R₇, —NR₆C(═O)R; or —S(O)₂NR₆R₇.

Each ring A is a C₃₋₁₀ carbocyclic ring, and the

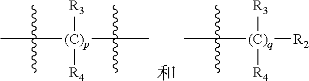

can be attached to the same carbon atom or to a different atom of the ring A;
Each R₂ is —OR₆, —NR₆R₇, —SR₆, —S(═O)R₆, —S(═O)₂R₆, 5-10 membered heteroaryl or 3-10 membered heterocyclyl, each heterocyclyl and heteroaryl at each occurrence independently contain 1, 2, 3 or 4 heteroatoms selected from N, O, S, S═O or S(═O)₂, each R₂ at each occurrence independently and optionally substituted or unsubstituted with 1, 2, 3, 4, 5 or 6 R₂₂;
Each R₃ and R₄ at each occurrence is independently selected from deuterium, hydrogen, halogen, —C₁₋₆ alkyl, —C₂₋₆ alkenyl, —C₂₋₆ alkynyl, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇ or —C₃₋₁₀ carbocyclyl, each heterocyclyl and heteroaryl at each occurrence independently contain 1, 2, 3 or 4 heteroatonsselected from N, O, S, S═O or S(═O)₂; each R₃ and R₄ at each occurrence is independently optionally substituted or unsubstituted with 1, 2, 3, 4, 5 or 6 substituents selected from deuterium, halogen, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇;
Each R₅ at each occurrence is independently selected from deuterium, halogen, oxo, —C₁₋₆ alkyl, —C₁₋₆ alkylene-(halogen)1-3, C₁₋₆ heteroalkyl, —CN , —OR₆, —C₁₋₆ alkylene-(OR₆)₁₋₃, —O—C1₋₆ alkylene-(halogen)₁₋₃, —SR₆, —S—C₁₋₆ alkylene-(Halogen)₁₋₃, —NR₆R₇, —C₁₋₆ alkylene —NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl, each heterocyclyl and heteroaryl independently at each occurrence contains 1, 2, 3 or 4 options A heteroatom from N, O, S, S═O or S(═O)₂; each R₃ and R₄ at cach occurrence independently and optionally substituted or unsubstituted with 1, 2, 3, 4, 5 or 6 substituents selected from deuterium, halogen, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇;
Each R₆ and R₇ at each occurrence is independently selected from hydrogen or —C₁₋₆ alkyl, each R₆ and R₇ is independently optionally substituted with 1, 2, 3, 4, 5 or 6 R₂₂ or not or R₇ and R₇ together with the N atom to which they are connected together form a 3-10-membered heterocycle, and the 3-10-membered heterocycle may further comprise 1, 2, 3 or 4 selected from N, O, S, A heteroatom of S(═O) or S(═O)₂, and the 3-10 membered heterocycle is independently optionally substituted or unsubstituted by 1, 2, 3, 4, 5 or 6 R₂₂;
Each R₂₂ at each occurrence is independently selected from deuterium, halogen, oxo, —C₁₋₆ alkyl, —C₁₋₆ alkylene-(halogen)₁₋₃, —C₁₋₆ heteroalkyl, —CN, —O—C₁₋₆ alkyl, —C₁₋₆ alkylene-(O—C₁₋₆ alkyl₁₋₃, —O—C₁₋₆ alkylene-(halogen)₁₋₃, —S—C₁₋₆ alkyl, —S—C₁₋₆ alkylene-(halogen)₁₋₃, —N—C₁₋₆ alkyl-C₁₋₆ alkyl, —C₁₋₆ alkylene-NC₁₋₆ alkyl base C₁₋₆ alkyl, —C(═O)C₁₋₆ alkyl, —C(═O)OC₁₋₆ alkyl, —OC(═O)C₁₋₆ alkyl, —C(═O)NC₁₋₆ alkyl C₁₋₆ alkyl, —NC₁₋₆ alkyl C(═O)C₁₋₆ alkyl, —S(O)₂NC₁₋₆ alkyl C₁₋₆ alkyl or —C₃₋₆ carbocyclyl;
s is selected from 0, 1, 2, 3, 4, 5 or 6;
p is selected from 0, 1, 2, 3, 4, 5 or 6;
q is selected from 0, 1, 2, 3, 4, 5 or 6;
m is selected from 1, 2, 3;
n is selected from 1, 2, 3
Y is absent or selected from 3-8 membered cycloalkyl, 3-8 membered heterocycloalkyl, 5-12 membered fused alkyl, 5-12 membered fused heterocyclyl, 5-12 membered spirocyclyl, 5-12 membered Membered spiroheterocyclyl, aryl or heteroaryl, each heterocycloalkyl, fused heterocyclyl, spiroheterocyclyl, heteroaryl at each occurrence independently contains 1, 2, 3 or 4 heteroatoms selected from N, O, or S, wherein the cycloalkyl, heterocycloalkyl, spiro, fused ring, fused heterocyclyl, spiroheterocyclyl, aryl, or heteroaryl group is optionally replaced by one or more G¹s;
G¹ and G² are each independently selected from deuterium, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR₈, —OC(O)NR₈R₉, —C(O)OR₈, —C(O)NR₈R₉, —C(O)R₈, —NR₈R₉, —NR₈C(O)R₉, —NR₈C(O)NR₉R₁₀, —S(O)ᵢR₈ or —NR₈S(O)ᵢR₉, wherein the alkyl, alkenyl, alkynyl, cycloalkyl, heterocycloalkyl, aryl, heteroaryl is optionally substituent with 1 or more deuterium, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀-aryl, 5-10-membered heteroaryl, —OR₁₁, —OC(O)NR₁₁R₁₂, —C(O)OR₁₁, —C(O)NR₁₁R₁₂, —C(O)R₁₁, —NR₁₁R₁₂, —NR1₁C(O)R₁₂, —NR₁₁C(O)NR₁₂R₁₃, —S(O)ᵢR₁₁ or —NR₁₁S(O)ᵢR₁₂;
R₈, R₉, R₁₀, R₁₁, R₁₂ and R₁₃ are each independently selected from hydrogen, deuterium, cyano, halogen, C₁₋₆ alkyl, C₃₋₈ cycloalkyl or 3-8 membered monocyclic heterocyclyl, monocyclic heterocyclyl aryl or phenyl;
and i is 1 or 2.

In some embodiments, each R₁ at each occurrence is independently selected from phenyl, naphthyl, 5-membered heteroaryl, 6-membered heteroaryl, 7-membered heteroaryl, 8-membered heteroaryl, 9-membered Heteroaryl or 10-membered heteroaryl, each heteroaryl independently at each occurrence containing 1, 2, 3 or 4 heteroatoms selected from N, O, or S; each R₁ at each occurrence is independently and optionally substituted or unsubstituted with 1, 2, 3, 4, 5 or 6 R₂₀;
In some embodiments, each R₁ at each occurrence is independently selected from phenyl, naphthyl, pyridyl, indolyl, indazolyl, benzofuranyl, benzothienyl, quinolyl, iso Quinolinyl, each R₁ at each occurrence is independently and optionally substituted or unsubstituted with 1, 2, 3, 4, 5 or 6 R₁₂; each R₁ at each occurrence independently optionally substituted or unsubstituted with 1, 2, 3, 4, 5 or 6 R₂₀;
In some embodiments, each R₁ is selected from:

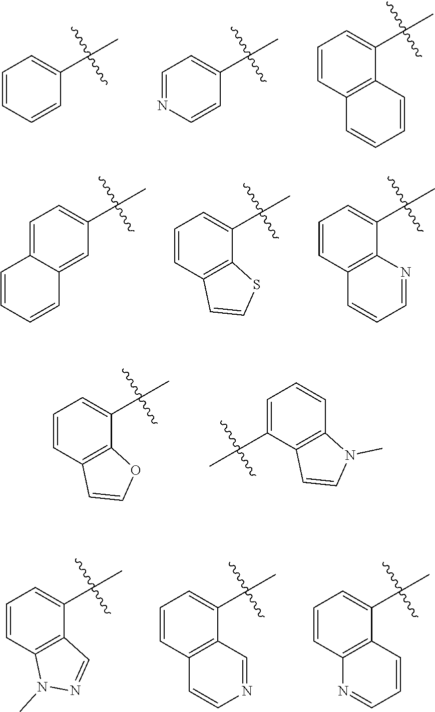

In some embodiments, each R₁ is independently and optionally substituted or unsubstituted at each occurrence with 1, 2, 3, 4, 5, or 6 R₂₀;
In some embodiments, each R₂₀ at each occurrence is independently selected from deuterium, halogen, oxo, —C₁₋₆ alkyl, —C₁₋₆ alkylene-(halogen)₁₋₃, C₁₋₆ Heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene-(halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl; each R₁₂ is independently and optionally substituted or unsubstituted with 1, 2, 3, 4, 5 or 6 substituents selected from deuterium, halogen, —C₁₋₆ alkyl, —C₁₋₆ alkoxy, oxo, —OR₆, —NR₆R₇, —CN, —C(═O)R₆, —C(═O)OR₆, Substituents of —OC(═O)R₆, —C(═O)NR₆R₇, —NR₆C(═O)R₇ or —S(O)₂NR₆R₇.;
In some embodiments, R₆ and R₇ in each R₂₀ are independently selected at each occurrence from hydrogen, deuterium, or —C₁₋₃ alkyl;
In some embodiments, each R₂₀ at each occurrence is independently selected from -deuterium, —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)2NH(CH₃), —S(O)₂N(CH₃)₂, 3-membered carbocyclyl, 4-membered carbocyclyl, 5-membered carbocyclyl or 6-membered carbocyclyl; each R₂₀ is independently and optionally substituted or unsubstituted with 1, 2, 3, 4, 5 or 6 substituents selected from -deuterium, —F, —Cl, —Br, methyl, ethyl, propyl , isopropyl, methoxy, ethoxy, propoxy, isopropoxy, oxo, _OH, —NH₂, —NHCH₃, _N(CH₃)₂, —CN, —C(═O)CH₃, _C(═O)OO, —OC(═O)O, —C(═O)NH₂, —C(═O)NH(CH₃), —C(═O)N(CH₃)₂, —NHC(═O)CH₃, —N(CH₃) of C(═O)CH₃, —S(O)₂NH₂, —S(O)₂NH(CH₃) or —S(O)₂N(CH₃)₂.

In some embodiments, each R₁ is selected from:

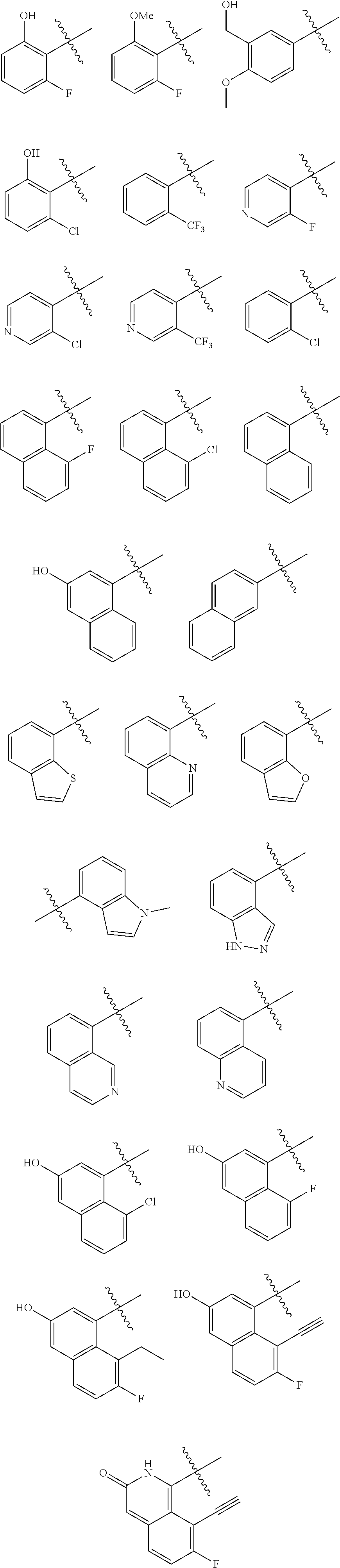

In some embodiments, each ring A is absent, a 3-membered carbocycle, a 4-membered carbocycle, a 5-membered carbocycle, or a 6-membered carbocycle, and the

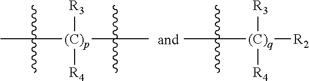

can be attached to the same ring A on a same carbon atom or on a different atom; each R₂ at each occurrence is independently selected from —NR₆R₇ or a 3-6 membered heterocyclyl, each heterocyclyl at each occurrence independently contains 1 heteroatom selected from N, each R₂ at each occurrence is independently and optionally substituted or unsubstituted with 1, 2, 3, 4, 5 or 6 R₂₀;
In some embodiments, R₆ and R₇ in each R₂ at each occurrence are independently selected from bydrogen, deuterium, methyl, ethyl, propyl, or isopropyl; or R6 and R₇ in R₂ are taken together The connected N atoms together form a 3-6 membered heterocycle, the 3-6 membered heterocycle may further comprise 1 heteroatom selected from N, and the 3-6 membered heterocycle is independently and optionally substituted or unsubstituted with 1, 2, 3, 4, 5 or 6 R₂₀;
In some embodiments, each R₂ is at each occurrence independently selected from —NH₂, —N(CH₃)₂, —N(CH₃)(CH₂CH₃), —N(CH₂CH₃)₂,

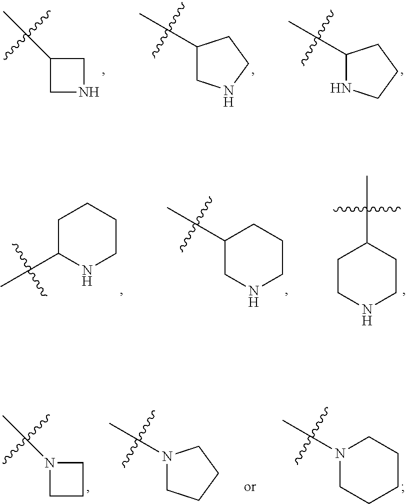

each R₂ is independently and optionally substituted or unsubstituted with 1, 2, 3, 4, 5 or 6 R₂₀;
In some embodiments, each R₂ at each occurrence is independently selected from —NH₂, —N(CH₃)₂, —N(CH₃)(CH₂CH₃), —N(CH₂CH₃)₂,

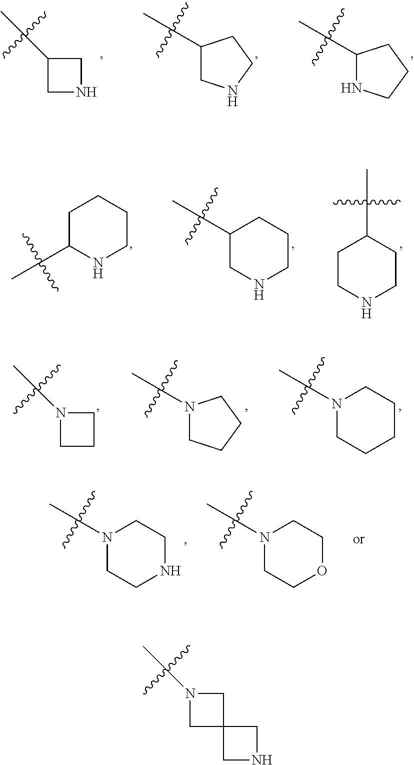

each R₂ is independently and optionally substituted or unsubstituted with 1, 2, 3, 4, 5 or 6 R₂₀;
In some embodiments, each R₂₀ at each occurrence is independently selected from deuterium, halogen, oxo, —C₁₋₆ alkyl, —C₁₋₆ alkylene-(halogen)₁₋₃, C₁₋₆ Heteroalkyl, —CN, —OR₆, —C₁₋₆ alkylene-(OR₆)₁₋₃, —O—C₁₋₆ alkylene-(halogen)₁₋₃, —SR₆, —S-C₁₋₆ alkylene-(halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl; each R₁₂ is independently and optionally substituted or unsubstituted with 1, 2, 3, 4, 5 or 6 substituents selected from deuterium, halogen, —C₁₋₆ alkyl, —C₁₋₆ alkoxy, oxo, —OR₆, —NR₆R₇, —CN, —C(═O)R₆, —C(═O)OR₆, Substituents of —OC(═O)R₆, —C(═O)NR₆R₇, —NR₆C(═O)R₇ or —S(O)₂NR₆R₇
In some embodiments, each R₂₀ at each occurrence is independently selected from -deuterium, —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH2CH2CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocyclyl, 5-membered carbocyclyl or 6-membered carbocyclyl; each R₂₀ is independently and optionally substituted or unsubstituted with 1, 2, 3, 4, 5 or 6 substituents selected from -deuterium, —F, —Cl, —Br, methyl, ethyl, propyl, isopropyl, methoxy, ethoxy, propoxy, isopropoxy, oxo, —OH, —NH₂, —NHCH₃, _N(CH₃)₂, —CN, —C(═O)CH₃, —C(═O)OO, —OC(═O)O, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

In some embodiments, each R₃ and R₄ at each occurrence is independently selected from deuterium, hydrogen, halogen, —C₁₋₆ alkyl, —C₂₋₆ alkenyl, —C₂₋₆ alkynyl, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇ or —C₃₋₁₀ carbocyclyl, each heterocyclyl and heteroaryl at each occurrence independently contain 1, 2, 3 or 4 heteroatoms selected from N, O, S, S═O or S(═O)₂; each R₃ and R₄ at cach occurrence is independently and optionally substituted or unsubstituted with 1, 2, 3, 4, 5 or 6 atoms selected from deuterium, halogen, oxo, —C₁₋₆ alkyl, —C₁₋₆ alkoxy, oxo, —OR₆, —NRR, —CN, —C(═O)R, —C(═O)OR, —OC(═O)R, Substituents of —C(═O)NR₆R₇, —NR₆C(═O)R₇ or —S(O)₂NR₆R₇;
In some embodiments, R₆ and R₇ in each of R₃ and R₄ at each occurrence are independently selected from hydrogen, deuterium, or —C₁₋₃ alkyl;
In some embodiments, each R₃ and R₄ at each occurrence is independently selected from hydrogen, —F, —Cl, —Br, methyl, ethyl, propyl, isopropyl, vinyl, propenyl, isopropenyl, ethynyl, propynyl, oxo, —OH, —OCH₃, —OCH₂CH₃, —OCH₂CH₂CH₃, —OCH(CH₃)₂, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocycle group, 5-membered carbocyclyl, or 6-membered carbocyclyl; each R₅ or R₆ is independently and optionally substituted or unsubstituted with 1, 2, 3, 4, 5 or 6 substituents selected from —F, —Cl, —Br, oxo, methyl, ethyl, propyl, isopropyl, —OH, OCH₃, —OCH₂CH₃, —OCH₂CH₂CH₃, —OCH(CH₃)₂, —NH₂, —N(CH₃)₂, —CN, —C(═O)CH₃, —OC(═O)CH₃, —C(═O)NH₂, —C(═O)NH(CH₃), —C(═O)N(CH₃)₂, —NHC(═O)CH₃, Substituents of —N(CH₃)C(═O)CH₃, —S(O)₂NH₂, —S(O)₂NH(CH₃), —S(O)₂N(CH₃)₂.

In some embodiments, each R₅ at each occurrence is independently selected from deuterium, —F, —Cl, —Br, —C₁₋₃ alkyl, —C₁₋₃ alkylene-(halogen)₁₋₃, C₁₋₃ heteroalkyl, —C₂₋₃ alkenyl, —C₂₋₃ alkynyl, —CN, —OR6, —C₁₋₆ alkylene-(OR₆)₁₋₃, —O—C₁₋₆ Alkylene-(halogen)₁₋₃, —SR₆, —S—C₁₋₆ Alkylene-(halogen)₁₋₃, —NR₆R₇, —C₁₋₆ Alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl, each heterocyclyl and heteroaryl groups at each occurrence independently contain 1, 2, 3 or 4 heteroatoms selected from N, O, S, S═O or S(═O)₂; each R₃ and R₄ at each occurrence is independently and optionally substituted or unsubstituted with 1, 2, 3 or 4, 5 or 6 selected from deuterium, —F, —Cl, —Br, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —Substituents of NR₆C(═O)R₇ or —S(O)₂NR₆R₇.

In some embodiments, R₆ and R₇ in each R₅ at each occurrence are independently selected from hydrogen, deuterium, or —C₁₋₃ alkyl, or R₆ and R₇ in R₅ together with the N atom to which they are attached together form a 3-6 membered heterocycle, the 3-6 membered heterocycle may further comprise 1 heteroatom selected from N, and the 3-6 membered heterocycle heterocycles are independently and optionally contain 1, 2, 3, 4 heteroatoms selected from N, O or S;

In some embodiments, each R₅ at each occurrence is independently selected from deuterium, —F, —Cl, —Br, methyl, ethyl, propyl, isopropyl, vinyl, propenyl, isopropyl propenyl, ethynyl, propynyl, -methylene-(halogen)₁₋₃, -ethylene-(halogen)₁₋₃, -propylene-(halogen)₁₋₃, heteromethyl, Heteroethyl, heteropropyl, vinyl, propenyl, ethynyl, propynyl, oxo, —OR₆, -methylene-(OR₆)₁₋₃, -ethylene-(OR₆)₁₋₃, -propylene-(OR₆)₁₋₃, —O-methylene-(halogen)₁₋₃, —O-ethylene-(halogen)₁₋₃, —O-propylene-(halogen))₁₋₃, —NR₆R₇, -methylene-NR₆R₇, -ethylene-NR₆R₇, -propylene-NR₆R₇, —CN, —C(═O)R₆, —C(═O)OR₆, —OC(═O)R₆, —C(═O)NR₆R₇, —NR₆C(═O)R₇, —S(O)₂NR₆R₇, phenyl, naphthyl, 5-membered heteroaryl, 6-membered heteroaryl, 7-membered heteroaryl Aryl, 6-membered heteroaryl, 8-membered heteroaryl, 10-membered heteroaryl, 3-membered heterocyclyl, 4-membered heterocyclyl, 5-membered heterocyclyl, 6-membered heterocyclyl, 3-membered carbocyclyl , 4-membered carbocyclyl, 5-membered carbocyclyl or 6-membered carbocyclyl, each heterocyclyl and heteroaryl at each occurrence independently contain 1, 2, 3 or 4 heteroatoms selected from N, O or S; each R₇ at each occurrence is independently and optionally substituted or unsubstituted with 1, 2, 3, 4, 5 or 6 substituents selected from —F, —Cl, —Br, oxo, methyl, ethyl, propyl, isopropyl, —OR₆, —NR₆R₇, —CN, —C(═O)R₆, —C(═O)OR₆, —OC(═O)R₆, —C(═O)NR₆R₇, —NR₆C(═O)R₆, or —S(O)₂NR₆R₇;
In some embodiments, R₆ and R₇ in each R₅ at each occurrence are independently selected from hydrogen, deuterium, methyl, ethyl, propyl, isopropyl; or R₆ and R₇ in each R₅ together with the N atom to which they are attached together form

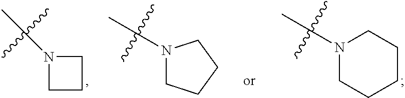 [SMILES: *N1CCC1 ; *N1CCCC1 ; *N1CCCCC1]

In some embodiments, each R₅ is independently selected at each occurrence from deuterium, —F, —Cl, —Br, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocyclyl, 5-membered carbocyclyl, or 6-membered Carbocyclyl; each R₂₀ is independently and optionally substituted or unsubstituted 1, 2, 3, 4, 5 or 6 substituents selected from -deuterium, —F, —Cl, —Br, methyl, ethyl, propyl, isopropyl base, methoxy, ethoxy, propoxy, isopropoxy, oxo, —OH, —NH₂, —NHCH₃, _N(CH₃)₂, —CN, —C(═O)CH₃, _C(═O)OO, —OOC(═O)O, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂;
In some embodiments, L₁-R₁₉ are selected from the following structures:

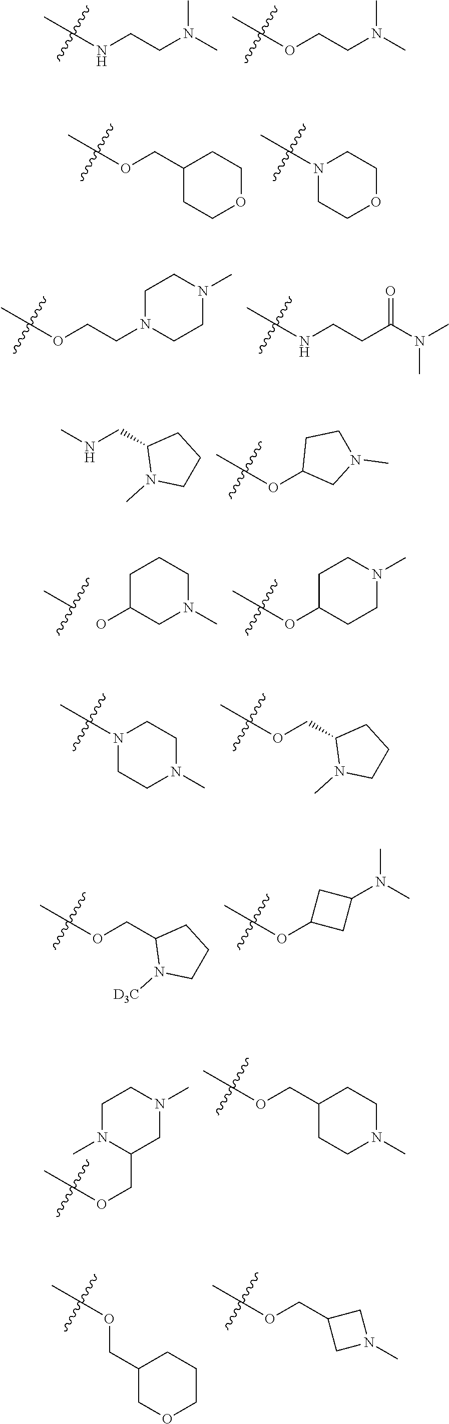

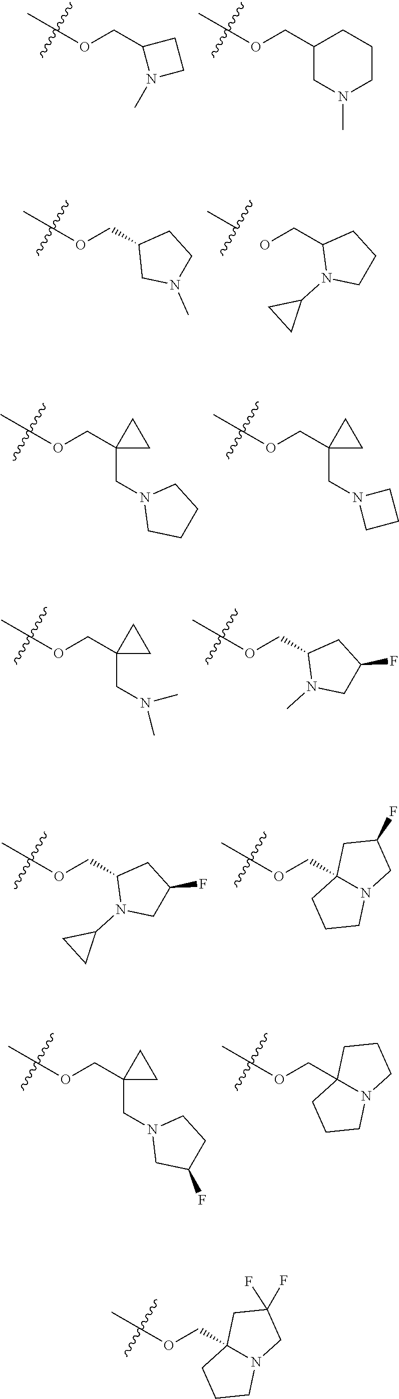

In some embodiments, the compound of formula (I) or an isomer, solvate or precursor thereof, or a pharmaceutically acceptable salt thereof is selected from the group consisting of the following compounds, isomers, solvates thereof or their precursors, or their pharmaceutically acceptable salts:

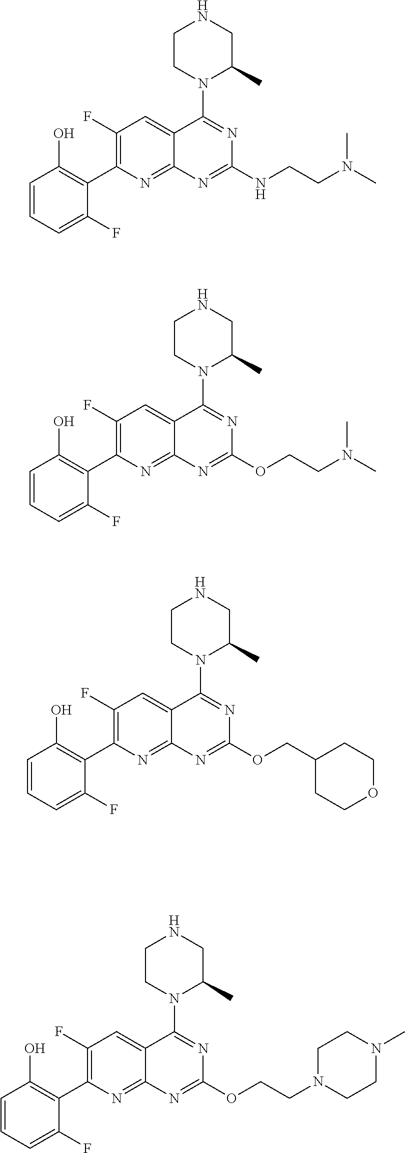 [SMILES: C[C@@H]1CNCCN1c1nc(NCCN(C)C)nc2nc(-c3c(O)cccc3F)c(F)cc12 ; C[C@@H]1CNCCN1c1nc(OCCN(C)C)nc2nc(-c3c(O)cccc3F)c(F)cc12 ; C[C@@H]1CNCCN1c1nc(OCC2CCOCC2)nc2nc(-c3c(O)cccc3F)c(F)cc12 ; C[C@@H]1CNCCN1c1nc(OCCN2CCN(C)CC2)nc2nc(-c3c(O)cccc3F)c(F)cc12]

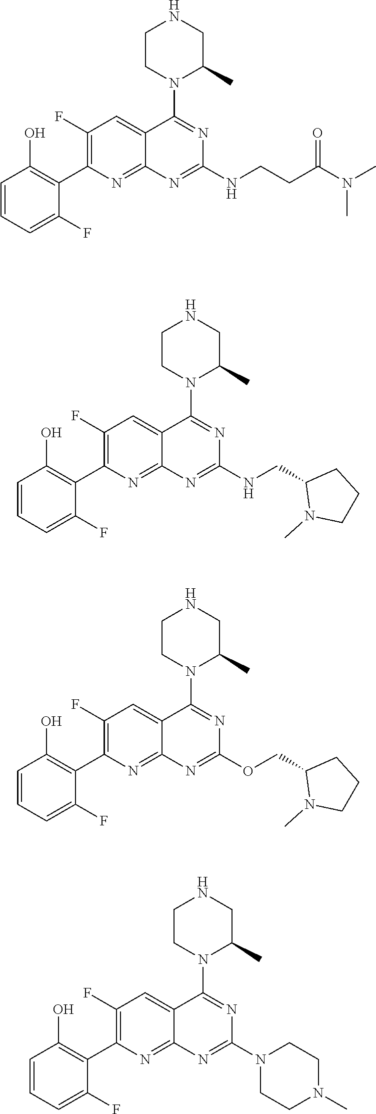 [SMILES: C[C@@H]1CNCCN1c1nc(NCCC(=O)N(C)C)nc2nc(-c3c(O)cccc3F)c(F)cc12 ; C[C@@H]1CNCCN1c1nc(NC[C@@H]2CCCN2C)nc2nc(-c3c(O)cccc3F)c(F)cc12 ; C[C@@H]1CNCCN1c1nc(OC[C@@H]2CCCN2C)nc2nc(-c3c(O)cccc3F)c(F)cc12 ; C[C@@H]1CNCCN1c1nc(N2CCN(C)CC2)nc2nc(-c3c(O)cccc3F)c(F)cc12]

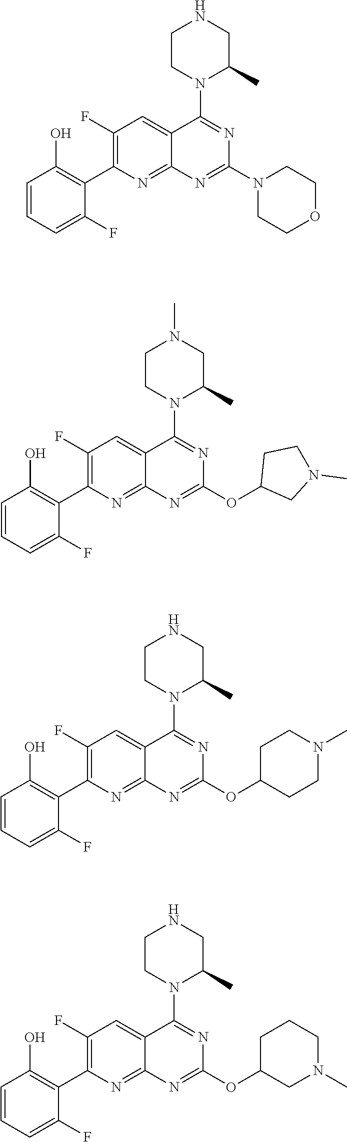 [SMILES: C[C@@H]1CNCCN1c1nc(N2CCOCC2)nc2nc(-c3c(O)cccc3F)c(F)cc12 ; C[C@@H]1CN(C)CCN1c1nc(OC2CCN(C)C2)nc2nc(-c3c(O)cccc3F)c(F)cc12 ; C[C@@H]1CNCCN1c1nc(OC2CCN(C)CC2)nc2nc(-c3c(O)cccc3F)c(F)cc12 ; C[C@@H]1CNCCN1c1nc(OC2CCCN(C)C2)nc2nc(-c3c(O)cccc3F)c(F)cc12]

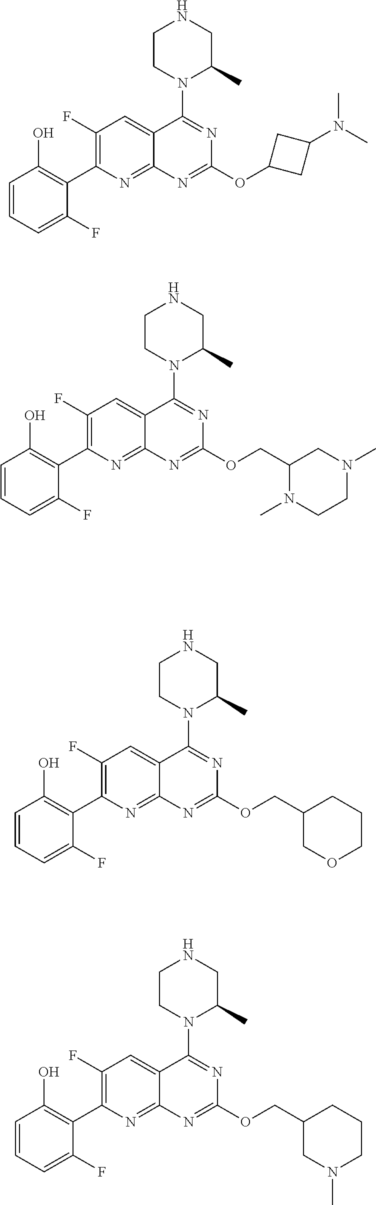 [SMILES: C[C@@H]1CNCCN1c1nc(OC2CC(N(C)C)C2)nc2nc(-c3c(O)cccc3F)c(F)cc12 ; C[C@@H]1CNCCN1c1nc(OCC2CN(C)CCN2C)nc2nc(-c3c(O)cccc3F)c(F)cc12 ; C[C@@H]1CNCCN1c1nc(OCC2CCCOC2)nc2nc(-c3c(O)cccc3F)c(F)cc12 ; C[C@@H]1CNCCN1c1nc(OCC2CCCN(C)C2)nc2nc(-c3c(O)cccc3F)c(F)cc12]

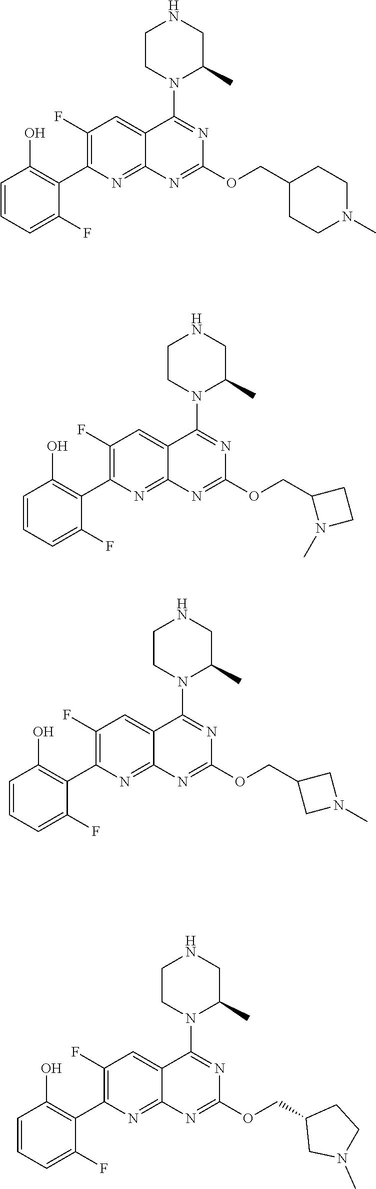 [SMILES: C[C@@H]1CNCCN1c1nc(OCC2CCN(C)CC2)nc2nc(-c3c(O)cccc3F)c(F)cc12 ; C[C@@H]1CNCCN1c1nc(OCC2CCN2C)nc2nc(-c3c(O)cccc3F)c(F)cc12 ; C[C@@H]1CNCCN1c1nc(OCC2CN(C)C2)nc2nc(-c3c(O)cccc3F)c(F)cc12 ; C[C@@H]1CNCCN1c1nc(OC[C@@H]2CCN(C)C2)nc2nc(-c3c(O)cccc3F)c(F)cc12]

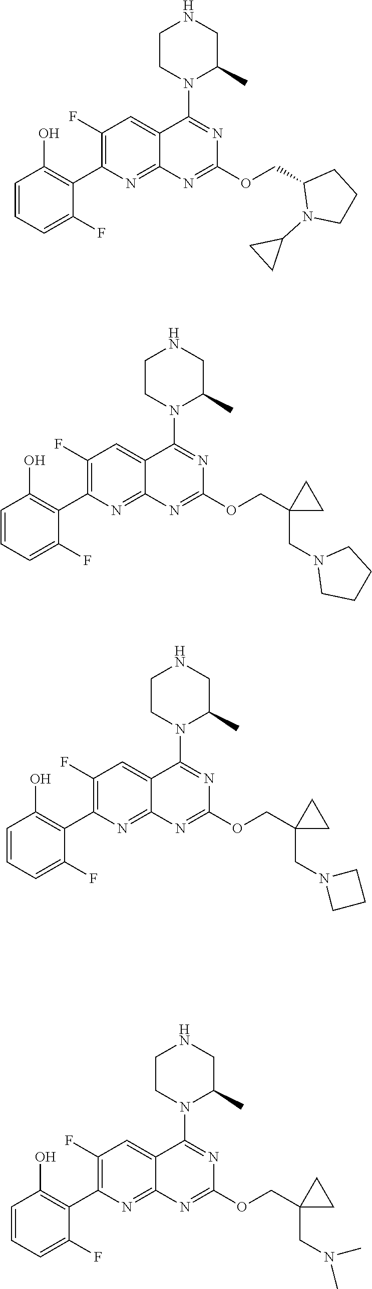 [SMILES: C[C@@H]1CNCCN1c1nc(OC[C@@H]2CCCN2C2CC2)nc2nc(-c3c(O)cccc3F)c(F)cc12 ; C[C@@H]1CNCCN1c1nc(OCC2(CN3CCCC3)CC2)nc2nc(-c3c(O)cccc3F)c(F)cc12 ; C[C@@H]1CNCCN1c1nc(OCC2(CN3CCC3)CC2)nc2nc(-c3c(O)cccc3F)c(F)cc12 ; C[C@@H]1CNCCN1c1nc(OCC2(CN(C)C)CC2)nc2nc(-c3c(O)cccc3F)c(F)cc12]

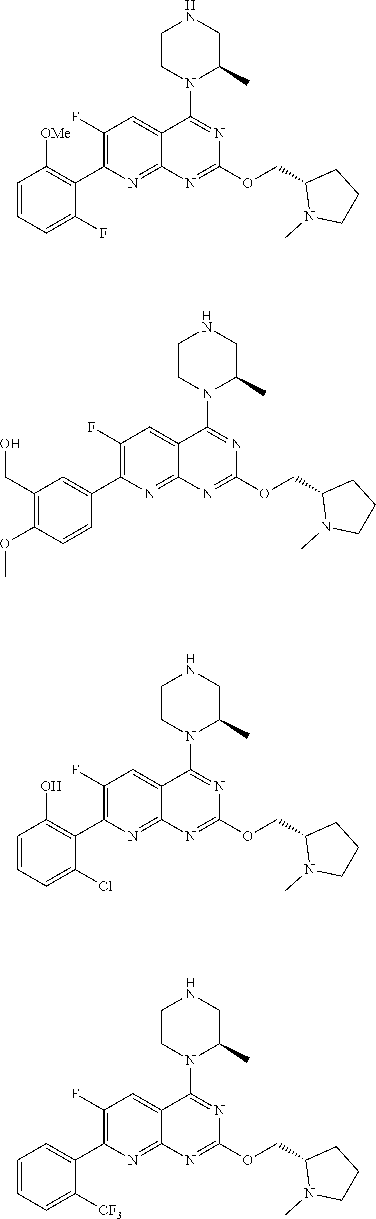 [SMILES: COc1cccc(F)c1-c1nc2nc(OC[C@@H]3CCCN3C)nc(N3CCNC[C@H]3C)c2cc1F ; COc1ccc(-c2nc3nc(OC[C@@H]4CCCN4C)nc(N4CCNC[C@H]4C)c3cc2F)cc1CO ; C[C@@H]1CNCCN1c1nc(OC[C@@H]2CCCN2C)nc2nc(-c3c(O)cccc3Cl)c(F)cc12 ; C[C@@H]1CNCCN1c1nc(OC[C@@H]2CCCN2C)nc2nc(-c3ccccc3C(F)(F)F)c(F)cc12]

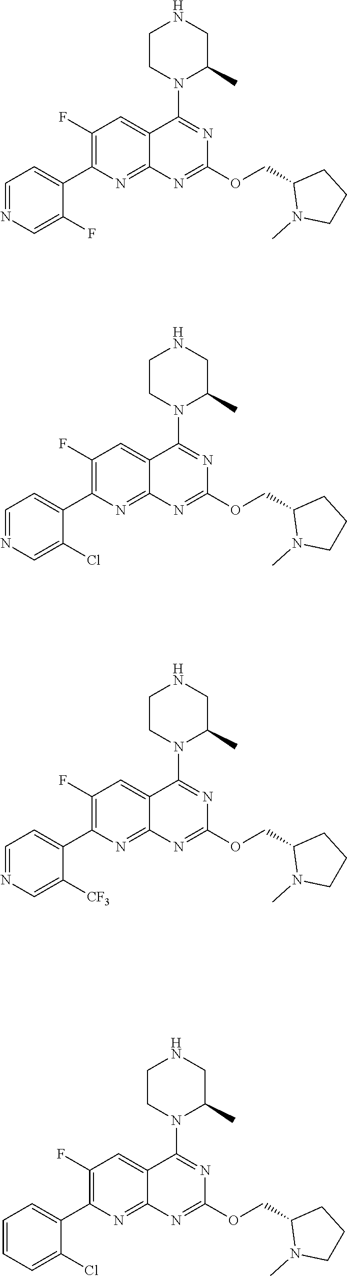 [SMILES: C[C@@H]1CNCCN1c1nc(OC[C@@H]2CCCN2C)nc2nc(-c3ccncc3F)c(F)cc12 ; C[C@@H]1CNCCN1c1nc(OC[C@@H]2CCCN2C)nc2nc(-c3ccncc3Cl)c(F)cc12 ; C[C@@H]1CNCCN1c1nc(OC[C@@H]2CCCN2C)nc2nc(-c3ccncc3C(F)(F)F)c(F)cc12 ; C[C@@H]1CNCCN1c1nc(OC[C@@H]2CCCN2C)nc2nc(-c3ccccc3Cl)c(F)cc12]

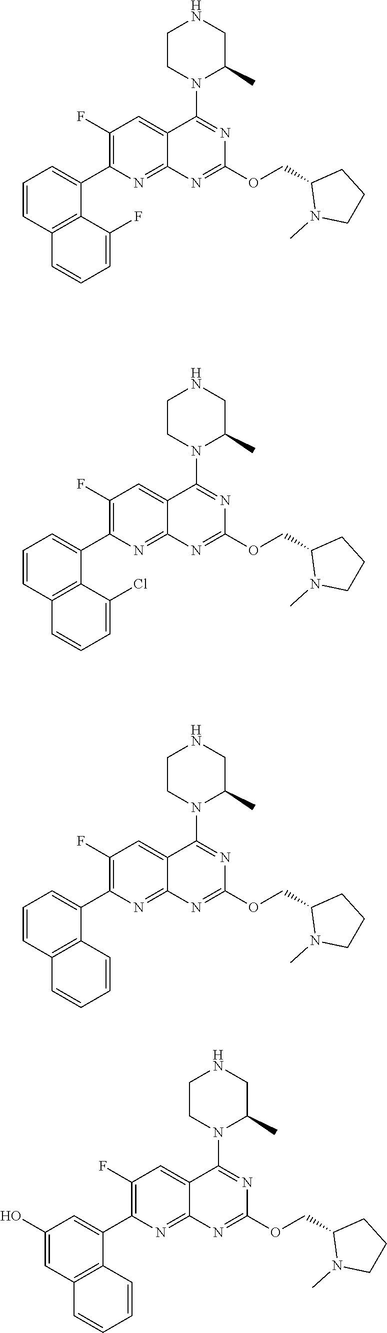 [SMILES: C[C@@H]1CNCCN1c1nc(OC[C@@H]2CCCN2C)nc2nc(-c3cccc4cccc(F)c34)c(F)cc12 ; C[C@@H]1CNCCN1c1nc(OC[C@@H]2CCCN2C)nc2nc(-c3cccc4cccc(Cl)c34)c(F)cc12 ; C[C@@H]1CNCCN1c1nc(OC[C@@H]2CCCN2C)nc2nc(-c3cccc4ccccc34)c(F)cc12 ; C[C@@H]1CNCCN1c1nc(OC[C@@H]2CCCN2C)nc2nc(-c3cc(O)cc4ccccc34)c(F)cc12]

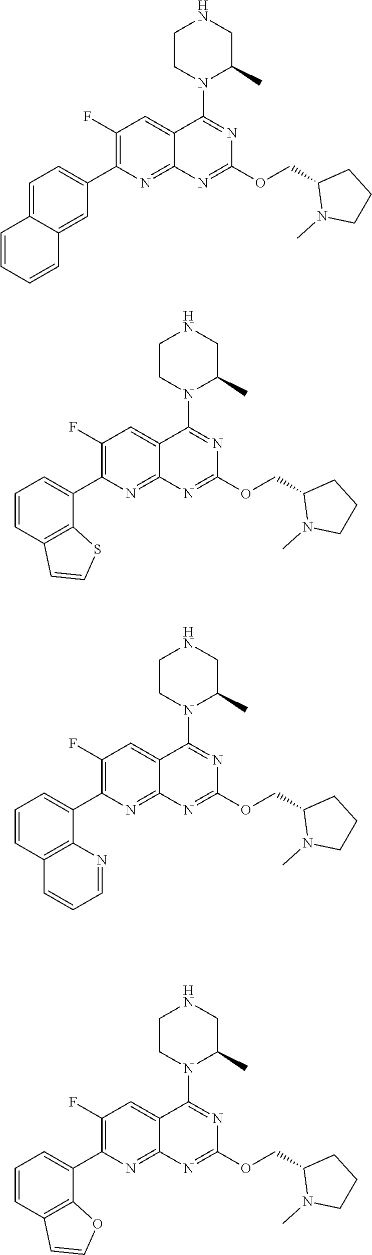 [SMILES: C[C@@H]1CNCCN1c1nc(OC[C@@H]2CCCN2C)nc2nc(-c3ccc4ccccc4c3)c(F)cc12 ; C[C@@H]1CNCCN1c1nc(OC[C@@H]2CCCN2C)nc2nc(-c3cccc4ccsc34)c(F)cc12 ; C[C@@H]1CNCCN1c1nc(OC[C@@H]2CCCN2C)nc2nc(-c3cccc4cccnc34)c(F)cc12 ; C[C@@H]1CNCCN1c1nc(OC[C@@H]2CCCN2C)nc2nc(-c3cccc4ccoc34)c(F)cc12]

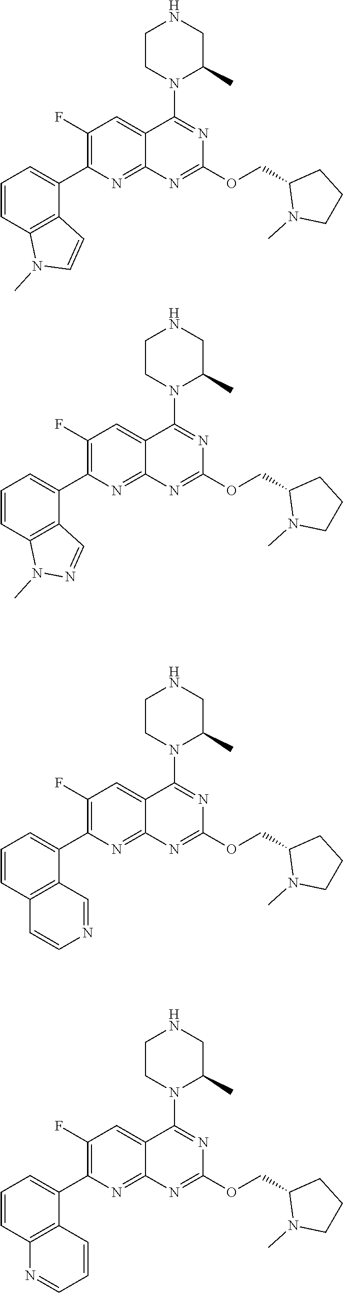 [SMILES: C[C@@H]1CNCCN1c1nc(OC[C@@H]2CCCN2C)nc2nc(-c3cccc4c3ccn4C)c(F)cc12 ; C[C@@H]1CNCCN1c1nc(OC[C@@H]2CCCN2C)nc2nc(-c3cccc4c3cnn4C)c(F)cc12 ; C[C@@H]1CNCCN1c1nc(OC[C@@H]2CCCN2C)nc2nc(-c3cccc4ccncc34)c(F)cc12 ; C[C@@H]1CNCCN1c1nc(OC[C@@H]2CCCN2C)nc2nc(-c3cccc4ncccc34)c(F)cc12]

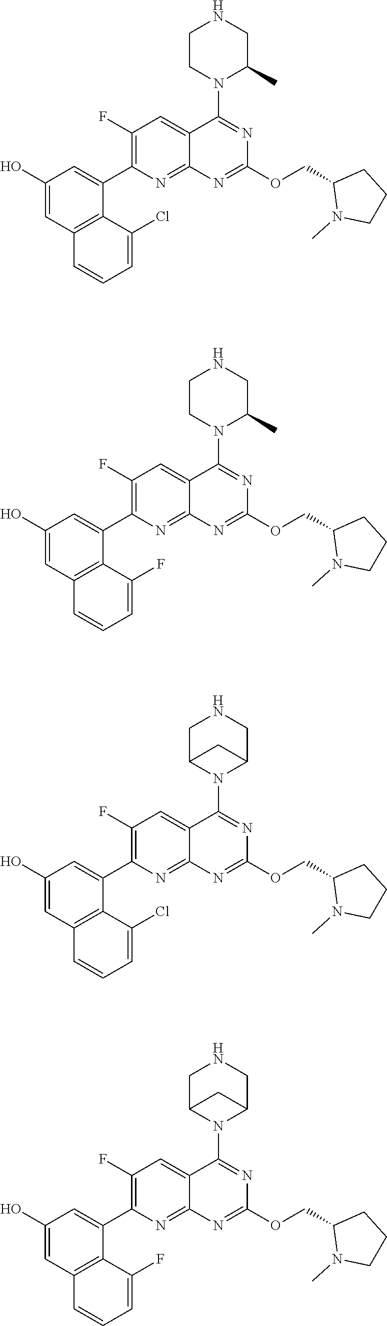 [SMILES: C[C@@H]1CNCCN1c1nc(OC[C@@H]2CCCN2C)nc2nc(-c3cc(O)cc4cccc(Cl)c34)c(F)cc12 ; C[C@@H]1CNCCN1c1nc(OC[C@@H]2CCCN2C)nc2nc(-c3cc(O)cc4cccc(F)c34)c(F)cc12 ; CN1CCC[C@H]1COc1nc(N2C3CNCC2C3)c2cc(F)c(-c3cc(O)cc4cccc(Cl)c34)nc2n1 ; CN1CCC[C@H]1COc1nc(N2C3CNCC2C3)c2cc(F)c(-c3cc(O)cc4cccc(F)c34)nc2n1]

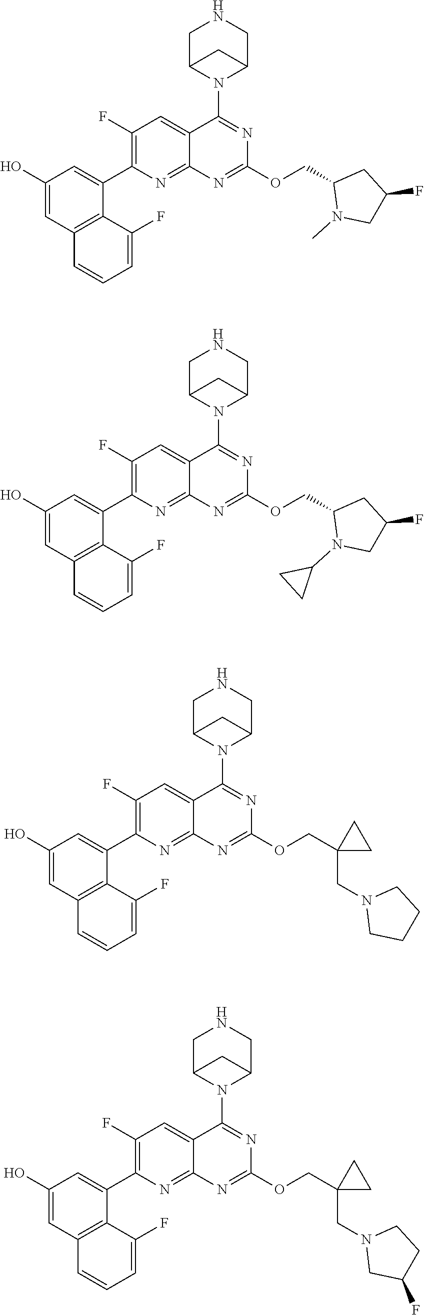 [SMILES: CN1C[C@H](F)C[C@H]1COc1nc(N2C3CNCC2C3)c2cc(F)c(-c3cc(O)cc4cccc(F)c34)nc2n1 ; Oc1cc(-c2nc3nc(OC[C@@H]4C[C@@H](F)CN4C4CC4)nc(N4C5CNCC4C5)c3cc2F)c2c(F)cccc2c1 ; Oc1cc(-c2nc3nc(OCC4(CN5CCCC5)CC4)nc(N4C5CNCC4C5)c3cc2F)c2c(F)cccc2c1 ; Oc1cc(-c2nc3nc(OCC4(CN5CC[C@@H](F)C5)CC4)nc(N4C5CNCC4C5)c3cc2F)c2c(F)cccc2c1]

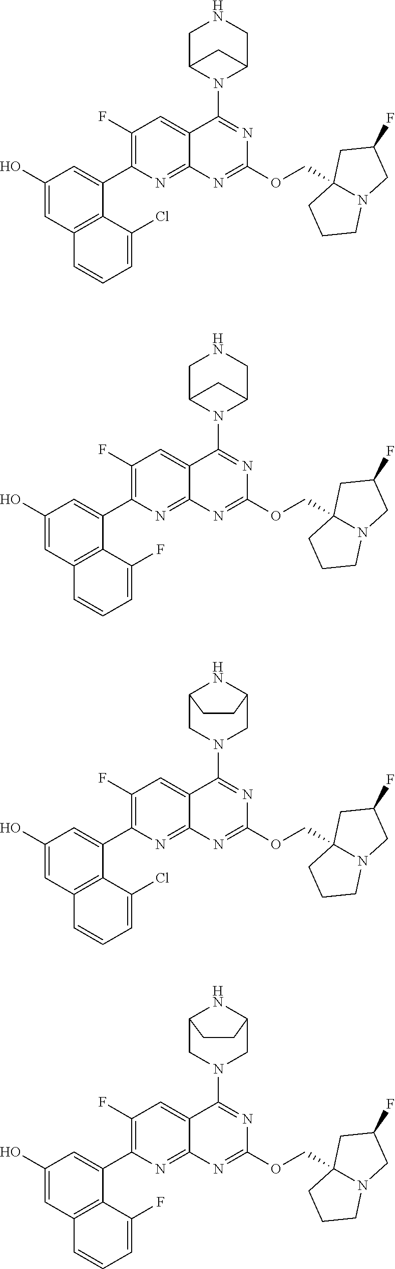 [SMILES: Oc1cc(-c2nc3nc(OC[C@@]45CCCN4C[C@H](F)C5)nc(N4C5CNCC4C5)c3cc2F)c2c(Cl)cccc2c1 ; Oc1cc(-c2nc3nc(OC[C@@]45CCCN4C[C@H](F)C5)nc(N4C5CNCC4C5)c3cc2F)c2c(F)cccc2c1 ; Oc1cc(-c2nc3nc(OC[C@@]45CCCN4C[C@H](F)C5)nc(N4CC5CCC(C4)N5)c3cc2F)c2c(Cl)cccc2c1 ; Oc1cc(-c2nc3nc(OC[C@@]45CCCN4C[C@H](F)C5)nc(N4CC5CCC(C4)N5)c3cc2F)c2c(F)cccc2c1]

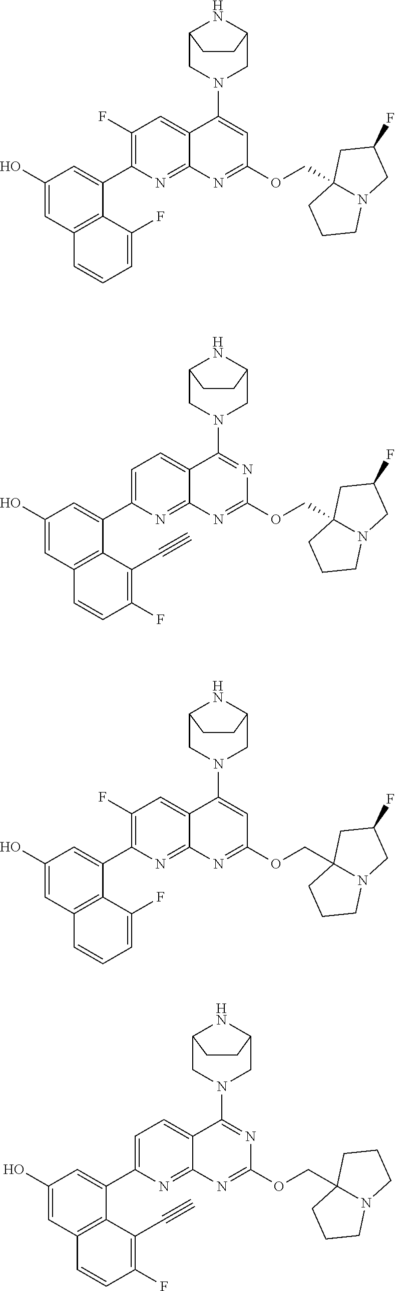 [SMILES: Oc1cc(-c2nc3nc(OC[C@@]45CCCN4C[C@H](F)C5)cc(N4CC5CCC(C4)N5)c3cc2F)c2c(F)cccc2c1 ; C#Cc1c(F)ccc2cc(O)cc(-c3ccc4c(N5CC6CCC(C5)N6)nc(OC[C@@]56CCCN5C[C@H](F)C6)nc4n3)c12 ; Oc1cc(-c2nc3nc(OCC45CCCN4C[C@H](F)C5)cc(N4CC5CCC(C4)N5)c3cc2F)c2c(F)cccc2c1 ; C#Cc1c(F)ccc2cc(O)cc(-c3ccc4c(N5CC6CCC(C5)N6)nc(OCC56CCCN5CCC6)nc4n3)c12]

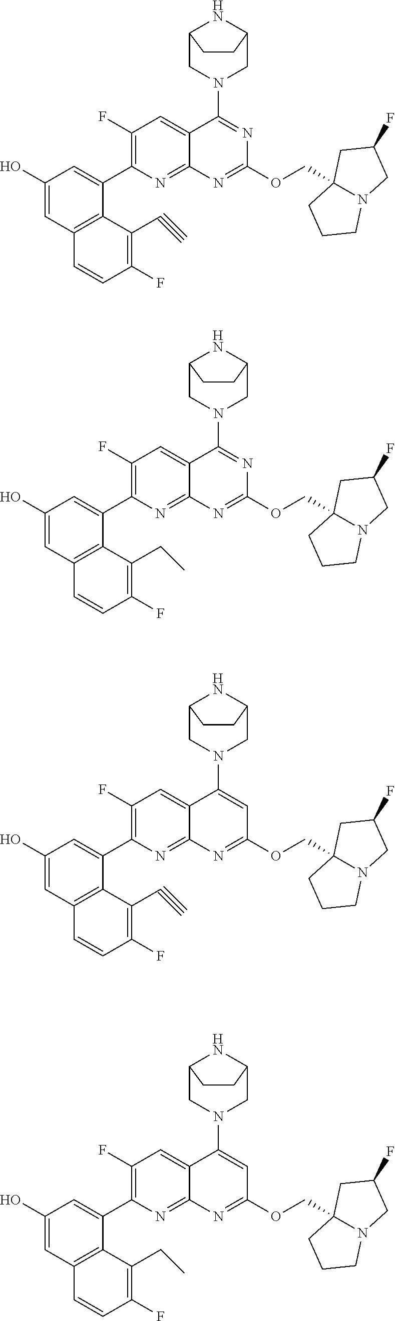 [SMILES: C#Cc1c(F)ccc2cc(O)cc(-c3nc4nc(OC[C@@]56CCCN5C[C@H](F)C6)nc(N5CC6CCC(C5)N6)c4cc3F)c12 ; CCc1c(F)ccc2cc(O)cc(-c3nc4nc(OC[C@@]56CCCN5C[C@H](F)C6)nc(N5CC6CCC(C5)N6)c4cc3F)c12 ; C#Cc1c(F)ccc2cc(O)cc(-c3nc4nc(OC[C@@]56CCCN5C[C@H](F)C6)cc(N5CC6CCC(C5)N6)c4cc3F)c12 ; CCc1c(F)ccc2cc(O)cc(-c3nc4nc(OC[C@@]56CCCN5C[C@H](F)C6)cc(N5CC6CCC(C5)N6)c4cc3F)c12]

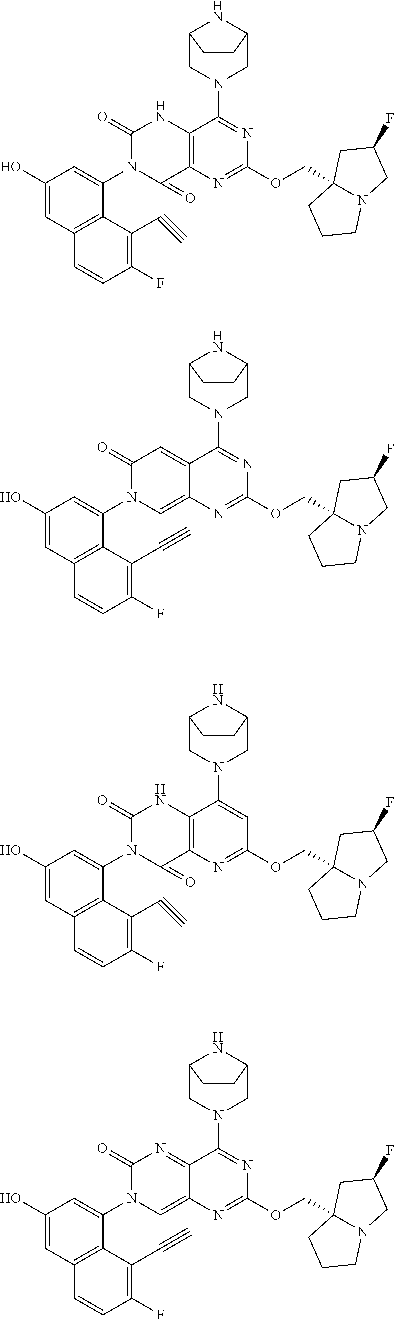 [SMILES: C#Cc1c(F)ccc2cc(O)cc(-n3c(=O)[nH]c4c(N5CC6CCC(C5)N6)nc(OC[C@@]56CCCN5C[C@H](F)C6)nc4c3=O)c12 ; C#Cc1c(F)ccc2cc(O)cc(-n3cc4nc(OC[C@@]56CCCN5C[C@H](F)C6)nc(N5CC6CCC(C5)N6)c4cc3=O)c12 ; C#Cc1c(F)ccc2cc(O)cc(-n3c(=O)[nH]c4c(N5CC6CCC(C5)N6)cc(OC[C@@]56CCCN5C[C@H](F)C6)nc4c3=O)c12 ; C#Cc1c(F)ccc2cc(O)cc(-n3cc4nc(OC[C@@]56CCCN5C[C@H](F)C6)nc(N5CC6CCC(C5)N6)c4nc3=O)c12]

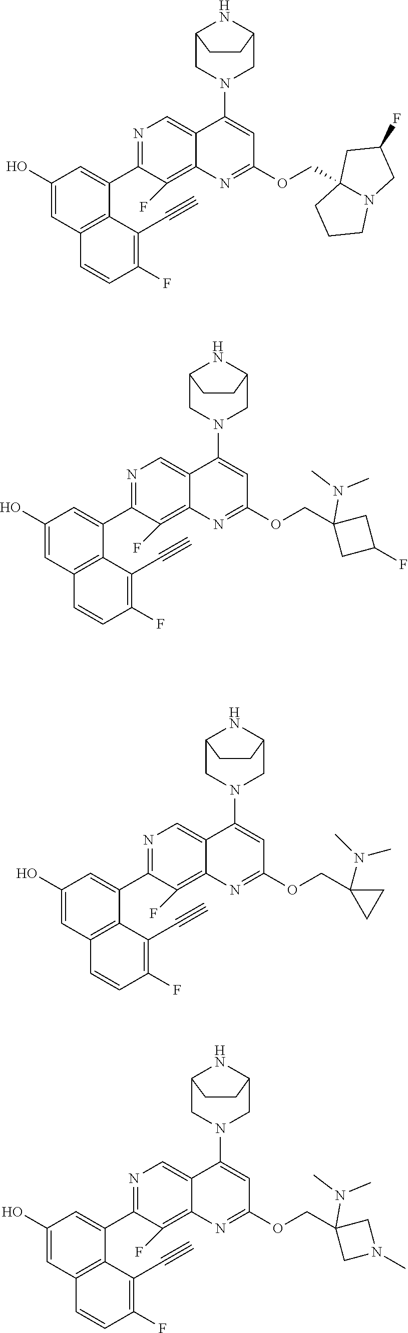 [SMILES: C#Cc1c(F)ccc2cc(O)cc(-c3ncc4c(N5CC6CCC(C5)N6)cc(OC[C@@]56CCCN5C[C@H](F)C6)nc4c3F)c12 ; C#Cc1c(F)ccc2cc(O)cc(-c3ncc4c(N5CC6CCC(C5)N6)cc(OCC5(N(C)C)CC(F)C5)nc4c3F)c12 ; C#Cc1c(F)ccc2cc(O)cc(-c3ncc4c(N5CC6CCC(C5)N6)cc(OCC5(N(C)C)CC5)nc4c3F)c12 ; C#Cc1c(F)ccc2cc(O)cc(-c3ncc4c(N5CC6CCC(C5)N6)cc(OCC5(N(C)C)CN(C)C5)nc4c3F)c12]

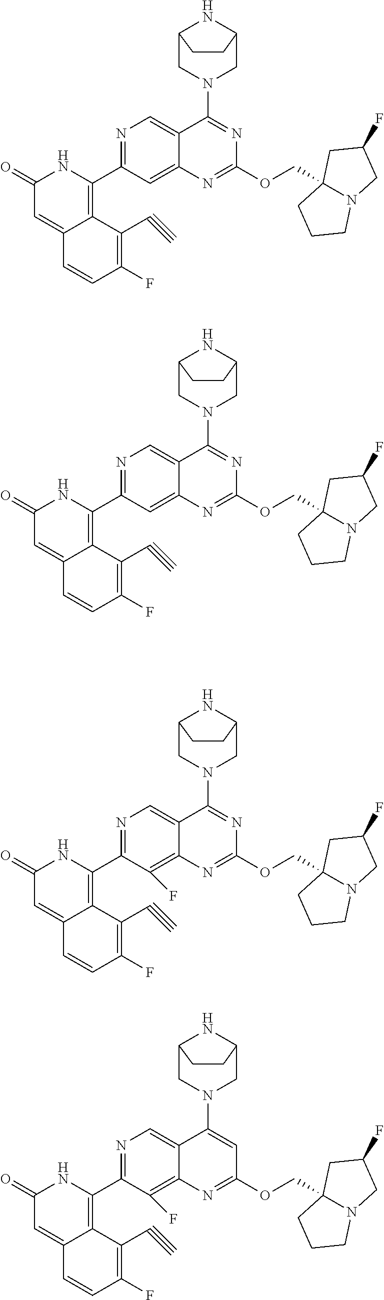 [SMILES: C#Cc1c(F)ccc2cc(=O)[nH]c(-c3cc4nc(OC[C@@]56CCCN5C[C@H](F)C6)nc(N5CC6CCC(C5)N6)c4cn3)c12 ; C#Cc1c(F)ccc2cc(=O)[nH]c(-c3cc4nc(OC[C@@]56CCCN5C[C@H](F)C6)nc(N5CC6CCC(C5)N6)c4cn3)c12 ; C#Cc1c(F)ccc2cc(=O)[nH]c(-c3ncc4c(N5CC6CCC(C5)N6)nc(OC[C@@]56CCCN5C[C@H](F)C6)nc4c3F)c12 ; C#Cc1c(F)ccc2cc(=O)[nH]c(-c3ncc4c(N5CC6CCC(C5)N6)cc(OC[C@@]56CCCN5C[C@H](F)C6)nc4c3F)c12]

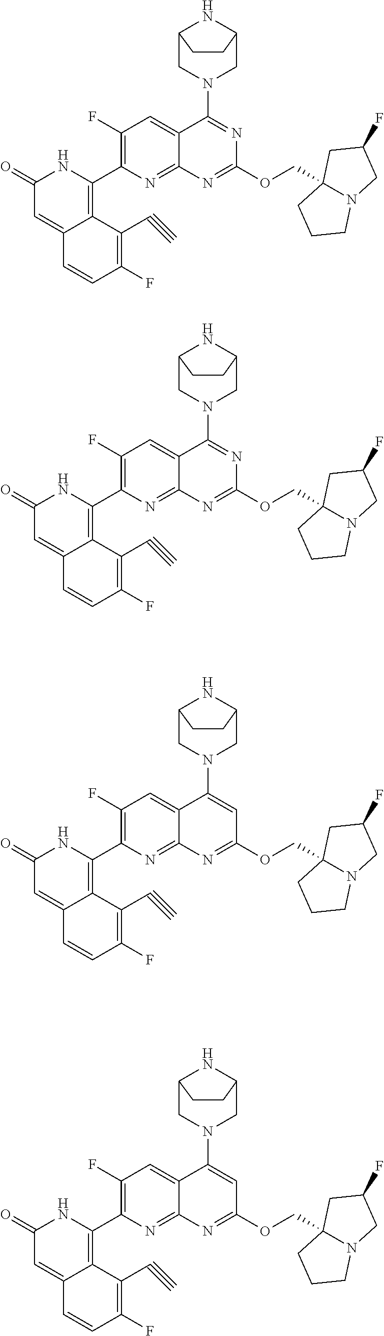 [SMILES: C#Cc1c(F)ccc2cc(=O)[nH]c(-c3nc4nc(OC[C@@]56CCCN5C[C@H](F)C6)nc(N5CC6CCC(C5)N6)c4cc3F)c12 ; C#Cc1c(F)ccc2cc(=O)[nH]c(-c3nc4nc(OC[C@@]56CCCN5C[C@H](F)C6)nc(N5CC6CCC(C5)N6)c4cc3F)c12 ; C#Cc1c(F)ccc2cc(=O)[nH]c(-c3nc4nc(OC[C@@]56CCCN5C[C@H](F)C6)cc(N5CC6CCC(C5)N6)c4cc3F)c12 ; C#Cc1c(F)ccc2cc(=O)[nH]c(-c3nc4nc(OC[C@@]56CCCN5C[C@H](F)C6)cc(N5CC6CCC(C5)N6)c4cc3F)c12]

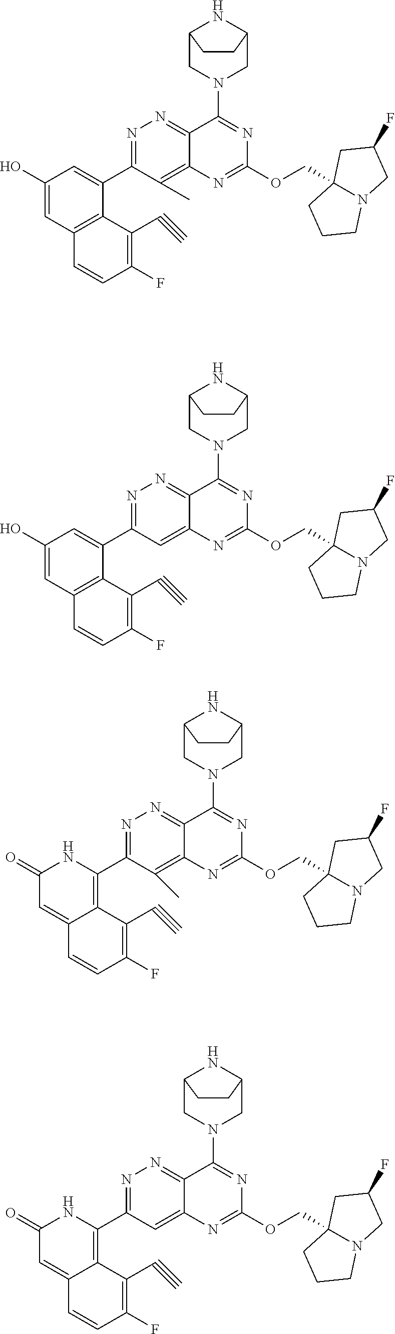 [SMILES: C#Cc1c(F)ccc2cc(O)cc(-c3nnc4c(N5CC6CCC(C5)N6)nc(OC[C@@]56CCCN5C[C@H](F)C6)nc4c3C)c12 ; C#Cc1c(F)ccc2cc(O)cc(-c3cc4nc(OC[C@@]56CCCN5C[C@H](F)C6)nc(N5CC6CCC(C5)N6)c4nn3)c12 ; C#Cc1c(F)ccc2cc(=O)[nH]c(-c3nnc4c(N5CC6CCC(C5)N6)nc(OC[C@@]56CCCN5C[C@H](F)C6)nc4c3C)c12 ; C#Cc1c(F)ccc2cc(=O)[nH]c(-c3cc4nc(OC[C@@]56CCCN5C[C@H](F)C6)nc(N5CC6CCC(C5)N6)c4nn3)c12]

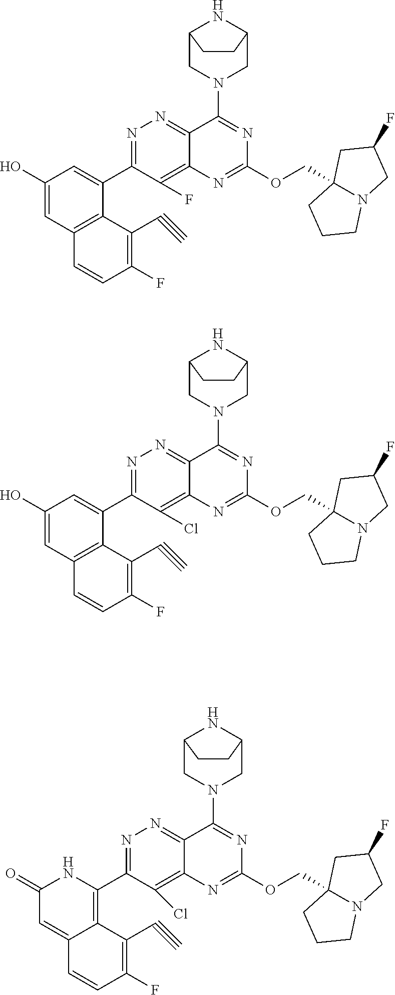 [SMILES: C#Cc1c(F)ccc2cc(O)cc(-c3nnc4c(N5CC6CCC(C5)N6)nc(OC[C@@]56CCCN5C[C@H](F)C6)nc4c3F)c12 ; C#Cc1c(F)ccc2cc(O)cc(-c3nnc4c(N5CC6CCC(C5)N6)nc(OC[C@@]56CCCN5C[C@H](F)C6)nc4c3Cl)c12 ; C#Cc1c(F)ccc2cc(=O)[nH]c(-c3nnc4c(N5CC6CCC(C5)N6)nc(OC[C@@]56CCCN5C[C@H](F)C6)nc4c3Cl)c12]

On the other hand, the present invention also provides a pharmaceutical composition comprising the compound represented by formula (I) or a pharmaceutically acceptable salt thereof and a pharmaceutically acceptable excipient.

In another aspect, the present invention relates to a method for treating a disease related to KRAS G¹²D in a mammal, comprising administering a therapeutically effective amount of a compound represented by formula (I) or a pharmaceutically acceptable compound thereof to a mammal in need of the treatment, preferably a human being salt, or a pharmaceutical composition thereof.

In another aspect, the present invention relates to the use of the compound represented by formula (I) or a pharmaceutically acceptable salt thereof in a medicament for preventing or treating KRAS G12D-related diseases.

In another aspect. the present invention relates to a compound represented by formula (I) or a pharmaceutically acceptable salt thereof, or a pharmaceutical composition thereof for preventing or treating KRAS G¹²D-related diseases.

### Chemical Terms

Unless stated to the contrary, the following terms are used in the specification and claims.

The notation “C_{x-y}” as used herein means a range of carbon atoms, where x and y are both integers, e.g. C₃₋₈ cycloalkyl means a cycloalkyl group having 3-8 carbon atoms, ie a cycloalkyl group having 3, 4, 5, 6, 7 or 8 carbon atoms. It should also be understood that “C₃₋₈” also includes any subrange therein, such as C₃₋₇, C₃₋₆, C₄₋₇, C₄₋₆, C₅₋₆, and the like.

“Alkyl” refers to a straight line containing 1 to 20 carbon atoms, such as 1 to 18 carbon atoms, 1 to 12 carbon atoms, 1 to 8 carbon atoms, 1 to 6 carbon atoms, or 1 to 4 carbon atoms chain or branched hydrocarbyl group. Non-limiting examples of alkyl groups include methyl, ethyl, n-propyl, isopropyl, n-butyl, isobutyl, tert-butyl, sec-butyl, n-pentyl, 1,1-dimethylpropyl 1,2-dimethylpropyl, 2,2-dimethylpropyl, 1-ethylpropyl, 2-methylbutyl, 3-methylbutyl, n-hexyl, 1-ethyl -2-methylpropyl, 1,1,2-trimethylpropyl, 1,1-dimethylbutyl, 1,2-dimethylbutyl, 2,2-dimethylbutyl, 1,3-dimethylbutyl and 2-ethylbutyl. The alkyl group may be substituted or unsubstituted.

“Alkenyl” refers to a straight or branched chain hydrocarbyl group containing at least one carbon-carbon double bond and usually 2 to 20 carbon atoms, such as 2 to 8 carbon atoms, 2 to 6 carbon atoms, or 2 to 4 carbon atoms group. Non-limiting examples of alkenyl groups include vinyl, 1-propenyl, 2-propenyl, 1-butenyl, 2-butenyl, 3-butenyl, 2-methyl-2-propenyl, 1-butenyl, 4-pentadienyl and 1,4-butadienyl. The alkenyl group may be substituted or unsubstituted.

“Alkynyl” refers to a straight or branched chain hydrocarbon group containing at least one carbon-carbon triple bond and usually 2 to 20 carbon atoms, eg, 2 to 8 carbon atoms, 2 to 6 carbon atoms, or 2 to 4 carbon atoms group. Non-limiting examples of alkynyl groups include ethynyl, 1-propynyl, 2-propynyl, 1-butynyl, 2-butynyl, and 3-butynyl. The alkynyl group may be substituted or unsubstituted.

“Cycloalkyl” refers to a saturated cyclic hydrocarbyl substituent containing 3 to 14 carbon ring atoms. Cycloalkyl groups may be monocarbocyclic rings, usually containing 3 to 7 carbon ring atoms. Non-limiting examples of monocyclic cycloalkyl groups include cyclopropyl, cyclobutyl, cyclopentyl, cyclohexyl, and cycloheptyl. Cycloalkyl groups may optionally be bi- or tricyclic rings fused together, such as decalinyl, which may be substituted or unsubstituted.

“Heterocyclyl”, “heterocycloalkyl” and “heterocycle” refer to a stable 3-18-membered monovalent non-aromatic ring, including 2-12 carbon atoms, 1-6 atoms selected from nitrogen, oxygen and sulfur heteroatoms. Unless otherwise specified, a heterocyclyl group may be a monocyclic, bicyclic, tricyclic or tetracyclic ring system, which may contain fused, spiro or bridged ring systems, with nitrogen, carbon or sulfur selectivity on the heterocyclyl group is oxidized, the nitrogen atom can be selectively quaternized, and the heterocyclic group can be partially or fully saturated. A heterocyclyl group can be attached to the rest of the molecule by a single bond through a carbon atom or a heteroatom in the ring. A heterocyclyl group containing a fused ring may contain one or more aromatic or heteroaromatic rings, so long as the attachment to the remainder of the molecule is an atom on a non-aromatic ring. For the purposes of this application, the heterocyclyl group is preferably a stable 4-11 membered monovalent non-aromatic monocyclic or bicyclic ring containing 1-3 heteroatoms selected from nitrogen, oxygen and sulfur, more preferably a stable 4-8 membered monovalent non-aromatic monocyclic ring containing 1-3 heteroatoms selected from nitrogen, oxygen and sulfur. Non-limiting examples of heterocyclyl groups include azepanyl, azetidinyl, decahydroisoquinolyl, dihydrofuranyl, indoline, dioxolane, 1,1-Dioxo-thiomorpholinyl, imidazolidinyl, imidazolinyl, isothiazolidinyl, isoxazolidinyl, morpholinyl, octahydroindolyl, octahydroisoindolyl, oxazinyl, piper oxazinyl, piperidinyl, 4-piperidinyl, pyranyl, pyrazolidine, pyrrolidinyl, quinazinyl, quinuclidinyl, tetrahydrofuranyl, tetrahydropyranyl and the like.

“Spiroheterocyclyl” refers to a 5- to 20-membered polycyclic heterocyclic group with one atom (called a spiro atom) shared between the monocyclic rings, wherein one or more ring atoms are selected from nitrogen, oxygen or S(O)m (where m is an integer from 0 to 2) and the remaining ring atoms are carbon. These may contain one or more double bonds, but none of the rings have a fully conjugated electron system preferably 6 to 14 membered, more preferably 7 to 10 membered. Spirocycloalkyl groups are classified into mono-spiroheterocyclyl, bis-spiro-heterocyclyl or poly-spiro-heterocyclyl according to the number of spiro atoms shared between rings, preferably mono-spirocycloalkyl and bis-spirocycloalkyl. More preferably, it is a 4-membered/4-membered, 4-membered/5-membered, 4-membered/6-membered, 5-membered/5-membered or 5-membered/6-membered monospirocyclic group. Non-limiting examples of spiroheterocyclyl include:

“Fused heterocyclic group” refers to a 5- to 20-membered polycyclic heterocyclic group in which each ring in the system shares an adjacent pair of atoms with other rings in the system, and one or more rings may contain one or more bicyclic bonds, but none of the rings have a fully conjugated pi electron system, where one or more ring atoms are selected from nitrogen, oxygen, or a heteroatom of S(O)ₘ (where m is an integer from 0 to 2), and the remaining ring atoms are carbon. Preferably it is 6 to 14 yuan, more preferably 7 to 10 yuan. According to the number of formed rings, it can be divided into bicyclic, tricyclic, tetracyclic or polycyclic fused heterocycloalkyl, preferably bicyclic or tricyclic, more preferably 5-membered/5-membered or 5-membered/6-membered bicyclic fused heterocyclic group. Non-limiting examples of fused heterocyclyl groups include:

“Aryl” refers to an aromatic monocyclic or fused polycyclic group containing 6 to 14 carbon atoms, preferably 6 to 10 membered, such as phenyl and naphthyl, more preferably phenyl. The aryl ring can be fused to a heteroaryl, heterocyclyl or cycloalkyl ring, wherein the ring attached to the parent structure is an aryl ring.

“Heteroaryl” or “heteroaryl” refers to a 5-16 membered ring system comprising 1-15 carbon atoms, preferably 1-10 carbon atoms, 1-4 selected from nitrogen, oxygen and sulfur heteroatoms, at least one aromatic ring. Unless otherwise specified, a heteroaryl group may be a monocyclic, bicyclic, tricyclic or tetracyclic ring system, which may contain fused or bridged ring systems, so long as the point of attachment to the rest of the molecule is an aromatic ring atom, the Nitrogen, carbon and sulfur atoms can be selectively oxidized, and nitrogen atoms can be selectively quaternized. For the purposes of the present invention, the heteroaryl group is preferably a stable 4-11 membered monoaromatic ring containing 1-3 heteroatoms selected from nitrogen, oxygen and sulfur, more preferably a stable 5-8 membered monoaromatic ring, which contains 1-3 heteroatoms selected from nitrogen, oxygen and sulfur. Non-limiting examples of heteroaryl groups include acridinyl, azepinyl, benzimidazolyl, benzindolyl, benzodioxinyl, benzodioxinyl, benzofuranonyl, benzoyl furanyl, benzonaphthofuryl, benzopyranone, benzopyranyl, benzopyrazolyl, benzothiadiazolyl, benzothiazolyl, benzotriazolyl, furanyl, Imidazolyl, indazolyl, indolyl, oxazolyl, purinyl, pyrazinyl, pyrazolyl, pyridazinyl, pyridyl, pyrimidinyl, pyrrolyl, quinazolinyl, quinolinyl, quinine base, tetrazolyl, thiadiazolyl, thiazolyl, thienyl, triazinyl, triazolyl, etc. In the present application, the heteroaryl group is preferably a 5-8 membered heteroaryl group containing 1-3 heteroatoms selected from nitrogen, oxygen and sulfur, more preferably pyridyl, pyrimidinyl, thiazolyl. The heteroaryl group may be substituted or unsubstituted.

“Halogen” refers to fluorine, chlorine, bromine or iodine.

“Hydroxy” refers to —OH, “amino” refers to —NH₂, “amido” refers to —NHCO—, “cyano” refers to —CN, “nitro” refers to —CN, “isocyano” refers to —NC, “tri “Fluoromethyl” refers to —CF₃.

The term “heteroatom” or “hetero” as used herein, alone or as part of other components, refers to atoms other than carbon and hydrogen, the heteroatoms being independently selected from oxygen, nitrogen, sulfur, phosphorus, silicon, selenium, and tin, Without being limited to these atoms, in embodiments where two or more heteroatoms are present, the two or more heteroatoms may be the same as each other, or some or all of the two or more heteroatoms may be different.

The term “fused” or “fused ring” as used herein, alone or in combination, refers to a cyclic structure in which two or more rings share one or more bonds.

The term “spiro” or “spirocycle” as used herein, alone or in combination, refers to a cyclic structure in which two or more rings share one or more atoms.

“Optional” or “optionally” means that the subsequently described event or circumstance may but need not occur, and that the description includes instances where the event or circumstance occurs or does not occur. For example, “heterocyclyl optionally substituted with alkyl “Group” means that an alkyl group may, but need not, be present, and the description includes the case where the heterocyclic group is substituted with the alkyl group and the case where the heterocyclic group is not substituted with the alkyl group.

“Substituted” means that one or more atoms in a group, preferably 5, more preferably 1 to 3 atoms, are independently of each other substituted with the corresponding number of substituents. It goes without saying that the substituents are in their possible chemical positions, and the person skilled in the art can determine (either experimentally or theoretically) possible or impossible substitutions without undue effort. For example, carbon atoms with free amine or hydroxyl groups bound to carbon atoms with unsaturated (e.g., olefinic) bonds may be unstable. The substituents include, but are not limited to, hydroxyl, amine, halogen, cyano, C₁₋₆ alkyl, C₁₋₆ alkoxy, C₂₋₆ alkenyl, C₂₋₆ alkynyl, C₃₋₈ cycloalkyl, etc.

“Pharmaceutical composition” refers to a composition comprising one or more of the compounds described herein, or a pharmaceutically acceptable salt or prodrug thereof, and other components such as pharmaceutically acceptable carriers and excipients. The purpose of the pharmaceutical composition is to facilitate the administration to the organism, facilitate the absorption of the active ingredient and then exert the biological activity.

“Isomers” refer to compounds that have the same molecular formula but differ in the nature or order in which their atoms are bonded or in the spatial arrangement of their atoms. “. Stereoisomers include optical isomers, geometric isomers and conformational isomers. The compounds of the present invention may exist in the form of optical isomers. These optical isomers are of the “R” or “S” configuration, depending on the configuration of the substituents around the chiral carbon atom. Optical isomers include enantiomers and diastereomers, and methods of making and separating optical isomers are known in the art.

The compounds of the present invention may also exist as geometric isomers. The present invention contemplates various geometric isomers and mixtures thereof resulting from the distribution of substituents around carbon-carbon double bonds, carbon-nitrogen double bonds, cycloalkyl or heterocyclic groups. Substituents around a carbon-carbon double bond or carbon-nitrogen bond are designated as the Z or E configuration, and substituents around a cycloalkyl or heterocycle are designated as the cis or trans configuration.

The compounds of the present invention may also exhibit tautomerism, such as keto-enol tautomerism.

It is to be understood that the present invention includes any tautomeric or stereoisomeric form and mixtures thereof, and is not limited to any one tautomeric or stereoisomeric form used in the naming of the compounds or chemical formulae.

“Isotopes” are all isotopes of atoms occurring in the compounds of the present invention. Isotopes include those atoms with the same atomic number but different mass numbers. Examples of isotopes suitable for incorporation into the compounds of the present invention are hydrogen, carbon, nitrogen, oxygen, phosphorus, fluorine, and chlorine, such as, but not limited to, ²H, ³H, ¹³C, ¹⁴C, ¹⁵N, ¹⁸O, ³¹P, ³²P, ³⁵S, ¹⁸F, respectively, and ³⁶Cl. Isotopically labeled compounds of the present invention can generally be prepared by conventional techniques known to those skilled in the art or by methods analogous to those described in the accompanying Examples, using the appropriate isotopically labeled reagents in place of non-isotopically labeled reagents. Such compounds have various potential uses, e.g., as standards and reagents in the determination of biological activity. In the case of stable isotopes, such compounds have the potential to advantageously alter biological, pharmacological or pharmacokinetic properties.

“Prodrug” means that a compound of the present invention can be administered in the form of a prodrug. Prodrugs refer to derivatives that are converted into biologically active compounds of the present invention under physiological conditions in vivo, e.g., by oxidation, reduction, hydrolysis. etc., each with or without enzymes. Examples of prodrugs are compounds in which the amine group in the compounds of the present invention is acylated, alkylated or phosphorylated, such as eicosanoylamino, propylamineamido, pivaloyloxymethylamine, or wherein the hydroxyl group is acylated, alkylated, phosphorylated or converted to a boronate salt such as acetoxy, palmitoyloxy, pivaloyloxy, succinyloxy, fumaryloxy, alanyloxy Oxygen groups, or in which the carboxyl group is esterified or amidated, or in which the sulfhydryl group forms disulfide bridges with carrier molecules such as peptides that selectively deliver drugs to the target and/or to the cytosol of cells, these compounds may be used by the present invention The compounds are prepared according to known methods.

“Pharmaceutically acceptable salt” or “pharmaceutically acceptable” means prepared from pharmaceutically acceptable bases or acids, including inorganic bases or acids and organic bases or acids. Where the compounds of the present invention contain one or more acidic or basic groups, the present invention also includes their corresponding pharmaceutically acceptable salts. Thus, the compounds of the invention which contain acidic groups can exist in salt form and can be used according to the invention, for example as alkali metal salts, alkaline earth metal salts or as ammonium salts. More precise examples of such salts include sodium, potassium, calcium, magnesium salts or with amines or organic amines such as primary, secondary, tertiary, cyclic, etc., such as ammonia, isopropylamine, trimethylamine, dimethine Particularly preferred organic bases such as ethylamine, triethylamine, tripropylamine, ethanolamine, diethanolamine, ethanolamine, dicyclohexylamine, ethylenediamine, purine, piperazine, piperidine, choline and caffeine are isopropylamine, Salts of ethylamine, ethanolamine, trimethylamine, dicyclohexylamine, choline and caffeine. The compounds of the invention which contain basic groups can exist in salt form and can be used according to the invention in the form of their additions to inorganic or organic acids. Examples of suitable acids include hydrochloric acid, hydrobromic acid, phosphoric acid, sulfuric acid, phosphoric acid, methanesulfonic acid, p-toluenesulfonic acid, naphthalenedisulfonic acid, oxalic acid, acetic acid, tartaric acid, lactic acid, salicylic acid, benzoic acid, formic acid, propylene acid, pivalic acid, malonic acid, succinic acid, pimelic acid, fumaric acid, maleic acid, malic acid, sulfamic acid, phenylpropionic acid, gluconic acid, ascorbic acid, isonicotinic acid, citric acid, adipic acid and other acids known to those skilled in the art. If the compounds of the present invention contain both acidic and basic groups in the molecule, the present invention includes, in addition to the salt forms mentioned, inner or betaine salts. The respective salts are obtained by conventional methods known to those skilled in the art, for example by contacting these with organic or inorganic acids or bases in solvents or dispersants or by anion exchange or cation exchange with other salts.

Accordingly, when referring to “a compound”, “a compound of the present invention” or “a compound of the present invention” in this application, it includes all such compound forms, such as prodrugs, stable isotope derivatives, pharmaceutically acceptable salts, Isomers, mesomers, racemates, enantiomers, diastereomers and mixtures thereof.

As used herein, the term “tumor” includes benign tumors and malignant tumors (e.g., cancer).

As used herein, the term “cancer” includes various malignancies in which Bruton's tyrosine kinases are involved, including but not limited to non-small cell lung cancer, esophageal cancer, melanoma, rhabdomyosarcoma, cell carcinoma, multiple myeloma, Breast ovarian cancer, uterine lining cancer, cervical cancer, stomach cancer, node cancer, bladder cancer, pancreatic cancer, lung cancer, breast cancer, prostate cancer and liver cancer (e.g. hepatocellular carcinoma), more particularly liver cancer, stomach cancer and bladder cancer.

As used herein, the terms “effective amount,” “therapeutically effective amount,” or “pharmaceutically effective amount” refer to at least one agent or compound sufficient to alleviate, to some extent, one or more symptoms of the disease or disorder being treated upon administration amount. The result can be a reduction and/or remission of signs, symptoms or causes or any other desired change in a biological system. For example, an “effective amount” for treatment is that amount of a composition comprising a compound disclosed herein required to provide clinically significant relief of a condition. An effective amount appropriate in any individual case can be determined using techniques such as dose escalation assays.

As used herein, the term “polymorph” or “polymorph (phenomenon)” means that the compounds of the present invention have multiple crystal lattice forms. Some compounds of the present invention may have more than one crystal form. The present invention covers All polymorphs or mixtures thereof.

Intermediate compounds of the compounds of the present invention and polymorphs thereof are also within the scope of the present invention.

Crystallization often results in solvates of the compounds of the present invention, and the term “solvate” as used herein refers to a complex consisting of one or more molecules of the compound of the present invention in combination with one or more molecules of a solvent.

The solvent may be water, in which case the solvate is a hydrate. In addition, an organic solvent may be used. Accordingly, the compounds of the present invention may exist as hydrates, including monohydrates, dihydrates, hemihydrates, trihydrates, tetrahydrates, and the like, as well as the corresponding solvated forms. The compounds of the present invention may be true solvates, but in other cases, the compounds of the present invention may only accidentally retain water or a mixture of water and some other solvent. The compounds of the present invention may be reacted in a solvent or in a solvent Precipitation or crystallization. Solvates of the compounds of the present invention are also included within the scope of the present invention.

The term “acceptable” as used herein in relation to a formulation, composition or ingredient means no persistent detrimental effect on the general health of the subject being treated.

As used herein, the term “pharmaceutically acceptable” refers to a substance (eg, a carrier or diluent) that does not affect the biological activity or properties of the compounds of the present invention, and is relatively non-toxic, i.e., the substance can be administered to an individual without causing adverse biological effects React or interact in an undesirable manner with any component contained in the composition.

“Pharmaceutically acceptable carrier” includes, but is not limited to, adjuvants, carriers, excipients, auxiliaries, deodorants, diluents, preservatives, Dyes/colorants, flavor enhancers, surfactants and wetting agents, dispersing agents, suspending agents, stabilizers, isotonic agents, solvents, or emulsifiers.

The terms “subject”, “patient”, “subject” or “individual” as used herein refer to an individual suffering from a disease, disorder or condition, etc., including mammals and non-mammals, examples of mammals include, but are not limited to, the class of mammals Any member of: humans, non-human primates (e.g. chimpanzees and other apes and monkeys); domestic animals such as cattle, horses, sheep, goats, pigs; domestic animals such as rabbits, dogs and cats; laboratory animals, including rodents such as rats, mice and guinea pigs. Examples of non-human mammals include, but are not limited to, birds, fish, and the like. In one embodiment of the related methods and compositions provided herein, the mammal is a human.

The term “treatment” as used herein refers to the treatment of related disease conditions in mammals, particularly humans, including
(i) preventing mammals, particularly mammals who have been previously exposed to a disease or disorder but have not been diagnosed with the disease or disorder, from developing a corresponding disease or disorder;
(ii) inhibiting the disease or disorder, i.e. controlling its development;
(iii) alleviating the disease or condition, i.e. causing regression of the disease or condition;
(iv) Relief of symptoms caused by a disease or disorder.

As used herein, the terms “disease” and “disorder” can be used interchangeably or have different meanings, because some specific diseases or conditions have no known causative agent (so the cause of the disease is not clear), so they cannot be considered as A disease can only be seen as an unwanted condition or syndrome which has more or less specific symptoms that have been confirmed by clinical researchers.

The terms “administering,” and the like, as used herein, refer to methods that enable the delivery of a compound or composition to the desired site of biological action. These include, but are not limited to, oral routes, duodenal routes, parenteral injection (including intravenous, subcutaneous, intraperitoneal, intramuscular, intraarterial injection or infusion), topical administration, and rectal administration. In preferred embodiments, the compounds and compositions discussed herein are administered orally.

### EXAMPLES

The present invention also provides methods for preparing the compounds. The preparation of the compounds of the general formula (I) of the present invention can be accomplished by the following exemplary methods and examples. but these methods and examples should not be construed as limiting the scope of the present invention in any way. The compounds of the present invention can also be synthesized using synthetic techniques known to those skilled in the art, or a combination of methods known in the art and methods described in the present invention. The products obtained in each step are obtained by separation techniques known in the art, including but not limited to extraction, filtration, distillation, crystallization, chromatographic separation, and the like. The starting materials and chemical reagents required for the synthesis can be routinely synthesized or purchased according to the literature (reaxys).

The compound described in the general formula (I) of the present invention or its isomer, solvate or its precursor, or their pharmaceutically acceptable salt is as follows:

### Method A

1 [symbol] The starting material A1 and the precursor H-U-Y-P undergo an aromatic nucleophilic substitution reaction under the action of a base to generate A2;
2 [symbol] A3 and the precursor R₁₉ undergo aromatic nucleophilic substitution reaction under the action of alkali to form A3;
3 [symbol] A3 is coupled to obtain intermediate A4;
4 [symbol] A4 removes the protecting group to obtain I;

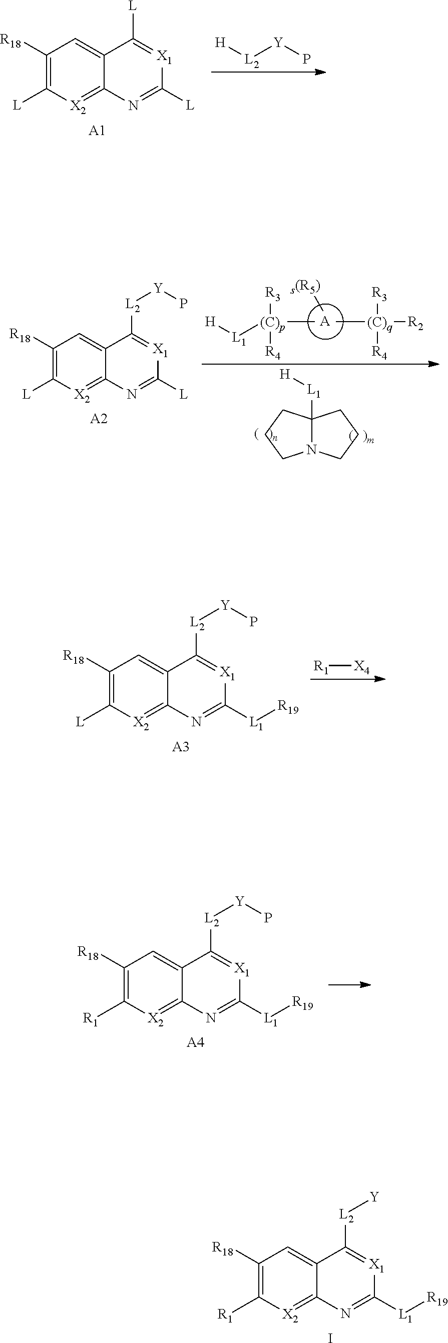

### Method B

1 [symbol] Starting material B1 and intermediate B2 are obtained by coupling;
2 [symbol] B2 and POCl₃ or POBr₃ obtain intermediate B3;
3 [symbol] B3 and the precursor H-U-Y-P undergo aromatic nucleophilic substitution reaction under the action of alkali to form B4;
4 [symbol] The aromatic nucleophilic substitution reaction between B4 and the precursor R₁₉ under the action of alkali generates B5;
5 [symbol] B5 removes the protecting group to obtain I;

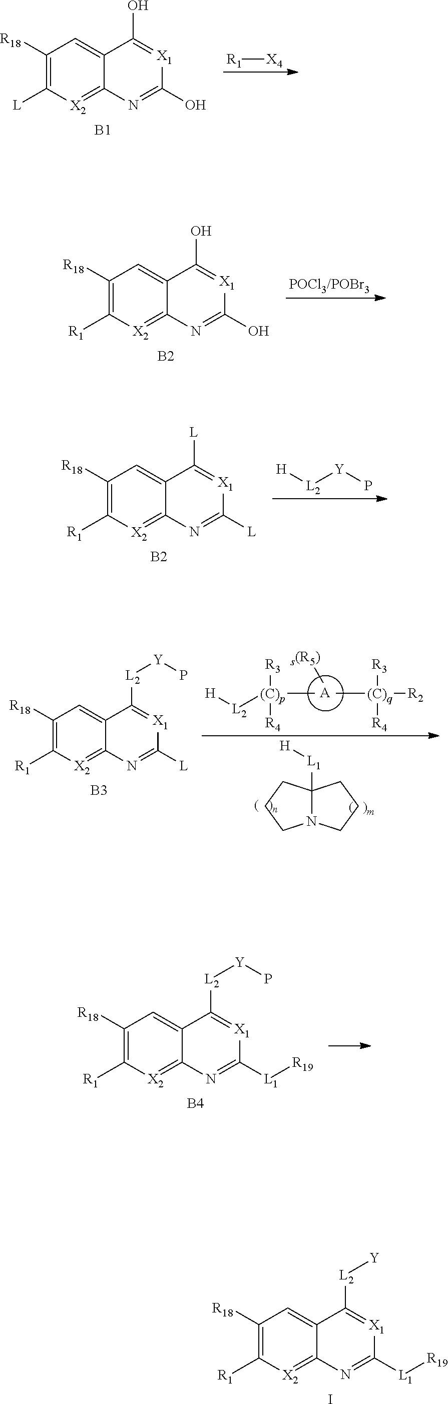

Unless otherwise stated, temperatures are in degrees Celsius. Reagents were purchased from commercial suppliers such as Chemblocks Inc, Astatech Inc, or Maclean, and these reagents were used without further purification unless otherwise stated.

Unless otherwise stated, the following reactions were performed at room temperature, in anhydrous solvent, under positive pressure of nitrogen or gas, or using a drying tube; glassware drying and/or heat drying.

Unless otherwise stated, column chromatography used 200-300 mesh silica gel from Qingdao Ocean Chemical Factory; preparative thin layer chromatography used thin layer chromatography silica gel prefabricated plate (HSGF254) produced by Yantai Chemical Industry Research Institute; MS was measured with Themno LCD Fleet type (ESI) liquid chromatography-mass spectrometer.

Nuclear magnetic data (¹H NMR) use BrukerAvance-400 MHz or Varian Oxford-400 Hz nuclear magnetic instrument, the solvents used for nuclear magnetic data are CDCl₃, CD3OD, D₂O, DMS-d₆, etc., based on tetramethylsilane (0.000 ppm) or residual solvent As the benchmark (CDCl₃: 7.26 ppm: CD₃OD: 3.31 ppm; D₂O: 4.79 ppm; d6-DMSO: 2.50 ppm) When the peak shape diversity is indicated, the following abbreviations indicate different peak shapes: s (single peak), d (double peak)), t (triplet), q (quartet), m (multiplet), br (broad), dd (double doublet), dt (double triplet). If a coupling constant is given, it is in Hertz (Hz).

### Preparation of Intermediates

### (2-(((5-fluoro-4-(4,4,5,5-tetramethyl-1,3,2-dioxaborolan-2-yl)naphthalen-2-yl)oxy)methoxy)ethyl)trimethylsilane

According to the documents Journal of the American Chemical Society, 1976, vol.98. #11, p.3237-3242 and KR102121583:

To a solution of Br₂ (5.2 g, 32.5 mmol) in AcOH (15 mL) was added a solution of 5-fluoronaphthalen-1-amine (2.4 g, 15 mmol) in AcOH (10 mL), the reaction was stirred at 70° C. for 1 h. The reaction mixture was cooled at room temperature and filtered. The filter cake was washed with AcOH (15 mL), then added to 20% aqueous of NaOH (30 mL). The mixture was stirred for 20 min and filtered. The isolated solid was washed with water (20 mL) and dried under vacuum to provide 2,4-dibromo-5-fluoronaphthalen-1-amine (4.45 g. 93%) as a gray solid. LC/MS(ESI): m/z=320[M+H]⁺.

To a solution of 2,4-dibromo-5-fluoronaphthalen-1-amine (3.84 g, 12 mmol) in AcOH (65mL) was added propionic acid (11 mL) at 0° C. Then, 1.2 g of sodium nitrite was added and stirred for 30 minutes. The reaction solution was poured into ice water at 0° C., and the resulting solid was filtered and further added to the filtrate and stirred to produce a yellow precipitate. The resulting precipitate was filtered and dried to obtain 5-bromo-6-fluoronaphtho[1,2-d][1,2,3]oxadiazole (1.35 g, 42%). LC/MS(ESD): m/z=268[M+H]⁺.

To a solution of 5-bromo-6-fluoronaphtho[1,2-d][1,2,3]oxadiazole (1.34 g, 5 mmol) in EtOH (25mL) was added sodium borohydride (0.57 g) under nitrogen atmosphere and stirred for 12 h. An aqueous hydrochloric acid solution containing 28 mL of hydrochloric acid was added dropwise to the reactor and stirred for 1 h. At the end of the reaction, neutralized by adding 10% aqueous sodium hydroxide solution. After neutralization was completed, the mixture was extracted with dichloromethane and water, and the organic layer is concentrated. The residue was purified by flash column to afford 4-bromo-5-fluoronaphthalen-2-ol as a yellow solid (0.92 g, 76%). LC/MS(ESI): m/z=242[M+H]⁺.

Sodium hydride (0.23 g, 5.67 mmol, 60%) was added protionwise to a solution of 4-bromo-5-fluoronaphthalen-2-ol (0.91 g. 3.78 mmol) in THE (10 mL) with stirring at 0° C. Ater 0.5 h, chlorotriisopropylsilane (5.6 g, 5.67 mmol) was added dropwise at 0° C. The solution was stirred at room temperature overnight, poured into water (10 mL), extracted with EtOAc (2×10 mL). The organic layer was dried over anhydrous sodium sulfate, filtered and concentrated to give (2-(((4-bromo-5-fluoronaphthalen-2-yl)oxy)methoxy)ethyl)trimethylsilane (1.35 g, 96%). LC/MS(ESI): m/z=272[M+H]⁺.

Under nitrogen protection, [1,1′-bis(diphenylphosphino)ferrocene]dichloropalladium dichloromethane complex (240 mg, 0.3 mmol) and potassium acetate (726 mg, 7.38 mmol) were added to A solution of (2-(((4-bromo-5-fluoronaphthalen-2-yloxy)methoxy)ethyl)trimethylsilane (816 mg, 3 mmol) and bispinacolatodiboron (980 mg, 3.9 mmol) in 60 mL of DMF The reaction was stirred at 90° C. for 5 hours. The mixture was then diluted with water (100 ml) and extracted with ethyl acetate (100 ml), the organic phase was dried over anhydrous, filtered and the filtrate was concentrated to give crude product. The crude product was purified by column chromatography to give a white solid (905 mg, 72% yield). LC/MS (ESI): m/z=419.2 [M+H]⁺.

### (2-(((5-chloro-4-(4,4,5,5-tetramethyl-1,3,2-dioxaborolan-2-yl)naphthalen-2-yl)oxy)methoxy)ethyl)trimethylsilane

### Step A

To a solution of Br₂ (5.2 g, 32.5 mmol) in AcOH (15 mL) was added a solution of 5-chloronaphthylamine (2.65 g, 15 mmol) in AcOH (10 mL) and the reaction was stirred at 70° C. for 1 h. The reaction mixture was cooled at room temperature and filtered. The filter cake was washed with 15 mL of AcOH, then 20% aqueous NaOH (30 mL) was added. The mixture was stirred for 20 minutes and filtered. The isolated solid was washed with 20 mL of water and dried under vacuum to provide 2,4-dibromo-5-chloronaphthalen-1-amine (4.83 g, 96% yield) as a grey solid. LC/MS (ESI): m/z=336 [M+H]+.

### Step B

2,4-dibromo-5-chloronaphthalen-1-amine (4.03 g, 12 mmol) was dissolved in 65 mL of AcOH and cooled to 0° C. 11 mL of propionic acid was then added and stirred. Then, 1.2 g of sodium nitrite was added, and the mixture was stirred for 30 minutes and several minutes. The reaction solution was poured into ice water at 0° C., the resulting solid was filtered and further added to the filtrate and stirred to produce a yellow precipitate. The resulting precipitate was filtered and dried to obtain the intermediate 5-bromo-6-chloronaphtho[1,2-d][1,2,3]oxadiazole (1.50 g, 44% yield). LC/MS (ESI): m/z=284 [M+H]+.

### Step C

5-bromo-6-chloronaphtho[1,2-d][1,2,3]oxadiazole (1.42 g. 5 mmol) was dissolved in 25 mL EtOH under nitrogen protection, then 0.57 g of hydroboration was added, sodium, and the reaction was stirred for 12 hours. Then, 28 mL of hydrochloric acid solution was added dropwise and stirred for 1 hour. At the end of the reaction, 10% NaOH aqueous solution was added for neutralization. After completion of neutralization, it was extracted with dichloromethane, and the organic layer was concentrated. Flash column purification gave 4-bromo-5-chloronaphthalen-2-ol (1.02 g, 79%). LC/MS (ESI): m/z=259 [M+H]+.

### Step D

4-bromo-5-chloronaphthalen-2-ol (0.973 g, 3.78 mmol) was dissolved in 10 mL of tetrahydrofuran. Then, stirring at 0° C., 60% NaH (1.35 g, 5.67 mmol) was added in several portions. Then, SEMCl (5.6 g, 5.67 mmol) was added. The resulting solution was stirred overnight at 25° C., and then 10 ml of water was added to quench the reaction. The resulting solution was extracted with 2×10 mL of ethyl acetate, and the organic layers were combined. The mixture was dried over anhydrous sodium sulfate and concentrated in vacuo to give crude (2-(((4-bromo-5-chloronaphthalen-2-yl)oxy)methoxy)ethyl)trimethylsilane (1.39 g. 95%). LC/MS (ESI): m/z=388 [M+H]+.

### Step E

Under nitrogen protection, [1,1′-bis(diphenylphosphino)ferrocene]dichloropalladium dichloromethane complex (240 mg, 0.3 mmol) and potassium acetate (726 mg, 7.38 mmol) were added to (2-(((4-bromo-5-chloronaphthalen-2-yl)oxy)methoxy)ethyl)trimethylsilane (1.16 mg, 3 mmol) and bispinacolatodiboron (980 mg, 3.9 mmol) dissolved in 60 mL of DMF The solution was stirred at 90° C. for 5 hours. The mixture was then diluted with water (100 ml) and extracted with ethyl acetate (100 ml), the organic phase was dried over anhydrous, filtered and the filtrate was concentrated to give crude product. The crude product was purified by column chromatography to give a white solid (992 mg, 76% yield). LC/MS (ESI): m/z=436 [M+H]+.

### EXAMPLE 1

### 2-(2-((2-(dimethylamino)ethyl)amino)-6-fluoro-4-((R)-2-methylpiperazin-1-yl)pyrido[2,3-d]pyrimidin-7-yl)-3-fluorophenol (Compound 1)

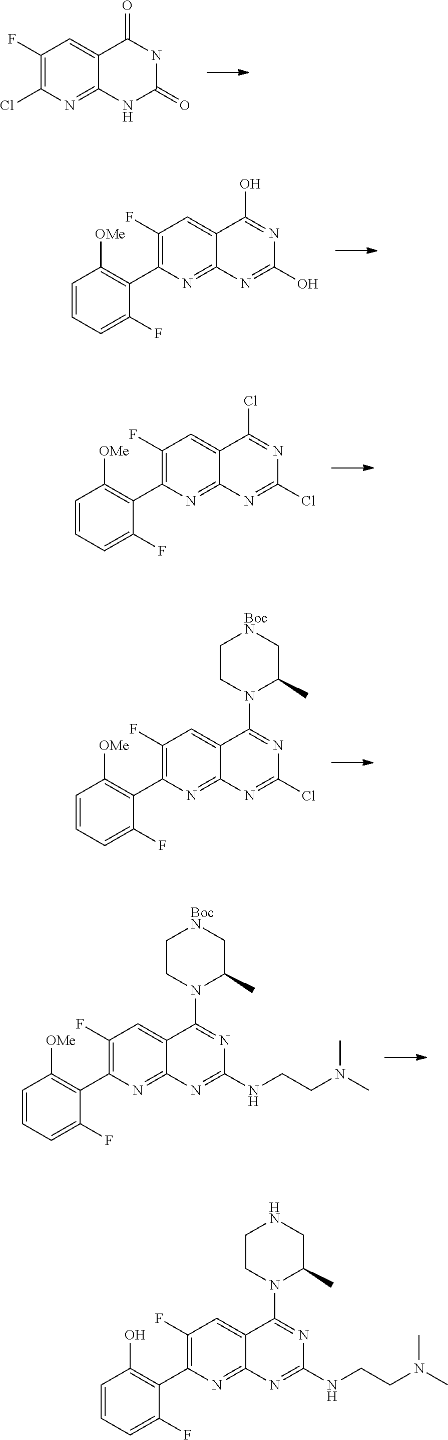 [SMILES: O=c1[nH]c(=O)c2cc(F)c(Cl)nc2[nH]1 ; COc1cccc(F)c1-c1nc2nc(O)nc(O)c2cc1F ; COc1cccc(F)c1-c1nc2nc(Cl)nc(Cl)c2cc1F ; COc1cccc(F)c1-c1nc2nc(Cl)nc(N3CCN(C(=O)OC(C)(C)C)C[C@H]3C)c2cc1F ; COc1cccc(F)c1-c1nc2nc(NCCN(C)C)nc(N3CCN(C(=O)OC(C)(C)C)C[C@H]3C)c2cc1F ; C[C@@H]1CNCCN1c1nc(NCCN(C)C)nc2nc(-c3c(O)cccc3F)c(F)cc12]

### Step 1: 6-fluoro-7-(2-fluoro-6-methoxyphenyl)pyrido[2,3-d]pyrimidine-2,4-diol

7-chloro-6-fluoropyrido[2,3-d]pyrimidine-2,4(1H,3H)-dione (2.17 g. 0.01 mol), 6-methoxy-2-fluorophenylboronic acid (1.7 g, 0.01 mol), tris(dibenzylideneacetone)dipalladium (0.8 g, 0.88 mmol), cesium carbonate, 1,4-dioxane (100 mL) and water (20 mL) were mixed, then heated under reflux To 120° C., the reaction was stirred for 16 hours. The reaction was cooled to room temperature and stirred overnight to give a pale yellow precipitate. The reaction mixture was diluted with water (2 mL) and the solid was collected by filtration. The crude product was slurried with methanol (10 mL) to give 6-fluoro-7-(2-fluoro-6-methoxyphenyl)pyrido [2,3-d]pyrimidine-2,4-diol (2.17 g, 72%) was carried to the next reaction without further purification. LC/MS(ESI): m/z=306[M+H]+.

### Step 2: 2,4-dichloro-6-fluoro-7-(2-fluoro-6-methoxyphenyl)pyrido[2,3-d]pyrimidine

6-fluoro-7-(2-fluoro-6-methoxyphenyl)pyrido[2,3-d]pyrimidine-2,4-diol (1.83 g 6 mmol) was dissolved in POCl₃ (30 mL), a small amount of N,N-xylene amine was added, and the reaction was stirred under reflux for 10 h. Then poured into ice water to quench, filtered to obtain solid product, washed with water, and dried to obtain crude yellow solid 2,4-dichloro-6-fluoro- 7-(2-fluoro-6-methoxyphenyl)pyrido[2,3-d]pyrimidine (1.51 g, 74%) was carried to the next reaction without further purification. LC/MS(ESI): m/z=343[M+H]+.

### Step 3: tert-butyl (3R)-4-(2-chloro-6-fluoro-7-(2-fluoro-6-methoxyphenyl)pyrido[2,3-d]pyrimidin-4-yl)-3-methylpiperazine-1-carboxylate

2,4-dichloro-6-fluoro-7-(2-fluoro-6-methoxyphenyl)pyrido[2,3-d]pyrimidine (1.37 g, 4 mmol), (R)-4-Boc -2-Methylpiperazine (0.88 g, 4.4 mmol), potassium carbonate (0.88 g, 6.4 mmol) catalytic potassium iodide and DMF (80 mL) were mixed, heated to 120° C., and the reaction was stirred for 4 hours. Cooled to room temperature, evaporated under reduced pressure to obtain a yellow solid tert-butyl (3R)-4-(2-chloro-6-fluoro-7-(2-fluoro-6-methoxyphenyl)pyrido[2.3-d]pyrimidin-4-yl)-3-methylpiperazine-1-carboxylate (1.34 g. 68%), LC/MS (ESI): m/z=507 [M+H]+.

### Step 4: tert-butyl (3R)-4-(2-((2-(dimethylamino)ethyl)amino)-6-fluoro-7-(2-fluoro- 6-methoxyphenyl)pyrido[2,3-d]pyrimidin-4-yl)-3-methylpiperazine-1-carboxylate

tert-butyl (3R)-4-(2-chloro-6-fluoro-7-(2-fluoro-6-methoxyphenyl)pyrido[2,3-d]pyrimidin-4-yl)-3-methylpiperazine-1-carboxylate (152 mg, 0.3 mmol), N,N-dimethylethylenediamine (29 mg. 0.33 mmol), potassium carbonate (62 mg, 0.45 mmol) catalytic potassium iodide and DMF (10 mL) were mixed, heated to 120° C. and stirred for 4 hours. Cooled to room temperature, evaporated under reduced pressure, and column chromatography gave a yellow solid tert-butyl (3R)-4-(2-((2-(dimethylamino)ethyl)amino)-6-fluoro-7-(2-fluoro-6-methoxyphenyl)pyrido[2,3-d]pyrimidin-4-yl)-3-methylpiperazine-1-carboxylate (109 mg, 65%). LC/MS (ESI): m/z=558.3 [M+H]+.

### Step 5: 2-(2-((2-(dimethylamino)ethyl)amino)-6-fluoro-4-((R)-2-methylpiperazin-1-yl) pyrido[2,3-d]pyrimidin-7-yl)-3-fluorophenol

tert-butyl(3R)-4-(2-((2-(dimethylamino)ethyl)amino)-6-fluoro-7-(2-fluoro-6-methoxyphenyl)pyrido[2,3-d]pyrimidin-4-yl)-3-methylpiperazine-1-carboxylate (100 mg, 0.18 mmol) was dissolved in 10 mL of DCM, at −78° C. BBr (0.8 mL) was added, then warmed to room temperature and stirred React overnight. The reaction was quenched with water, and extracted with DCM (2*15 mL), the organic layers were combined, dried over anhydrous sodium sulfate, and concentrated to obtain the target product as a yellow solid 2-(2-((2-(dimethylamino)ethyl)amino)-6-fluoro-4-((R)-2- methylpiperazin-1-yl)pyrido[2,3-d]pyrimidin-7-yl)-3-fluorophenol (62 mg, 78%). LC/MS (ESI): m/z=444.2 [M+H]+. 1H NMR(500 MHz, CD3OD) δ8.51 (d,1H), 7.39-7.35 (m,1H), 6.87-6.80 (m,3H), 6.21-6.14 (m,1H), 5.43 (dd,1H), 4.78 (broad s,1H), 4.40-3.96 (m,4H), 3.75-3.45 (m,4H), 3.15-2.95 (m,2H), 2.64 (s,6H), 1.30 (s,3H).

Examples 2-24 refer to the preparation method of compound 1 and its corresponding intermediates

| | | | MS:(M + H)⁺/¹H |
|---|---|---|---|
| Compound | Chemical Name | Structure | NMR |
| 2 | 2-(2-(2- (dimethylamino) ethoxy)-6-fluoro-4- ((R)-2- methylpiperazin-1- yl)pyrido[2,3- d]pyrimidin-7-yl)- 3-fluorophenol | 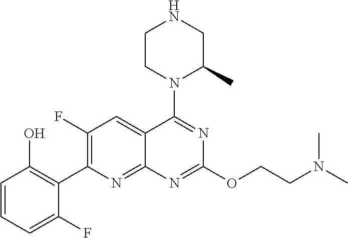 [SMILES: C[C@@H]1CNCCN1c1nc(OCCN(C)C)nc2nc(-c3c(O)cccc3F)c(F)cc12] | LC/MS(ESI): m/z = 445.2[M + H]⁺ |
| 3 | 3-fluoro-2-(6- fluoro-4-((R)-2- methylpiperazin-1- yl)-2-((tetrahydro- 2H-pyran-4- yl)methoxy)pyrido [2,3-d]pyrimidin-7- yl)phenol | 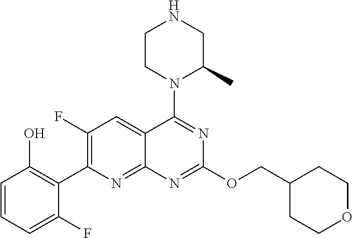 [SMILES: C[C@@H]1CNCCN1c1nc(OCC2CCOCC2)nc2nc(-c3c(O)cccc3F)c(F)cc12] | LC/MS(ESI): m/z = 472.2[M + H]⁺ |
| 4 | 3-fluoro-2-(6- fluoro-4-((R)-2- methylpiperazin-1- yl)-2-(2-(4- methylpiperazin-1- yl)ethoxy)pyrido[2, 3-d]pyrimidin-7- yl)phenol | 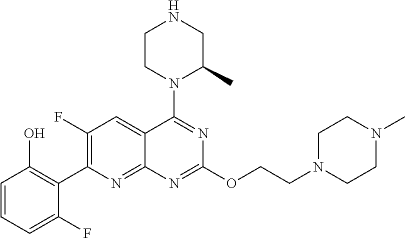 [SMILES: C[C@@H]1CNCCN1c1nc(OCCN2CCN(C)CC2)nc2nc(-c3c(O)cccc3F)c(F)cc12] | LC/MS(ESI): m/z = 500.3[M + H]⁺ |
| 5 | 3-((6-fluoro-7-(2- fluoro-6- hydroxyphenyl)-4- ((R)-2- methylpiperazin-1- yl)pyrido[2,3- d]pyrimidin-2- yl)amino)-N,N- dimethyl- propanamide | 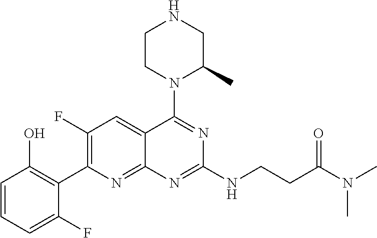 [SMILES: C[C@@H]1CNCCN1c1nc(NCCC(=O)N(C)C)nc2nc(-c3c(O)cccc3F)c(F)cc12] | LC/MS(ESI): m/z = 472.2[M + H]⁺ |
| 6 | 3-fluoro-2-(6- fluoro-4-((R)-2- methylpiperazin-1- yl)-2-((((S)-1- methylpyrrolidin- 2- yl)methyl)amino) pyrido[2,3- d]pyrimidin-7- yl)phenol | 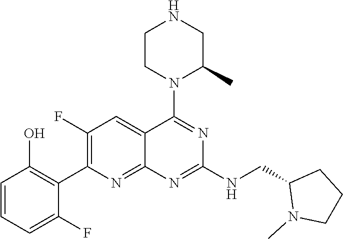 [SMILES: C[C@@H]1CNCCN1c1nc(NC[C@@H]2CCCN2C)nc2nc(-c3c(O)cccc3F)c(F)cc12] | LC/MS(ESI): m/z = 470.2[M + H]⁺ |
| 7 | 3-fluoro-2-(6- fluoro-4-((R)-2- methylpiperazin-1- yl)-2-(((S)-1- methylpyrrolidin- 2- yl)methoxy)pyrido [2,3-d]pyrimidin-7- yl)phenol | 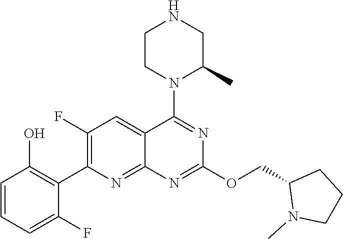 [SMILES: C[C@@H]1CNCCN1c1nc(OC[C@@H]2CCCN2C)nc2nc(-c3c(O)cccc3F)c(F)cc12] | LC/MS(ESI): m/z = 471.2[M + H]⁺ |
| 8 | 3-fluoro-2-(6- fluoro-4-((R)-2- methylpiperazin-1- yl)-2-(4- methylpiperazin-1- yl)pyrido[2,3- d]pyrimidin-7- yl)phenol | 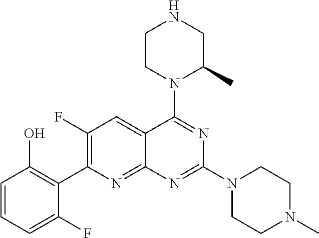 [SMILES: C[C@@H]1CNCCN1c1nc(N2CCN(C)CC2)nc2nc(-c3c(O)cccc3F)c(F)cc12] | LC/MS(ESI): m/z = 456.2[M + H]⁺ |
| 9 | 3-fluoro-2-(6- fluoro-4-((R)-2- methylpiperazin-1- yl)-2- morpholinopyrido [2,3-d]pyrimidin-7- yl)phenol | 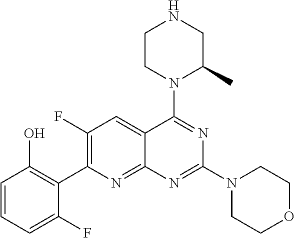 [SMILES: C[C@@H]1CNCCN1c1nc(N2CCOCC2)nc2nc(-c3c(O)cccc3F)c(F)cc12] | LC/MS(ESI): m/z = 443.2[M + H]⁺ |
| 10 | 2-(4-((R)-2,4- dimethylpiperazin- 1-yl)-6-fluoro-2- ((1- methylpyrrolidin- 3- yl)oxy)pyrido[2,3- d]pyrimidin-7-yl)- 3-fluorophenol | 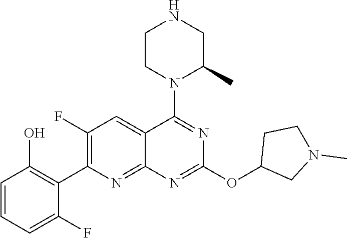 [SMILES: C[C@@H]1CNCCN1c1nc(OC2CCN(C)C2)nc2nc(-c3c(O)cccc3F)c(F)cc12] | LC/MS(ESI): m/z = 457.2[M + H]⁺ |
| 11 | 3-fluoro-2-(6- fluoro-4-((R)-2- methylpiperazin-1- yl)-2-((1- methylpiperidin-4- yl)oxy)pyrido[2,3- d]pyrimidin-7- yl)phenol | 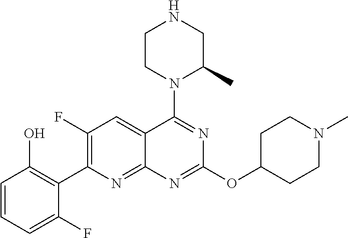 [SMILES: C[C@@H]1CNCCN1c1nc(OC2CCN(C)CC2)nc2nc(-c3c(O)cccc3F)c(F)cc12] | LC/MS(ESI): m/z = 471.2[M + H]⁺ |
| 12 | 3-fluoro-2-(6- fluoro-4-((R)-2- methylpiperazin-1- yl)-2-((1- methylpiperidin-3- yl)oxy)pyrido[2,3- d]pyrimidin-7- yl)phenol | 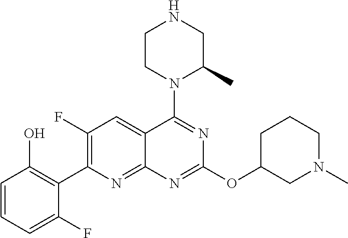 [SMILES: C[C@@H]1CNCCN1c1nc(OC2CCCN(C)C2)nc2nc(-c3c(O)cccc3F)c(F)cc12] | LC/MS(ESI): m/z = 471.2[M + H]⁺ |
| 13 | 2-(2-(3- (dimethylamino) cyclobutoxy)-6- fluoro-4-((R)-2- methylpiperazin-1- yl)pyrido[2,3- d]pyrimidin-7-yl)- 3-fluorophenol | 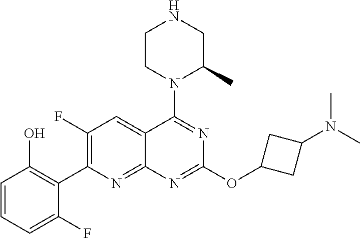 [SMILES: C[C@@H]1CNCCN1c1nc(OC2CC(N(C)C)C2)nc2nc(-c3c(O)cccc3F)c(F)cc12] | LC/MS(ESI): m/z = 471.2[M + H]⁺ |
| 14 | 2-(2-((1,4- dimethylpiperazin- 2-yl)methoxy)-6- fluoro-4-((R)-2- methylpiperazin-1- yl)pyrido[2,3- d]pyrimidin-7-yl)- 3-fluorophenol | 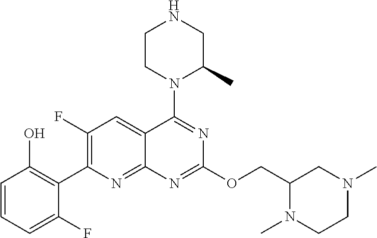 [SMILES: C[C@@H]1CNCCN1c1nc(OCC2CN(C)CCN2C)nc2nc(-c3c(O)cccc3F)c(F)cc12] | LC/MS(ESI): m/z = 500.3[M + H]⁺ |
| 15 | 3-fluoro-2-(6- fluoro-4-((R)-2- methylpiperazin-1- yl)-2-((tetrahydro- 2H-pyran-3- yl)methoxy)pyrido [2,3-d]pyrimidin-7- yl)phenol | 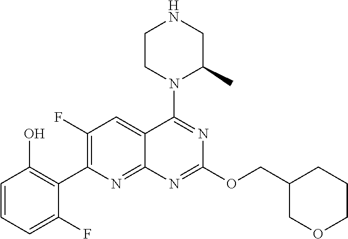 [SMILES: C[C@@H]1CNCCN1c1nc(OCC2CCCOC2)nc2nc(-c3c(O)cccc3F)c(F)cc12] | LC/MS(ESI): m/z = 472.2[M + H]⁺ |
| 16 | 3-fluoro-2-(6- fluoro-4-((R)-2- methylpiperazin-1- yl)-2-((1- methylpiperidin-3- yl)methoxy)pyrido [2,3-d]pyrimidin-7- yl)phenol | 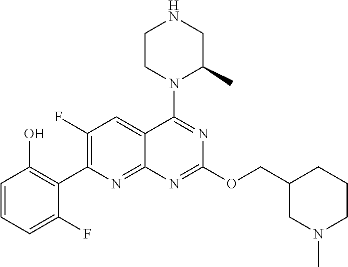 [SMILES: C[C@@H]1CNCCN1c1nc(OCC2CCCN(C)C2)nc2nc(-c3c(O)cccc3F)c(F)cc12] | LC/MS(ESI): m/z = 485.2[M + H]⁺ |
| 17 | 3-fluoro-2-(6- fluoro-4-((R)-2- methylpiperazin-1- yl)-2-((1- methylpiperidin-4- yl)methoxy)pyrido [2,3-d]pyrimidin-7- yl)phenol | 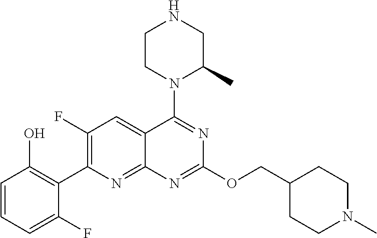 [SMILES: C[C@@H]1CNCCN1c1nc(OCC2CCN(C)CC2)nc2nc(-c3c(O)cccc3F)c(F)cc12] | LC/MS(ESI): m/z = 485.2[M + H]⁺ |
| 18 | 3-fluoro-2-(6- fluoro-2-((1- methylazetidin-2- yl)methoxy)-4- ((R)-2- methylpiperazin-1- yl)pyrido[2,3- d]pyrimidin-7- yl)phenol | 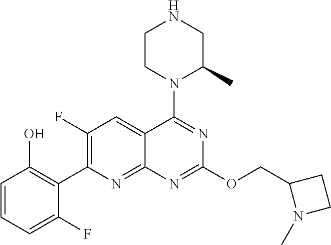 [SMILES: C[C@@H]1CNCCN1c1nc(OCC2CCN2C)nc2nc(-c3c(O)cccc3F)c(F)cc12] | LC/MS(ESI): m/z = 457.2[M + H]⁺ |
| 19 | 3-fluoro-2-(6- fluoro-2-((1- methylazetidin-3- yl)methoxy)-4- ((R)-2- methylpiperazin-1- yl)pyrido[2,3- d]pyrimidin-7- yl)phenol | 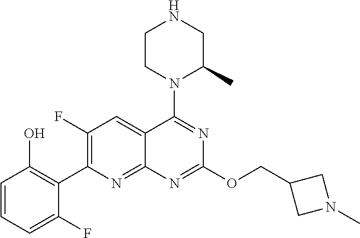 [SMILES: C[C@@H]1CNCCN1c1nc(OCC2CN(C)C2)nc2nc(-c3c(O)cccc3F)c(F)cc12] | LC/MS(ESI): m/z = 457.2[M + H]⁺ |
| 20 | 3-fluoro-2-(6- fluoro-4-((R)-2- methylpiperazin-1- yl)-2-(((R)-1- methylpyrrolidin- 3- yl)methoxy)pyrido [2,3-d]pyrimidin-7- yl)phenol | 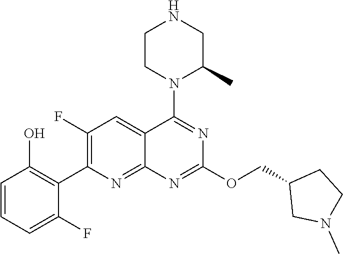 [SMILES: C[C@@H]1CNCCN1c1nc(OC[C@@H]2CCN(C)C2)nc2nc(-c3c(O)cccc3F)c(F)cc12] | LC/MS(ESI): m/z = 471.2[M + H]⁺ |
| 21 | 2-(2-(((S)-1- cyclopropyl- pyrrolidin- 2-yl)methoxy)- 6-fluoro-4-((R)-2- methylpiperazin-1- yl)pyrido[2,3- d]pyrimidin-7-yl)- 3-fluorophenol | 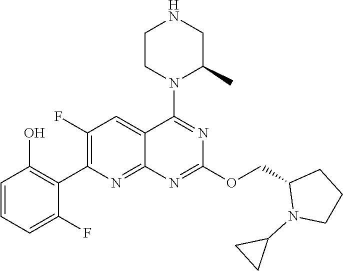 [SMILES: C[C@@H]1CNCCN1c1nc(OC[C@@H]2CCCN2C2CC2)nc2nc(-c3c(O)cccc3F)c(F)cc12] | LC/MS(ESI): m/z = 497.2[M + H]⁺ |
| 22 | 3-fluoro-2-(6- fluoro-4-((R)-2- methylpiperazin-1- yl)-2-((1- (pyrrolidin-1- ylmethyl)cyclopro- pyl)methoxy)pyrido [2,3-d]pyrimidin- 7-yl)phenol | 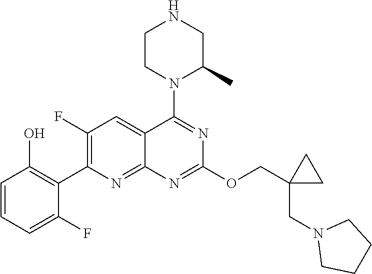 [SMILES: C[C@@H]1CNCCN1c1nc(OCC2(CN3CCCC3)CC2)nc2nc(-c3c(O)cccc3F)c(F)cc12] | LC/MS(ESI): m/z = 511.2[M + H]⁺ |
| 23 | 2-(2-((1-(azetidin- 1- ylmethyl)cyclopro- pyl)methoxy)-6- fluoro-4-((R)-2- methylpiperazin-1- yl)pyrido[2,3- d]pyrimidin-7-yl)- 3-fluorophenol | 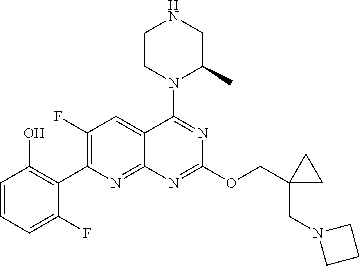 [SMILES: C[C@@H]1CNCCN1c1nc(OCC2(CN3CCC3)CC2)nc2nc(-c3c(O)cccc3F)c(F)cc12] | LC/MS(ESI): m/z = 497.2[M + H]⁺ |
| 24 | 2-(2-((1- ((dimethylamino) methyl)cyclopropyl) methoxy)-6-fluoro- 4-((R)-2- methylpiperazin-1- yl)pyrido[2,3- d]pyrimidin-7-yl)- 3-fluorophenol | 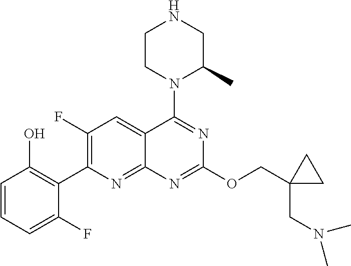 [SMILES: C[C@@H]1CNCCN1c1nc(OCC2(CN(C)C)CC2)nc2nc(-c3c(O)cccc3F)c(F)cc12] | LC/MS(ESI): m/z = 485.2[M + H]⁺ |

### Example 25 ((3R)-4-(6-fluoro-7-(2-fluoro-6-methoxyphenyl)-2-(((S)-1-methylpyrrolidin-2-yl)methoxy)pyrido[2,3-d]pyrimidin-4-yl)-3-methylpiperazin-1-yl)prop-2-en-1-one (Compound 25)

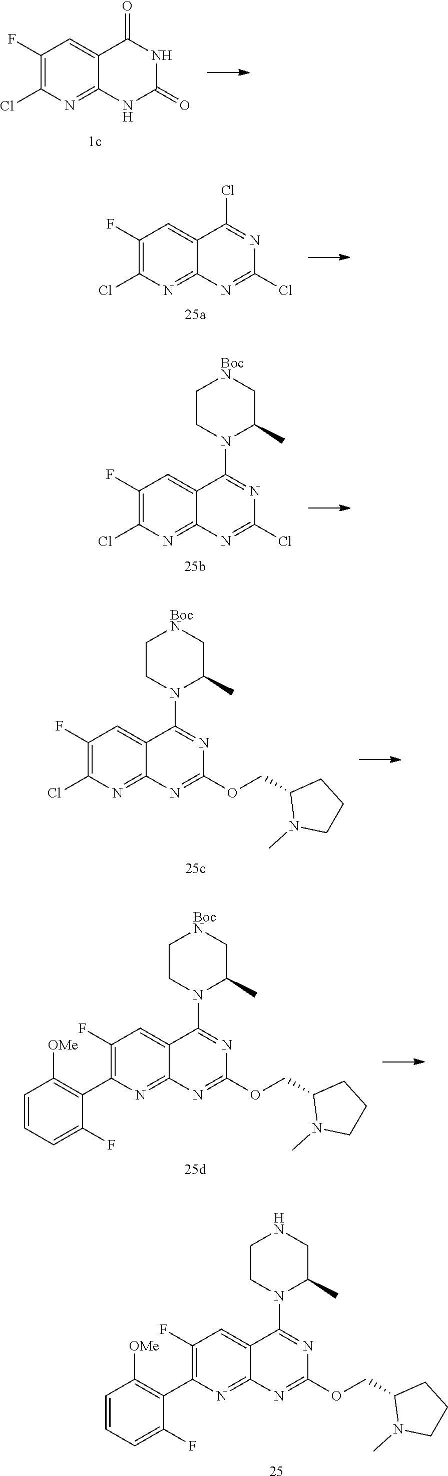

### Step 1: 2,4,7-trichloro-6-fluoropyrido[2,3-d]pyrimidine

To a solution of 7-chloro-6-fluoropyrido[2,3-d]pyrimidine-2,4(1H,3H)-dione 1c (18.3 g, 85 mmol) in POCl₃ (200 mL) was added N,N-Dimethylaniline (10 mL). The resulting solution was heated to reflux and stirred for 10 h. The mixture was poured into ice water, precipitated a large amount of yellow solid, filtered, filter cake washed with water, dried under vacuum to afford the crude compound as a yellow solid (18.0 g, 84%). LC/MS(ESI): m/z=253[M+H]⁺.

### Step 2: (R)-tert-butyl 4-(2,7-dichloro-6-fluoropyrido[2,3-d]pyrimidin-4-yl)-3-methyl piperazine-1-carboxylate

To a solution of 2,4,7-trichloro-6-fluoropyrido[2,3-d]pyrimidine 25a (12.6 g, 50 mmol), (R)-tert-butyl 3-methylpiperazine-1-carboxylate (11 g, 55 mmol) in DMF (300 mL) was added K₂CO₃ (10.35 g, 75 mmol) and catalytic amount KI. The mixture was heated to 120° C. and stirred for 4 h. After cooling to room temperature, the mixture was diluted with water, extrated with ethyl acetate, and washed with water and saturated brine solution. The organic layer was dried over anhydrous sodium sulfate, filtered and concentrated. The residue was purified by flash column to afford the title compound as a yellow solid (16.2 g, 78%). LC/MS(ESI): m/z=417[M+H]⁺.

### Step 3: (R)-tert-butyl 4-(7-chloro-6-fluoro-2-(((S)-1-methylpyrrolidin-2-yl)methoxy) pyrido[2,3-d]pyrimidin-4-yl)-3-methylpiperazine-1-carboxylate

To a solution of (R)-tert-butyl 4-(2,7-dichloro-6-fluoropyrido[2,3-d]pyrimidin-4-yl)-3-methyl

piperazine-1-carboxylate 25b (12.5 g, 30 mmol), (S)-(1-methylpyrrolidin-2-yl)methanol (3.9 g, 33 mmol) in DMF (100 mL) was added K₂CO₃ (6.2 g, 45 mmol) and catalytic amount KI. The mixture was heated to 120° C. and stirred for 4 h. After cooling to room temperature, the mixture was diluted with water, extrated with ethyl acetate, and washed with water and saturated brine solution. The organic layer was dried over anhydrous sodium sulfate, filtered and concentrated. The residue was purified by flash column to afford the title compound as a yellow solid (10.24 g, 69%). LC/MS(ESI): m/z=496[M+H]⁺.

### Step 4: (3R)-tert-butyl 4-(6-fluoro-7-(2-fluoro-6-methoxyphenyl)-2-(((S)-1-methyl pyrrolidin-2-yl)methoxy)pyrido[2,3-d]pyrimidin-4-yl)-3-methylpiperazine-1-carboxylate

To a solution of (R)-tert-butyl 4-(7-chloro-6-fluoro-2-(((S)-1-methylpyrrolidin-2-yl)methoxy)

pyrido[2,3-d]pyrimidin-4-yl)-3-methylpiperazine-1-carboxylate 25c (496 mg, 1 mmol), Cs₂CO₃ (652 mg, 2 mmol) and (2-fluoro-6-methoxyphenyl)boronic acid (170 mg, 1 mmol) in 1,4-dioxane (10 mL) and water (2 mL) was added Pd₂(dba)₃ (80 mg, 0.088 mmol) under nitrogen atmosphere, and the mixture was degassed with nitrogen 3 times, then heated to reflux and stirred for 16 h. The solution was cooled to room temperature and stirred overnight, water (2 mL) was added then filtered and concentrated. The residue was slurryed with MeOH to afford the title compound as an off-white solid (397 mg, 68%). LC/MS(ESI): m/z=585.3[M+H]⁺.

### Step 5: 6-fluoro-7-(2-fluoro-6-methoxyphenyl)-4-((R)-2-methylpiperazin-1-yl)-2-(((S)-1-methylpyrrolidin-2-yl)methoxy)pyrido[2,3-d]pyrimidine

To a solution of (3R)-tert-butyl 4-(6-fluoro-7-(2-fluoro-6-methoxyphenyl)-2-(((S)-1-methyl

pyrrolidin-2-yl)methoxy)pyrido[2,3-d]pyrimidin-4-yl)-3-methylpiperazine-1-carboxylate 25d (292 mg, 0.5 mmol) in EtOAc (2 mL) was added hydrogen chloride (4 mL, 1.0 M solution in 1,4-dioxane). The reaction was stirred for 2 h at room temperature. The solution was neutralized with 1N aq. NaOH, extracted with ethyl acetate. The organic layer was washed with saturated aqueous bicarbonate and dried over anhydrous sodium sulfate, filtered and concentrated to afford the title compound (183 mg, 76%). LC/MS(ESI): m/z=485.2[M+H]⁺.

The following Compounds were synthesized using the above procedure or modification procedure (Compound 25) using the corresponding Intermediate Examples 26-44 refer to the preparation method of compound 25 and its corresponding intermediate.

| Com - | | | MS:(M + H)⁺/ |
|---|---|---|---|
| pound | Chemical Name | Structure | ¹H NMR |
| 26 | (5-(6-fluoro-4- ((R)-2- methylpiperazin-1- yl)-2-(((S)-1- methylpyrrolidin- 2- yl)methoxy)pyrido [2,3-d]pyrimidin- 7-yl)-2- methoxyphenyl) methanol | 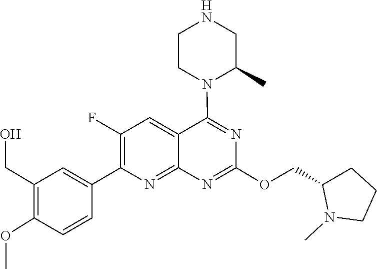 [SMILES: COc1ccc(-c2nc3nc(OC[C@@H]4CCCN4C)nc(N4CCNC[C@H]4C)c3cc2F)cc1CO] | LC/MS(ESD): m/z = 497.3 [M + H]⁺ |
| 27 | 3-chloro-2-(6- fluoro-4-((R)-2- methylpiperazin-1- y1)-2-(((S)-1- methylpyrrolidin- 2- yl)methoxy)pyrido [2,3-d]pyrimidin- 7-yl)phenol | 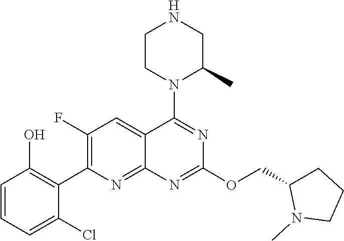 [SMILES: C[C@@H]1CNCCN1c1nc(OC[C@@H]2CCCN2C)nc2nc(-c3c(O)cccc3Cl)c(F)cc12] | LC/MS(ESI): m/z = 488.3 [M + H]⁺ |
| 28 | 6-fluoro-4-((R)-2- methylpiperazin-1- y1)-2-(((S)-1- methylpyrrolidin- 2-yl)methoxy)-7- (2- (trifluoromethyl) phenyl)pyrido[2,3- d]pyrimidine | 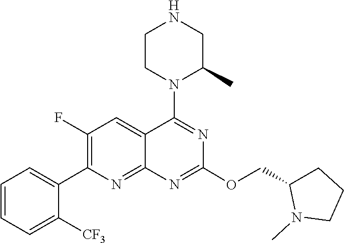 [SMILES: C[C@@H]1CNCCN1c1nc(OC[C@@H]2CCCN2C)nc2nc(-c3ccccc3C(F)(F)F)c(F)cc12] | LC/MS(ESI): m/z = 505.3 [M + H]⁺ |
| 29 | 6-fluoro-7-(3- fluoropyridin-4- yl)-4-((R)-2- methylpiperazin-1- y1)-2-(((S)-1- methylpyrrolidin- 2- yl)methoxy)pyrido [2,3-d]pyrimidine | 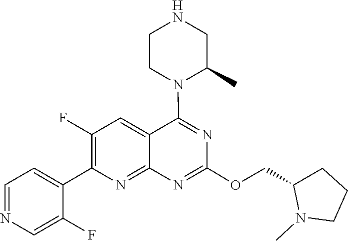 [SMILES: C[C@@H]1CNCCN1c1nc(OC[C@@H]2CCCN2C)nc2nc(-c3ccncc3F)c(F)cc12] | LC/MS(ESI): m/z = 456.2 [M + H]⁺ |
| 30 | 7-(3-chloropyridin- 4-yl)-6-fluoro-4- ((R)-2- methylpiperazin-1- y1)-2-(((S)-1- methylpyrrolidin- 2- yl)methoxy)pyrido [2,3-d]pyrimidine | 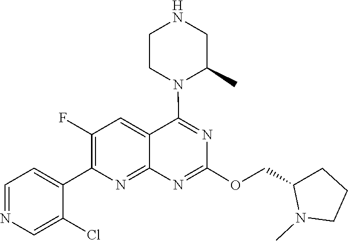 [SMILES: C[C@@H]1CNCCN1c1nc(OC[C@@H]2CCCN2C)nc2nc(-c3ccncc3Cl)c(F)cc12] | LC/MS(ESI): m/z = 473.2 [M + H]⁺ |
| 31 | 6-fluoro-4-((R)-2- methylpiperazin-1- y1)-2-(((S)-1- methylpyrrolidin- 2-yl)methoxy)-7- (3- (trifluoromethyl) pyridin-4- yl)pyrido[2,3- d]pyrimidine | 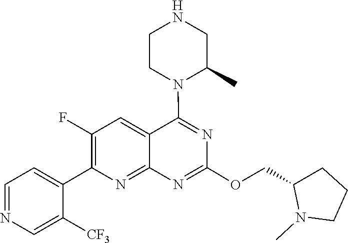 [SMILES: C[C@@H]1CNCCN1c1nc(OC[C@@H]2CCCN2C)nc2nc(-c3ccncc3C(F)(F)F)c(F)cc12] | LC/MS(ESI): m/z = 506.2 [M + H]⁺ |
| 32 | 7-(2- chlorophenyl)-6- fluoro-4-((R)-2- methylpiperazin-1- yl)-2-(((S)-1- methylpyrrolidin- 2- yl)methoxy)pyrido [2,3-d]pyrimidine | 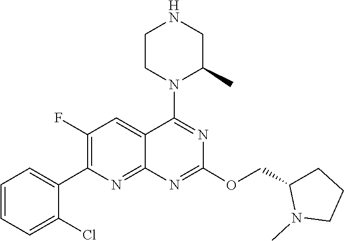 [SMILES: C[C@@H]1CNCCN1c1nc(OC[C@@H]2CCCN2C)nc2nc(-c3ccccc3Cl)c(F)cc12] | LC/MS(ESI): m/z = 472.2 [M + H]⁺ |
| 33 | 6-fluoro-7-(8- fluoronaphthalen- 1-y1)-4-((R)-2- methylpiperazin-1- y1)-2-(((S)-1- methylpyrrolidin- 2- yl)methoxy)pyrido [2,3-d]pyrimidine | 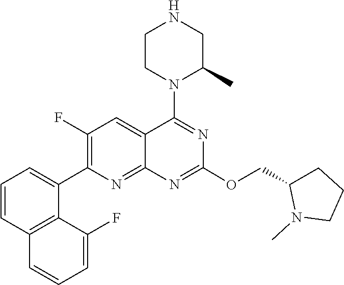 [SMILES: C[C@@H]1CNCCN1c1nc(OC[C@@H]2CCCN2C)nc2nc(-c3cccc4cccc(F)c34)c(F)cc12] | LC/MS(ESI): m/z = 505.3 [M + H]⁺ |
| 34 | 7-(8- chloronaphthalen- 1-yl)-6-fluoro-4- ((R)-2- methylpiperazin-1- yl)-2-(((S)-1- methylpyrrolidin- 2- yl)methoxy)pyrido [2,3-d]pyrimidine | 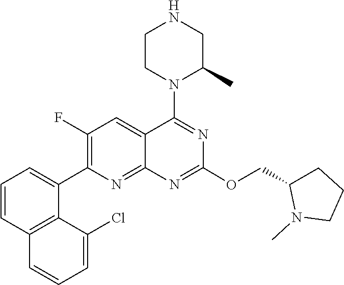 [SMILES: C[C@@H]1CNCCN1c1nc(OC[C@@H]2CCCN2C)nc2nc(-c3cccc4cccc(Cl)c34)c(F)cc12] | LC/MS(ESI): m/z = 522.3 [M + H]⁺ |
| 35 | 6-fluoro-4-((R)-2- methylpiperazin-1- yl)-2-(((S)-1- methylpyrrolidin- 2-yl)methoxy)-7- (naphthalen-1- yl)pyrido[2,3- d]pyrimidine | 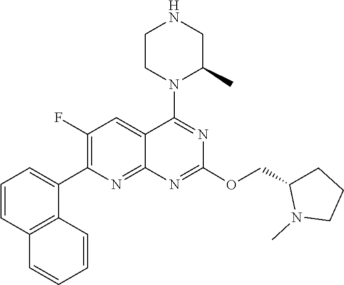 [SMILES: C[C@@H]1CNCCN1c1nc(OC[C@@H]2CCCN2C)nc2nc(-c3cccc4ccccc34)c(F)cc12] | LC/MS(ESI): m/z = 487.3 [M + H]⁺ |
| 36 | 4-(6-fluoro-4-((R)- 2-methylpiperazin- 1-yl)-2-(((S)-1- methylpyrrolidin- 2- yl)methoxy)pyrido [2,3-d]pyrimidin- 7-yl)naphthalen-2- ol | 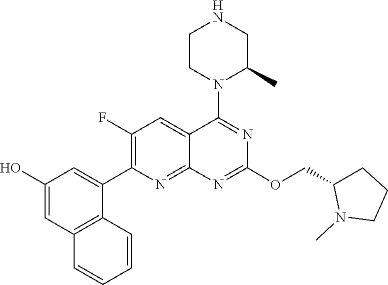 [SMILES: C[C@@H]1CNCCN1c1nc(OC[C@@H]2CCCN2C)nc2nc(-c3cc(O)cc4ccccc34)c(F)cc12] | LC/MS(ESI): m/z = 503.3 [M + H]⁺ |
| 37 | 6-fluoro-4-((R)-2- methylpiperazin-1- y1)-2-(((S)-1- methylpyrrolidin- 2-yl)methoxy)-7- (naphthalen-2- yl)pyrido[2,3- d]pyrimidine | 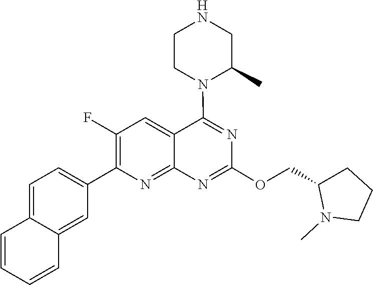 [SMILES: C[C@@H]1CNCCN1c1nc(OC[C@@H]2CCCN2C)nc2nc(-c3ccc4ccccc4c3)c(F)cc12] | LC/MS(ESI): m/z = 487.3 [M + H]⁺ |
| 38 | 7- (benzo[b]thiophen- 7-yl)-6-fluoro-4- ((R)-2- methylpiperazin-1- y1)-2-(((S)-1- methylpyrrolidin- 2- yl)methoxy)pyrido [2,3-d]pyrimidine | 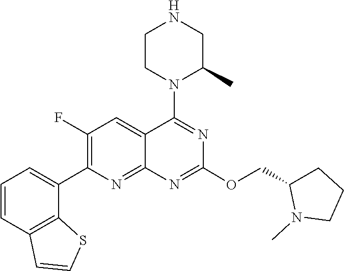 [SMILES: C[C@@H]1CNCCN1c1nc(OC[C@@H]2CCCN2C)nc2nc(-c3cccc4ccsc34)c(F)cc12] | LC/MS(ESD): m/z = 493.3 [M + H]⁺ |
| 39 | 6-fluoro-4-((R)-2- methylpiperazin-1- yl)-2-(((S)-1- methylpyrrolidin- 2-yl)methoxy)-7- (quinolin-8- yl)pyrido[2,3- d]pyrimidine | 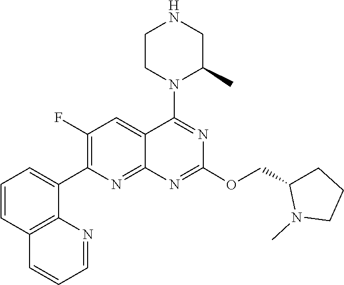 [SMILES: C[C@@H]1CNCCN1c1nc(OC[C@@H]2CCCN2C)nc2nc(-c3cccc4cccnc34)c(F)cc12] | LC/MS(ESD): m/z = 488.3 [M + H]⁺ |
| 40 | 7-(benzofuran-7- yl)-6-fluoro-4- ((R)-2- methylpiperazin-1- yl)-2-(((S)-1- methylpyrrolidin- 2- yl)methoxy)pyrido [2,3-d]pyrimidine | 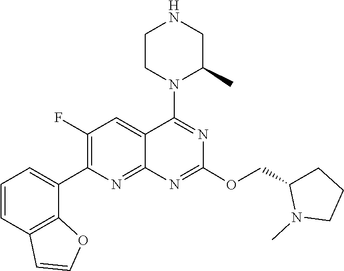 [SMILES: C[C@@H]1CNCCN1c1nc(OC[C@@H]2CCCN2C)nc2nc(-c3cccc4ccoc34)c(F)cc12] | LC/MS(ESD): m/z = 477.3 [M + H]⁺ |
| 41 | 6-fluoro-7-(1- methyl-1H-indol- 4-yl)-4-((R)-2- methylpiperazin-1- yl)-2-(((S)-1- methylpyrrolidin- 2- yl)methoxy)pyrido [2,3-d]pyrimidine | 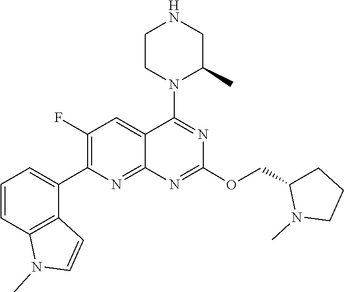 [SMILES: C[C@@H]1CNCCN1c1nc(OC[C@@H]2CCCN2C)nc2nc(-c3cccc4c3ccn4C)c(F)cc12] | LC/MS(ESI): m/z = 491.2 [M + H]⁺ |
| 42 | 6-fluoro-7-(1- methyl-1H- indazol-4-yl)-4- ((R)-2- methylpiperazin-1- yl)-2-(((S)-1- methylpyrrolidin- 2- yl)methoxy)pyrido [2,3-d]pyrimidine | 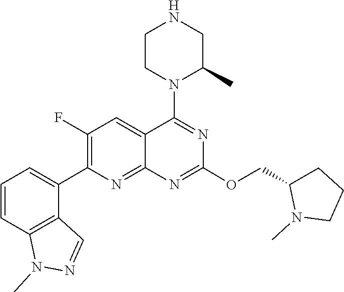 [SMILES: C[C@@H]1CNCCN1c1nc(OC[C@@H]2CCCN2C)nc2nc(-c3cccc4c3cnn4C)c(F)cc12] | LC/MS(ESI): m/z = 490.3 [M + H]⁺ |
| 43 | 6-fluoro-7- (isoquinolin-8-y1)- 4-((R)-2- methylpiperazin-1- y1)-2-(((S)-1- methylpyrrolidin- 2- yl)methoxy)pyrido [2,3-d]pyrimidine | 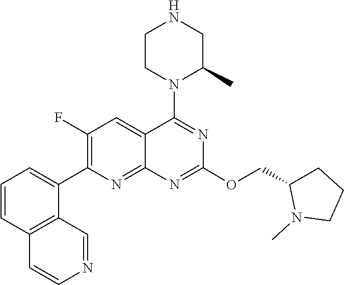 [SMILES: C[C@@H]1CNCCN1c1nc(OC[C@@H]2CCCN2C)nc2nc(-c3cccc4ccncc34)c(F)cc12] | LC/MS(ESI): m/z = 488.3 [M + H]⁺ |
| 44 | 6-fluoro-4-((R)-2- methylpiperazin-1- y1)-2-(((S)-1- methylpyrrolidin- 2-yl)methoxy)-7- (quinolin-5- yl)pyrido[2,3- d]pyrimidine | 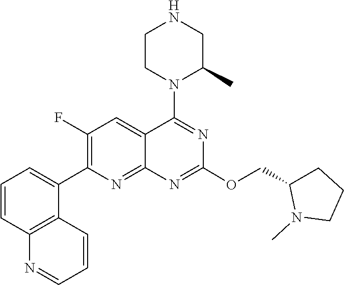 [SMILES: C[C@@H]1CNCCN1c1nc(OC[C@@H]2CCCN2C)nc2nc(-c3cccc4ncccc34)c(F)cc12] | LC/MS(ESI): m/z = 488.3 [M + H]⁺ |
| 45 | 5-chloro-4-(6- fluoro-4-((R)-2- methylpiperazin-1- yl)-2-(((S)-1- methylpyrrolidin- 2- yl)methoxy)pyrido [2,3-d]pyrimidin- 7-yl)naphthalen-2- ol | 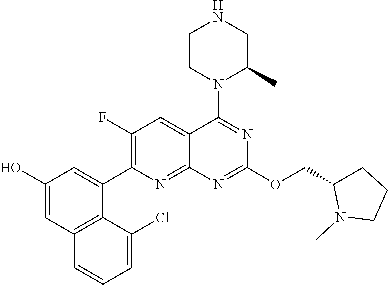 [SMILES: C[C@@H]1CNCCN1c1nc(OC[C@@H]2CCCN2C)nc2nc(-c3cc(O)cc4cccc(Cl)c34)c(F)cc12] | LC/MS(ESI): m/z = 538.3 [M + H]⁺ |
| 46 | 5-fluoro-4-(6- fluoro-4-((R)-2- methylpiperazin-1- yl)-2-(((S)-1- methylpyrrolidin- 2- yl)methoxy)pyrido [2,3-d]pyrimidin- 7-yl)naphthalen-2- ol | 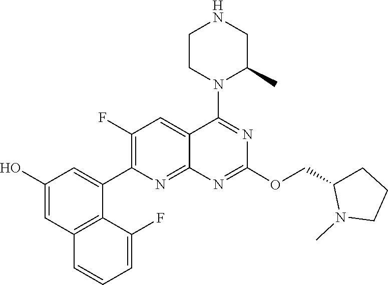 [SMILES: C[C@@H]1CNCCN1c1nc(OC[C@@H]2CCCN2C)nc2nc(-c3cc(O)cc4cccc(F)c34)c(F)cc12] | LC/MS(ESI): mz = 521.3 [M + H]⁺ |
| 47 | 4-(4-(3,6- diazabicyclo[3.1.1] heptan-6-yl)-6- fluoro-2-(((S)-1- methylpyrrolidin- 2- yl)methoxy)pyrido [2,3-d]pyrimidin- 7-yl)-5- chloronaphthalen- 2-ol | 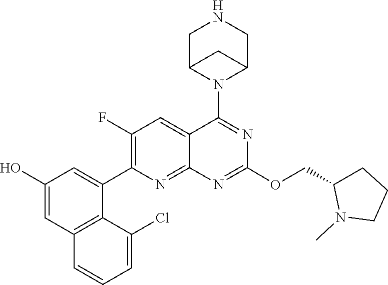 [SMILES: CN1CCC[C@H]1COc1nc(N2C3CNCC2C3)c2cc(F)c(-c3cc(O)cc4cccc(Cl)c34)nc2n1] | LC/MS(ESI): m/z = 536.3 [M + H]⁺ |
| 48 | 4-(4-(3,6- diazabicyclo[3.1.1] heptan-6-yl)-6- fluoro-2-(((S)-1- methylpyrrolidin- 2- yl)methoxy)pyrido [2,3-d]pyrimidin- 7-yl)-5- fluoronaphthalen- 2-ol | 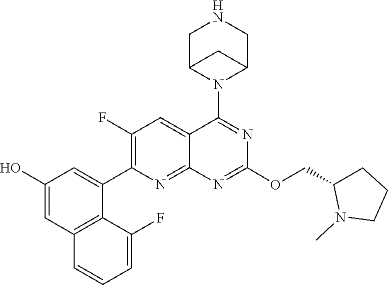 [SMILES: CN1CCC[C@H]1COc1nc(N2C3CNCC2C3)c2cc(F)c(-c3cc(O)cc4cccc(F)c34)nc2n1] | LC/MS(ESI): m/z = 519.2 [M + H]⁺ |
| 49 | 4-(4-(3,6- diazabicyclo[3.1.1] heptan-6-yl)-6- fluoro-2-(((2S,4R)- 4-fluoro-1- methylpyrrolidin- 2- yl)methoxy)pyrido [2,3-d]pyrimidin- 7-y1)-5- fluoronaphthalen- 2-ol | 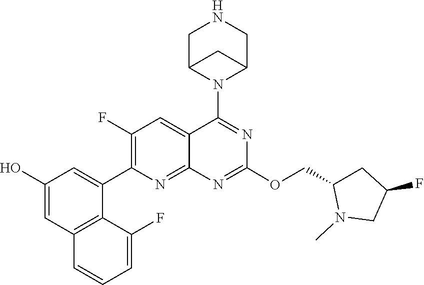 [SMILES: CN1C[C@H](F)C[C@H]1COc1nc(N2C3CNCC2C3)c2cc(F)c(-c3cc(O)cc4cccc(F)c34)nc2n1] | LC/MS(ESI): m/z = 537.2 [M + H]⁺ |
| 50 | 4-(4-(3,6- diazabicyclo[3.1.1] heptan-6-yl)-2- (((2S,4R)-1- cyclopropyl-4- fluoropyrrolidin-2- yl)methoxy)-6- fluoropyrido[2,3- d]pyrimidin-7-yl)- 5- fluoronaphthalen- 2-ol | 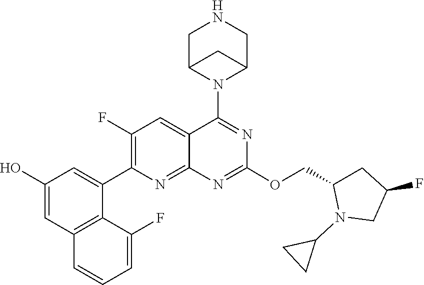 [SMILES: Oc1cc(-c2nc3nc(OC[C@@H]4C[C@@H](F)CN4C4CC4)nc(N4C5CNCC4C5)c3cc2F)c2c(F)cccc2c1] | LC/MS(ESI): m/z = 563.2 [M + H]⁺ |
| 51 | 4-(4-(3,6- diazabicyclo[3.1.1] heptan-6-yl)-6- fluoro-2-((1- (pyrrolidin-1- ylmethyl)cyclopro pyl)methoxy)pyrid o[2,3-d]pyrimidin- 7-yl)-5- fluoronaphthalen- 2-ol | 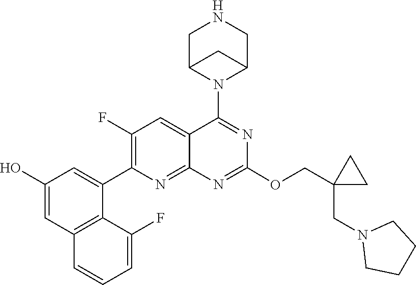 [SMILES: Oc1cc(-c2nc3nc(OCC4(CN5CCCC5)CC4)nc(N4C5CNCC4C5)c3cc2F)c2c(F)cccc2c1] | LC/MS (ESI): m/z = 559.3 [M + H]⁺ |
| 52 | 4-(4-(3,6- diazabicyclo[3.1.1] heptan-6-yl)-6- fluoro-2-((1-(((R)- 3-fluoropyrrolidin- 1- yl)methyl)cyclopro pyl)methoxy)pyrid o[2,3-d]pyrimidin- 7-yl)-5- fluoronaphthalen- 2-ol | 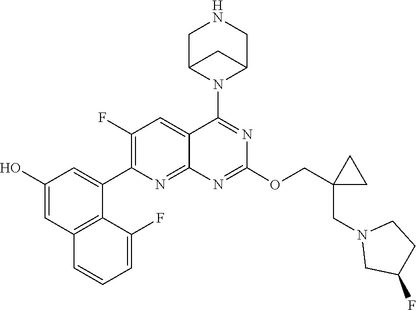 [SMILES: Oc1cc(-c2nc3nc(OCC4(CN5CC[C@@H](F)C5)CC4)nc(N4C5CNCC4C5)c3cc2F)c2c(F)cccc2c1] | LC/MS(ESI): m/z = 577.3 [M + H]⁺ |

### Example 53

### 4-(4-(3,6-diazabicyclo[3.1.1]heptan-6-yl)-6-fluoro-2-(((2R,7aS)-2-fluorohexahydro-1H-pyrrolizin-7a-yl)methoxy)pyrido[2,3-d]pyrimidin-7-yl)-5-chloronaphthalen-2-ol

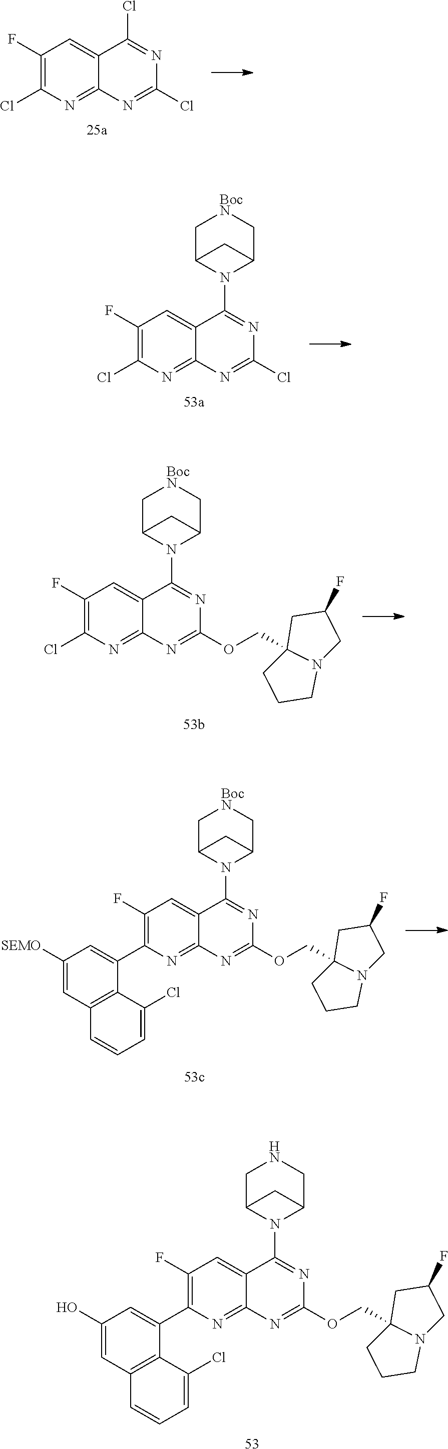

### Step 1: tert-butyl 6-(2,7-dichloro-6-fluoropyrido[2,3-d]pyrimidin-4-yl)-3,6-diazabicyclo[3.1.1]heptane-3-carboxylate

To a solution of 2,4,7-trichloro-6-fluoropyrido[2,3-d]pyrimidine 25a (2.52 g, 10 mmol), tert-butyl 3,6-diazabicyclo[3.1.1]heptane-3-carboxylate (2.18 g, 11 mmol) in DMF (60 mL) was added K₂CO₃ (2.07 g, 15 mmol) and catalytic amount KI. The mixture was heated to 120° C. and stirred for 4 h. After cooling to room temperature, the mixture was diluted with water, extrated with ethyl acetate, and washed with water and saturated brine solution. The organic layer was dried over anhydrous sodium sulfate, filtered and concentrated. The residue was purified by flash column to afford the title compound as a yellow solid (3.81 g, 74%). LC/MS(ESI): m/z=515[M+H]⁺.

### Step 2: tert-butyl 6-(7-chloro-6-fluoro-2-(((2R,7aS)-2-fluorohexahydro-1H-pyrrolizin-7a-yl)methoxy)pyrido[2,3-d]pyrimidin-4-yl)-3,6-diazabicyclo[3.1.1]heptane-3-carboxylate

To a solution of tert-butyl 6-(2,7-dichloro-6-fluoropyrido[2,3-d]pyrimidin-4-yl)-3,6-diaza

bicyclo[3.1.1]heptane-3-carboxylate 53a (250 mg, 1 mmol), ((2R,7aS)-2-fluorohexahydro-1H-pyrrolizin-7a-yl)methanol (148 mg, 1.1 mmol) in DMF (8 mL) was added K₂CO₃ (193 mg, 1.4 mmol) and catalytic amount KI. The mixture was heated to 120° C. and stirred for 4 h. After cooling to room temperature, the mixture was diluted with water, extrated with ethyl acetate, and washed with water and saturated brine solution. The organic layer was dried over anhydrous sodium sulfate, filtered and concentrated. The residue was purified by flash column to afford the title compound as a yellow solid (387 mg, 72%). LC/MS(ESI): m/z=538.2[M+H]⁺.

### Step 3: tert-butyl 6-(7-(8-chloro-3-((2-(trimethylsilyl)ethoxy)methoxy)naphthalen-1-yl)-6-fluoro-2-(((2R,7aS)-2-fluorohexahydro-1H-pyrrolizin-7a-yl)methoxy)pyrido[2,3-d]pyrimidin-4-yl)-3,6-diazabicyclo[3.1.1]heptane-3-carboxylate

To a solution of tert-butyl (7-chloro-6-fluoro-2-(((2R,7aS)-2-fluorohexahydro-1H-pyrrolizin-7a-yl)methoxy)pyrido[2,3-d]pyrimidin-4-yl)-3,6-diazabicyclo[3.1.1 ]heptane-3-carboxylate(107 mg, 0.2 mmol), Cs₂CO₃ (130 mg, 0.4 mmol) and (2-(((5-chloro-4-(4,4,5,5-tetramethyl-1,3,2-

dioxaborolan-2-yl)naphthalen-2-yl)oxy)methoxy)ethyl)trimethylsilane (87 mg, 0.2 mmol) in 1,4-dioxane (5 mL) and water (1 mL) was added Pd₂(dba)₃ (20 mg, 0.022 mmol) under nitrogen atmosphere, then heated to reflux and stirred for 16 h. The solution was cooled to room temperature and stirred overnight, water (2 mL) was added then filtered and concentrated. The residue was slurryed with MeOH to afford the title compound as an off-white solid (94 mg, 58%). LC/MS(ESI): m/z=809.3[M+H]⁺.

### Step 4: 4-(4-(3,6-diazabicyclo[3.1.1]heptan-6-yl)-6-fluoro-2-(((2R,7aS)-2-fluorohexa hydro-1H-pyrrolizin-7a-yl)methoxy)pyrido[2,3-d]pyrimidin-7-yl)-5-chloronaphthalen-2-ol

tert-butyl 6-(7-(8-chloro-3-((2-(trimethylsilyl)ethoxy)methoxy)naphthalen-1-yl)-6-fluoro-2-(((2R, 7aS)-2-fluorohexahydro-1H-pyrrolizin-7a- yl)methoxy)pyrido[2,3-d]pyrimidin-4-yl)-3,6-diazabicyclo[3.1.1]heptane-3-carboxylate (81 mg, 0.1 mmol) was dissolved in TFA (3 mL). The solution was stirred for 2 h at room temperature. The solution was concentrated under reduced pressure and the residue was purified by Pre-HPLC to afford the title compound (32 mg, 56%) as a light solid. LC/MS(ESI): m/z=579.2[M+H]⁺.

Examples 54-56 refer to the preparation method of compound 25 and its corresponding intermediate.

| | Chemical | | MS:(M + H)⁺/ |
|---|---|---|---|
| Compound | Name | Structure | ¹H NMR |
| 54 | 4-(4-(3,6- diazabicyclo [3.1.1 ]heptan-6- y1)-6-fluoro-2- (((2R,7aS)-2- fluorohexahydro- 1H-pyrrolizin- 7a-yl)methoxy) pyrido[2,3- d]pyrimidin-7- y1)-5- fluoronaphthalen- 2-ol | 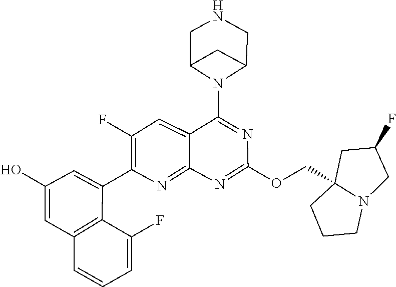 [SMILES: Oc1cc(-c2nc3nc(OC[C@@]45CCCN4C[C@H](F)C5)nc(N4C5CNCC4C5)c3cc2F)c2c(F)cccc2c1] | LC/MS(ESI): m/z = 563.2 [M + H]⁺ |
| 55 | 4-(4-((1R,5S)- 3,8- diazabicyclo [3.2.1]octan-3- yl)-6-fluoro-2- (((2R,7aS)-2- fluorohexahydro- 1H-pyrrolizin- 7a-yl)methoxy) pyrido[2,3- d]pyrimidin-7- yl)-5- chloronaphthalen- 2-ol | 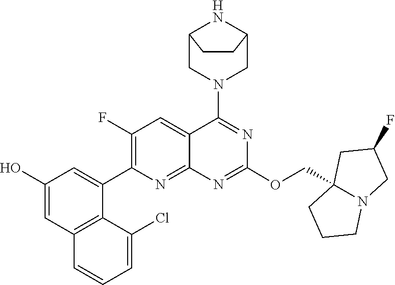 [SMILES: Oc1cc(-c2nc3nc(OC[C@@]45CCCN4C[C@H](F)C5)nc(N4CC5CCC(C4)N5)c3cc2F)c2c(Cl)cccc2c1] | LC/MS(ESI): m/z = 593.2 [M + H]⁺ |
| 56 | 4-(4-((1R,5S)- 3,8- diazabicyclo [3.2.1]octan-3- yl)-6-fluoro-2- (((2R,7aS)-2- fluorohexahydro- 1H- pyrrolizin-7a- yl)methoxy) pyrido[2,3- d]pyrimidin-7- y1)-5- fluoronaphthalen- 2-ol | 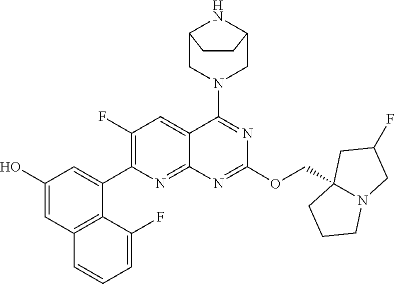 [SMILES: Oc1cc(-c2nc3nc(OC[C@@]45CCCN4CC(F)C5)nc(N4CC5CCC(C4)N5)c3cc2F)c2c(F)cccc2c1] | LC/MS(ESI): m/z = 577.3 [M + H]⁺ |

### Example 57

### 4-(5-((1R,5S)-3,8-diazabicyclo[3.2.1]octan-3-yl)-3-fluoro-7-(((2R,7aS)-2-fluorohexahydro-1H-pyrrolizin-7a-yl)methoxy)-1,8-naphthyridin-2-yl)-5-fluoronaphthalen-2-ol

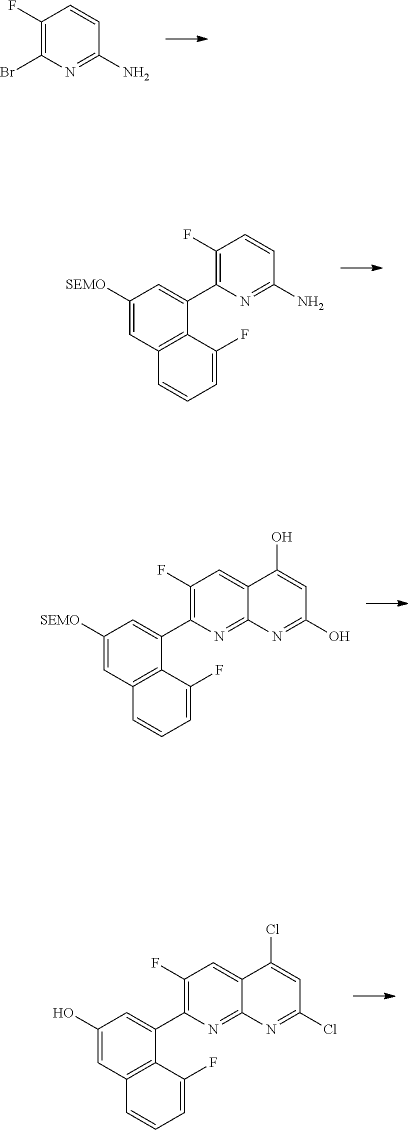

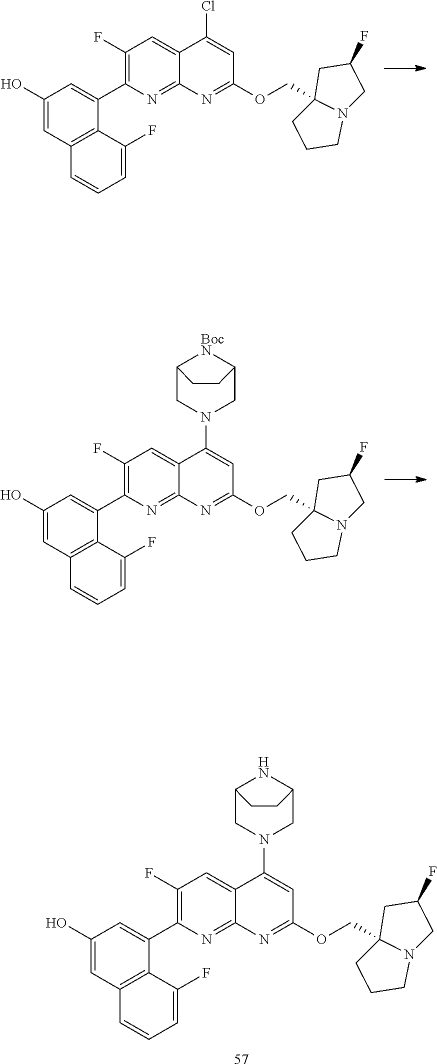 [SMILES: Oc1cc(-c2nc3nc(OC[C@@]45CCCN4C[C@H](F)C5)cc(Cl)c3cc2F)c2c(F)cccc2c1 ; CC(C)(C)OC(=O)N1C2CCC1CN(c1cc(OC[C@@]34CCCN3C[C@H](F)C4)nc3nc(-c4cc(O)cc5cccc(F)c45)c(F)cc13)C2 ; Oc1cc(-c2nc3nc(OC[C@@]45CCCN4C[C@H](F)C5)cc(N4CC5CCC(C4)N5)c3cc2F)c2c(F)cccc2c1 ; "57"]

### Step 1: 5-fluoro-6-(8-fluoro-3-((2-(trimethylsilyl)ethoxy)methoxy)naphthalen-1-yl) pyridin-2-amine

To a solution of 6-bromo-5-fluoropyridin-2-amine (95 mg, 0.5 mmol), Cs₂CO₃ (326 mg, 1 mmol) and (2-(((5-fluoro-4-(4,4,5,5-tetramethyl-1,3,2-dioxaborolan-2-yl)naphthalen-2-yl)oxy)

methoxy)ethyl)trimethylsilane (209 mg, 0.5 mmol) in 1,4-dioxane (5 mL) and water (1 mL) was added Pd₂(dba)₃ (40 mg, 0.044 mmol) under nitrogen atmosphere, then heated to reflux and stirred for 16 h. The solution was cooled to room temperature and stirred overnight, water (1 mL) was added then filtered. The to afford the title compound as an off-white solid (153 mg, 76%). LC/MS(ESI): m/z=403.2[M+H]⁺.

### Step 2: 6-fluoro-7-(8-fluoro-3-((2-(trimethylsilyl)ethoxy)methoxy)naphthalen-1-yl)-1,8-naphthyridine-2,4-diol

5-fluoro-6-(8-fluoro-3-((2-(trimethylsilyl)ethoxy)methoxy)naphthalen-1-yl)pyridin-2-amine (121 mg, 0.3 mmol) and diethyl malonate (55.1 mg, 0.33 mmol) were suspended in diphenyl ether (5 mL), the reaction was heated at 150° C.for 0.5 h where upon the mixture became a homogeneous solution. The reaction was then refluxed for 2 h, then cooled to room temperature, poured into water and extracted with ethyl acetate. The organic phase was dried over anhydrous magnesium sulphate, filtered and concentrated. The residue was heated at 220° C.under reduced pressure for 2 h where the mixture solidified. The reaction was cooled to room temperature and the solids were washed with ethyl alcohol and filtered to give the title compound as a yellow solid (96 mg, 68%). LC/MS(ESD): m/z=471.2[M+H]⁺

### Step 3: 4-(5,7-dichloro-3-fluoro-1,8-naphthyridin-2-yl)-5-fluoronaphthalen-2-ol

To a solution of 6-fluoro-7-(8-fluoro-3-((2-(trimethylsilyl)ethoxy)methoxy)naphthalen-1-yl)-1,8-naphthyridine-2,4-diol (95 mg, 0.2 mmol) in POCl₃ (2 mL) and toluene (5 mL) was added trace N,N-Dimethylaniline. The resulting solution was heated to reflux and stirred for 10 h. The mixture was poured into ice water, filtered, filter cake washed with water, dried under vacuum to afford the crude compound as a yellow solid (67 mg, 87%). LC/MS(ESD): m/z=378[M+H]⁺.

### Step 4: 4-(5-chloro-3-fluoro-7-(((2R,7aS)-2-fluorohexahydro-1H-pyrrolizin-7a-yl) methoxy)-1,8-naphthyridin-2-yl)-5-fluoronaphthalen-2-ol

To a solution of 4-(5,7-dichloro-3-fluoro-1,8-naphthyridin-2-yl)-5-fluoronaphthalen-2-ol (66 mg, 0.175 mmol), ((2R,7aS)-2-fluorohexahydro-1H-pyrrolizin-7a-yl)methanol (31 mg, 0.193 mmol) in DMF (8 mL) was added K₂CO₃ (48 mg, 0.35 mmol) and catalytic amount KI. The mixture was heated to 120° C. and stirred for 4 h. After cooling to room temperature, the mixture was concentrated. The residue was purified by flash column to afford the title compound as a yellow solid (68 mg, 78%). LC/MS(ESI): m/z=501.2[M+H]⁺.

### Step 5: (3R)-tert-butyl 4-(6-fluoro-7-(2-fluoro-6-methoxyphenyl)-2-(((S)-1-methyl pyrrolidin-2-yl)methoxy)-1,8-naphthyridin-4-yl)-3-methylpiperazine-1-carboxylate

To a solution of 4-(5-chloro-3-fluoro-7-(((2R,7aS)-2-fluorohexahydro-1H-pyrrolizin-7a-yl)

methoxy)-1,8-naphthyridin-2-yl)-5-fluoronaphthalen-2-ol (67 mg, 0.134 mmol), tert-butyl 3,8-diazabicyclo[3.2.1 ]octane-8-carboxylate (31.8 mg, 0.15 mmol), Cs₂CO₃ (87 mg, 0.268 mmol) and BINAP (4 mg, 0.0067 mmol) in 1,4-dioxane (2 mL) was added Pd₂(dba)₃ (10 mg, 0.01 mmol) under nitrogen atmosphere, and the mixture was degassed with nitrogen 3 times, then heated to reflux and stirred for 24 h. After cooling to room temperature, the mixture was diluted with ethyl acetate, washed with water and saturated brine solution. The organic layer was dried over anhydrous sodium sulfate, filtered and concentrated. The residue was was purified by Pre-HPLC to afford the title compound as a yellow solid (74 mg, 82%). LC/MS(ESI): m/z=676.3[M+H]⁺.

### Step 6: 3-fluoro-2-(3-fluoro-5-((R)-2-methylpiperazin-1-yl)-7-(((S)-1-methylpyrrolidin 2-yl)methoxy)-1,8-naphthyridin-2-yl)phenol

To a solution of (3R)-tert-butyl 4-(6-fluoro-7-(2-fluoro-6-methoxyphenyl)-2-(((S)-1-methyl

pyrrolidin-2-yl)methoxy)-1,8-naphthyridin-4-yl)-3-methylpiperazine-1-carboxylate (72 mg, 0.1 mmol) in EtOAc (1 mL) was added hydrogen chloride (1 mL, 1.0 M solution in 1,4-dioxane). The reaction was stirred for 2 h at room temperature. The solution was neutralized with 1N aq. NaOH, extracted with ethyl acetate. The organic layer was washed with saturated aqueous bicarbonate and dried over anhydrous sodium sulfate, filtered and concentrated. The residue was was purified by Pre-HPLC to afford the title compound as a yellow solid (50 mg, 87%). L.C/MS(ESI): m/z=576.2[M+H]⁺.

### Example 58

### 4-(4-((1R,5S)-3,8-diazabicyclo[3.2.1 ]octan-3-yl)-6-fluoro-2-(((2R,7aS)-2-fluorotetrahydro-1H-pyrrolizin-7a(5H)-yl)methoxy)pyrido[2,3-d]pyrimidin-7-yl)-5-ethynyl-6-fluoronaphthalen-2-ol

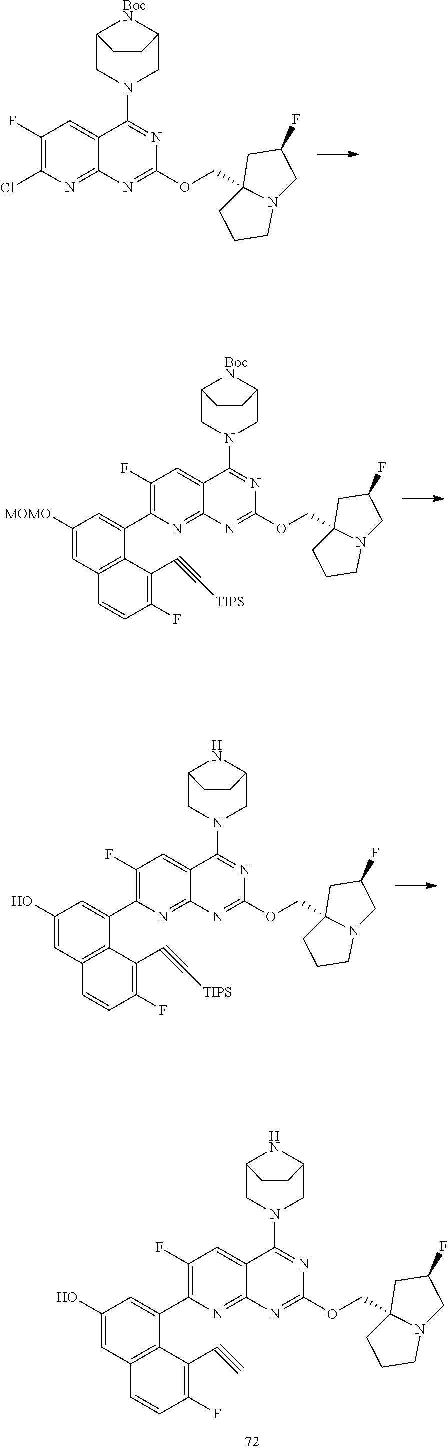

Step 1: tert-butyl (1R,5S)-3-(6-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[2,3-d]pyrimidin-4-yl)-3,8-diazabicyclo[3.2.1 ]octane-8-carboxylate

tert-butyl (1R,5S)-3-(7-chloro-6-fluoro-2-(((2R,7aS)-2-fluorotetrahydro- 1H-pyrrolizin-7a(5H)-yl)methoxy)pyrido[2,3-d]pyrimidin-4-yl)-3,8-diazabicyclo[3.2.1]octane-8-carboxylate (110 mg, 0.2 mmol), 3-methoxymethoxy-7-fluoro-8-(2-Triisopropylsilyl)ethynylnaphthalene-1-boronic acid pinacol ester (102 mg, 0.2 mmol), tris(dibenzylideneacetone)dipalladium (17 mg, 0.018 mmol), cesium carbonate, After mixing 1,4-dioxane (4 mL) and water (1 mL), the mixture was heated to 120° C. under reflux, and the reaction was stirred for 16 hours. The reaction was cooled to room temperature and stirred overnight to give a pale yellow precipitate. The reaction mixture was diluted with water (2 mL) and the solids were collected by filtration. The crude product was slurried with methanol (10 mL) to give tert-butyl (1R,5S)-3-(6-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[2,3-d]pyrimidin-4-yl)-3,8-diazabicyclo[3.2.1]octane-8-carboxylate (157 mg, 87%) was carried to the next reaction without further purification. LC/MS(ESI): m/z=902[M+H]+.

### Step 2: 4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)pyrido[2,3-d]pyrimidin-7-yl)-6-fluoro-5-((triisopropylsilyl)ethynyl)naphthalen-2-ol

tert-butyl (1R,5S)-3-(6-fluoro-7-(7-fluoro-3-(methoxymethoxy)-8-((triisopropylsilyl)cthynyl)naphthalen-1-yl)-2-(((2R,7aS)-2-fluorotetrahydro-1H-pyrrolizin-7a(5H)-yl)methoxy)pyrido[2,3-d]pyrimidin-4-yl)-3,8-diazabicyclo[3.2.1]octane-8-carboxylate (153 mg, 0.17 mmol) in 1 ml of ethyl acetate and 1 N HCl in 2 ml of 1,4-dioxane. After stirring at room temperature for 2 hours. the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The 4-(4-((1R,5S)-3,8-diazabicyclo[3.2.1 ]octan-3-yl)-6-fluoro-2-(((2R,7aS)-2-fluorotetrahydro-1H-pyrrolizin-7a(5H)-yl)methoxy)pyrido[2,3-d]pyrimidin-7-yl)-6-fluoro-5-((triisopropylsilyl)ethynyl)naphthalen-2-ol was obtained (99 mg, 77% yield). It was used directly in the next step. LC/MS (ESI): m/z=758 [M+H]+.

### Step 3: 4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)pyrido[2,3-d]pyrimidin-7-yl)-5-ethynyl-6-fluoronaphthalen-2-ol

Under nitrogen protection, 4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)pyrido[2,3-d]pyrimidin-7-yl)-6-fluoro-5-((triisopropylsilyl)ethynyl)naphthalen-2-ol (98 mg, 0.13 mmol) was dissolved in DMF (1 ML), CsF was added, then stirred at room temperature for 2 hours, and then purified by column chromatography to give compound 4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)pyrido[2,3-d]pyrimidin-7-yl)-5-ethynyl-6-fluoronaphthalen-2-ol (22 mg, 28% yield) as a yellow solid. LC/MS (ESI): m/z=601.2 [M+H]+.

### Example 59

### 6-Fluoro-7-(3-hydroxy-7-fluoro-8-ethylnaphthyl)-4-(3,6-diazabicyclo[3.1.1]heptan-6-yl)-2-((Preparation of (2R,7aS)-2-fluorotetrahydro-1H-pyrinyl-7a(5H)-yl)methoxy)pyridine[2,3-d]lopyrimidine

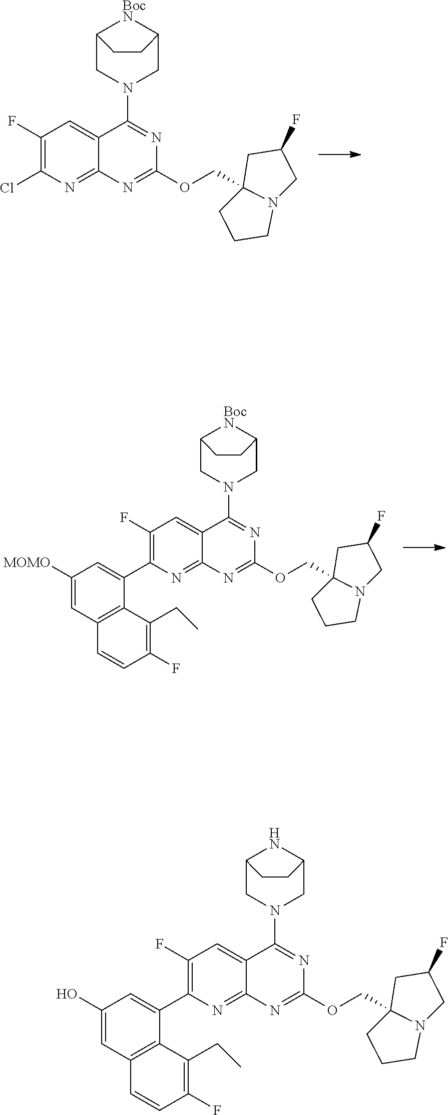

### Step 1: tert-butyl (1R,5S)-3-(7-(8-ethyl-7-fluoro-3-(methoxymethoxy)naphthalen-1-yl)-6-fluoro-2-(((2R,7aS)-2-fluorotetrahydro-1H-pyrrolizin-7a(5H)-yl)methoxy)pyrido[2,3-d]pyrimidin-4-yl)-3,8-diazabicyclo[3.2.1]octane-8-carboxylate

tert-butyl (1R,5S)-3-(7-chloro-6-fluoro-2-(((2R,7aS)-2-fluorotetrahydro-1H-pyrrolizin-7a(5H)-yl)methoxy)pyrido[2,3-d]pyrimidin-4-yl)-3,8-diazabicyclo[3.2.1]octane-8-carboxylate (110 mg, 0.2 mmol), 3-methoxymethoxy-7-fluoro-8 - Ethylnaphthalene-1-boronic acid pinacol ester (72.4 mg, 0.2 mmol). tris(dibenzylideneacetone)dipalladium (17 mg, 0.018 mmol), cesium carbonate. 1,4-dioxane (4 mL) and water (1 mL) were mixed, then heated to 120° C. under reflux, and the reaction was stirred for 16 hours. The reaction was cooled to room temperature and stirred overnight to give a pale yellow precipitate. The reaction mixture was diluted with water (2 mL) and the solid was collected by filtration. The crude product was slurried with methanol (10 mL) to give a yellow solid tert-butyl (1R,5S)-3-(7-(8-ethyl-7-fluoro-3-(methoxymethoxy)naphthalen-1-yl)-6-fluoro-2-(((2R,7aS)-2-fluorotetrahydro-1H-pyrrolizin-7a(5H)-yl)methoxy)pyrido[2,3-d]pyrimidin-4-yl)-3,8-diazabicyclo[3.2.1 ]octane-8-carboxylate (123 mg, 82%), proceeded to the next reaction without further purification. LC/MS(ESI): m/z=750[M+H]+.

### Step 2: 4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)pyrido[2,3-d]pyrimidin-7-yl)-5-ethyl-6-fluoronaphthalen-2-ol

tert-butyl (1R,5S)-3-(7-(8-ethyl-7-fluoro-3-(methoxymethoxy)naphthalen-1-yl)-6-fluoro-2-(((2R,7aS)-2-fluorotetrahydro-1H-pyrrolizin-7a(5H)-yl)methoxy)pyrido[2.3-d]pyrimidin-4-yl)-3.8-diazabicyclo[3.2.1 ]octane-8-carboxylate (127 mg, 0.17 mmol) in 1 ml of ethyl acetate and 1 N HCl in 2 ml of 1,4-dioxane. After stirring at room temperature for 2 hours, the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 4-(4-((1R,5S)-3,8-diazabicyclo[3.2.1]octan-3-yl)-6-fluoro-2-(((2R,7aS)-2-fluorotetrahydro-1H-pyrrolizin-7a(5H)-yl)methoxy)pyrido[2,3-d]pyrimidin-7-yl)-5-ethyl-6-fluoronaphthalen-2-ol (77 mg, 75% yield). LC/MS (ESI): m/z=605.2 [M+H]+.

### Example 60

### 4-(8-((1R,5S)-3,8-diazabicyclo[3.2.1]octan-3-yl)-6-(((2R,7aS)-2-fluorotetrahydro-1H-pyrrolizin-7a(5H)-yl)methoxy)-4-methylpyrimido[5,4-c]pyridazin-3-yl)-5-ethynyl-6-fluoronaphthalen-2-ol (Compound 60)

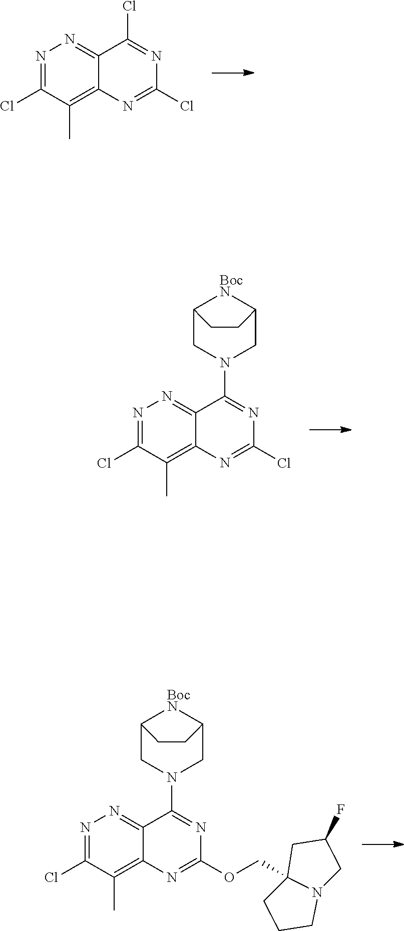 [SMILES: Cc1c(Cl)nnc2c(Cl)nc(Cl)nc12 ; Cc1c(Cl)nnc2c(N3CC4CCC(C3)N4C(=O)OC(C)(C)C)nc(Cl)nc12 ; Cc1c(Cl)nnc2c(N3CC4CCC(C3)N4C(=O)OC(C)(C)C)nc(OC[C@@]34CCCN3C[C@H](F)C4)nc12]

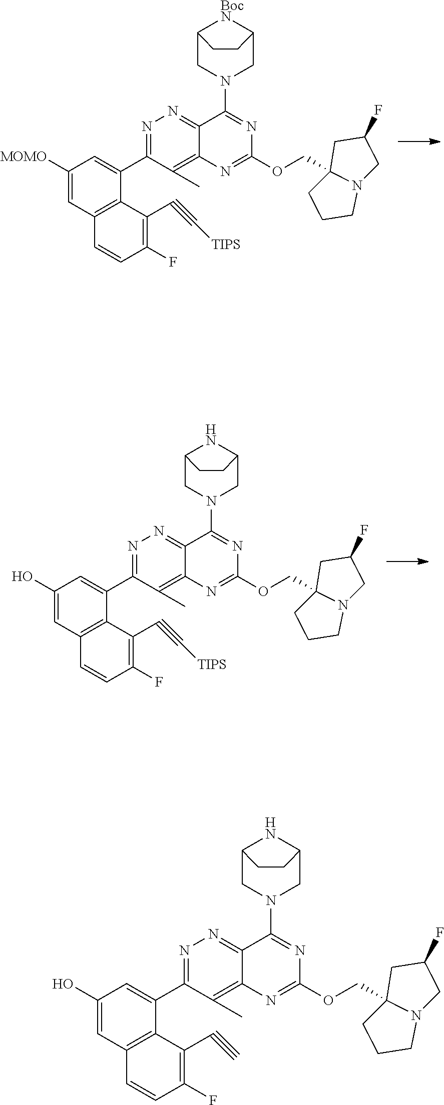

Compound 60 (70 mg, 72% yield) was obtained refer to preparations in Example 53 and Example 58. LC/MS (ESI): m/z=578 [M+H]+.

### Example 61

### 4-(8-((1R,5S)-3,8-diazabicyclo[3.2.1]octan-3-yl)-4-fluoro-6-(((2R,7aS)-2-fluorotetrahydro-1H-pyrrolizin-7a(5H)-yl)methoxy)pyrimido[5,4-c]pyridazin-3-yl)-5-ethynyl-6-fluoronaphthalen-2-ol (Compound 61)

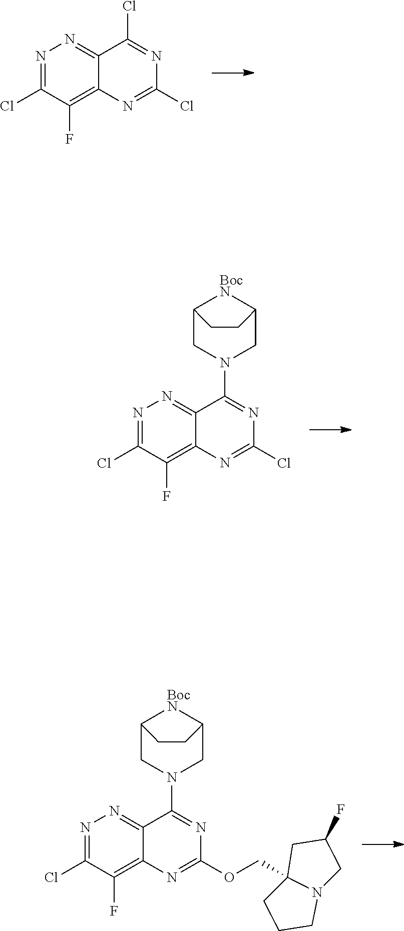 [SMILES: Fc1c(Cl)nnc2c(Cl)nc(Cl)nc12 ; CC(C)(C)OC(=O)N1C2CCC1CN(c1nc(Cl)nc3c(F)c(Cl)nnc13)C2 ; CC(C)(C)OC(=O)N1C2CCC1CN(c1nc(OC[C@@]34CCCN3C[C@H](F)C4)nc3c(F)c(Cl)nnc13)C2]

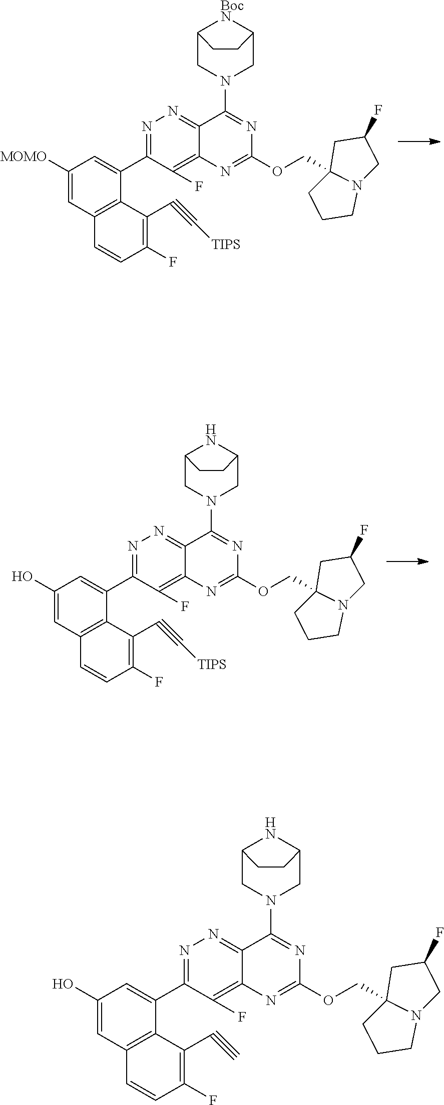

Compound 61 (80 mg, 78% yield) was obtained referred to preparations in Example 60. LC/MS (EST): m/z=602 [M+H]+.

Similar to the synthetic route of 1-60, the following compounds can be obtained:

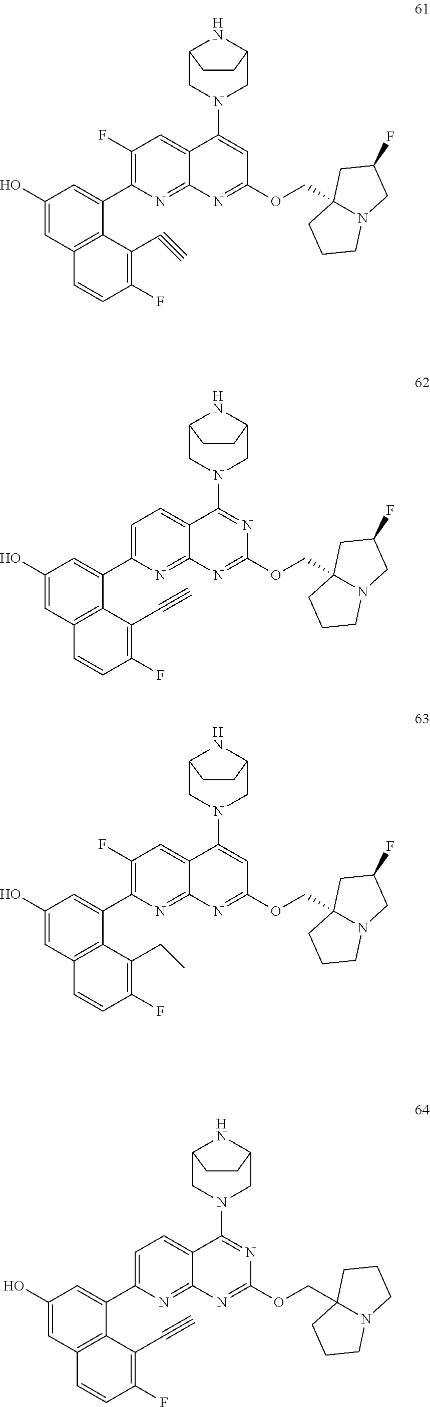 [SMILES: C#Cc1c(F)ccc2cc(O)cc(-c3nc4nc(OC[C@@]56CCCN5C[C@H](F)C6)cc(N5CC6CCC(C5)N6)c4cc3F)c12 ; C#Cc1c(F)ccc2cc(O)cc(-c3ccc4c(N5CC6CCC(C5)N6)nc(OC[C@@]56CCCN5C[C@H](F)C6)nc4n3)c12 ; CCc1c(F)ccc2cc(O)cc(-c3nc4nc(OC[C@@]56CCCN5C[C@H](F)C6)cc(N5CC6CCC(C5)N6)c4cc3F)c12 ; C#Cc1c(F)ccc2cc(O)cc(-c3ccc4c(N5CC6CCC(C5)N6)nc(OCC56CCCN5CCC6)nc4n3)c12]

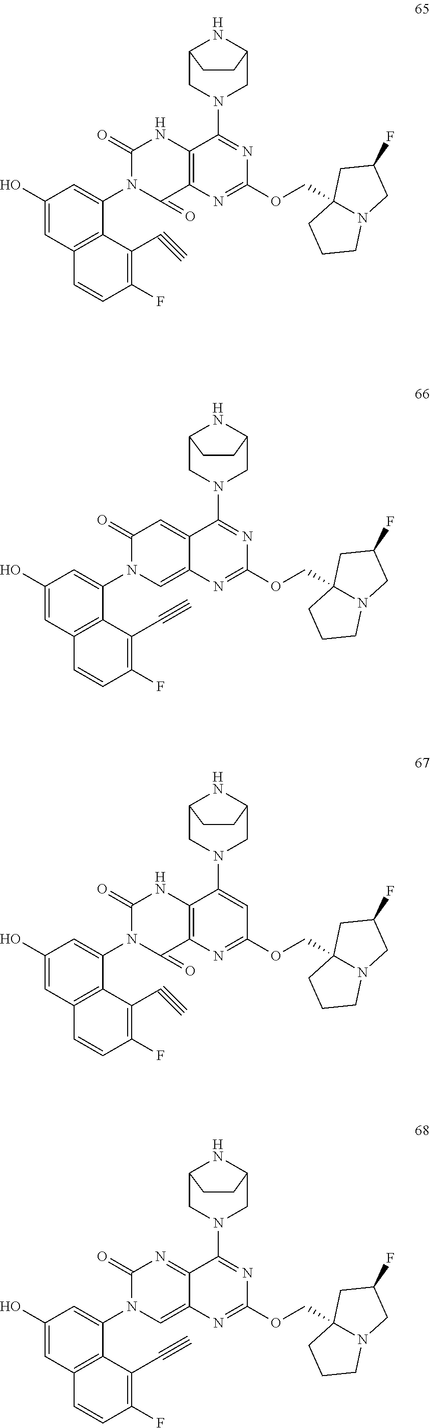 [SMILES: C#Cc1c(F)ccc2cc(O)cc(-n3c(=O)[nH]c4c(N5CC6CCC(C5)N6)nc(OC[C@@]56CCCN5C[C@H](F)C6)nc4c3=O)c12 ; C#Cc1c(F)ccc2cc(O)cc(-n3cc4nc(OC[C@@]56CCCN5C[C@H](F)C6)nc(N5CC6CCC(C5)N6)c4cc3=O)c12 ; C#Cc1c(F)ccc2cc(O)cc(-n3c(=O)[nH]c4c(N5CC6CCC(C5)N6)cc(OC[C@@]56CCCN5C[C@H](F)C6)nc4c3=O)c12 ; C#Cc1c(F)ccc2cc(O)cc(-n3cc4nc(OC[C@@]56CCCN5C[C@H](F)C6)nc(N5CC6CCC(C5)N6)c4nc3=O)c12]

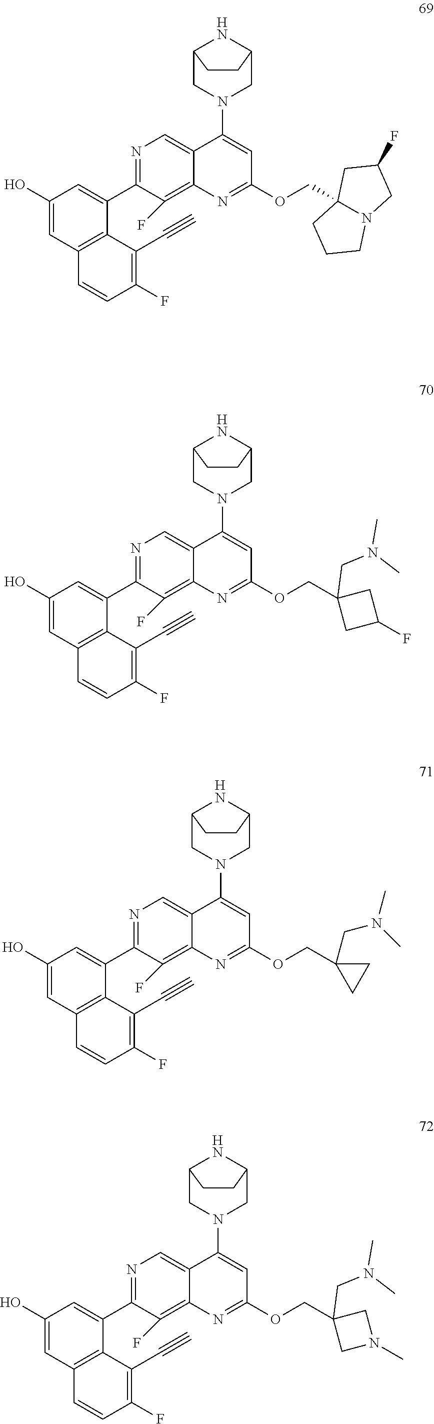 [SMILES: C#Cc1c(F)ccc2cc(O)cc(-c3ncc4c(N5CC6CCC(C5)N6)cc(OC[C@@]56CCCN5C[C@H](F)C6)nc4c3F)c12 ; C#Cc1c(F)ccc2cc(O)cc(-c3ncc4c(N5CC6CCC(C5)N6)cc(OCC5(CN(C)C)CC(F)C5)nc4c3F)c12 ; C#Cc1c(F)ccc2cc(O)cc(-c3ncc4c(N5CC6CCC(C5)N6)cc(OCC5(CN(C)C)CC5)nc4c3F)c12 ; C#Cc1c(F)ccc2cc(O)cc(-c3ncc4c(N5CC6CCC(C5)N6)cc(OCC5(CN(C)C)CN(C)C5)nc4c3F)c12]

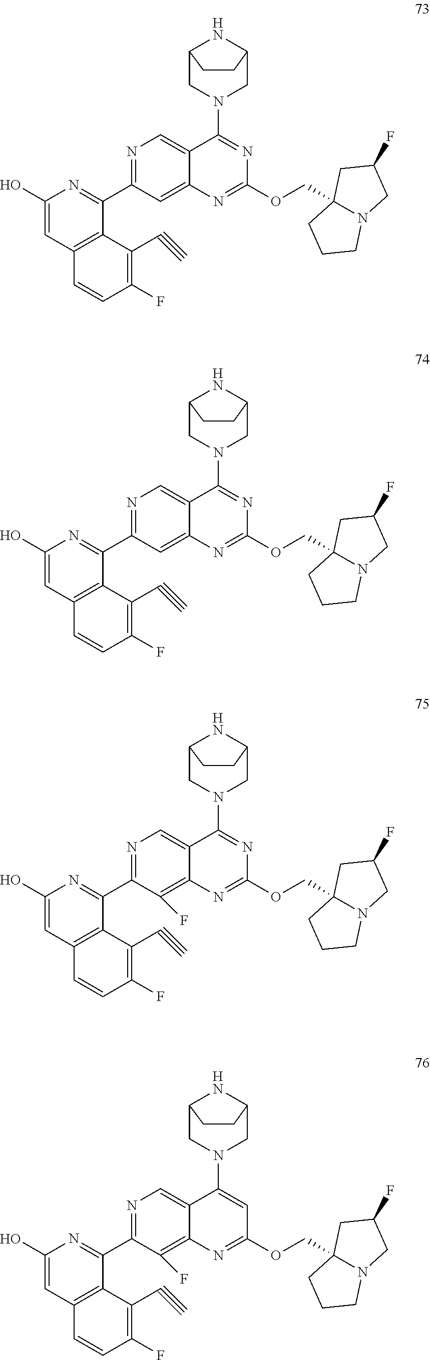 [SMILES: C#Cc1c(F)ccc2cc(O)nc(-c3cc4nc(OC[C@@]56CCCN5C[C@H](F)C6)nc(N5CC6CCC(C5)N6)c4cn3)c12 ; C#Cc1c(F)ccc2cc(O)nc(-c3cc4nc(OC[C@@]56CCCN5C[C@H](F)C6)nc(N5CC6CCC(C5)N6)c4cn3)c12 ; C#Cc1c(F)ccc2cc(O)nc(-c3ncc4c(N5CC6CCC(C5)N6)nc(OC[C@@]56CCCN5C[C@H](F)C6)nc4c3F)c12 ; C#Cc1c(F)ccc2cc(O)nc(-c3ncc4c(N5CC6CCC(C5)N6)cc(OC[C@@]56CCCN5C[C@H](F)C6)nc4c3F)c12]

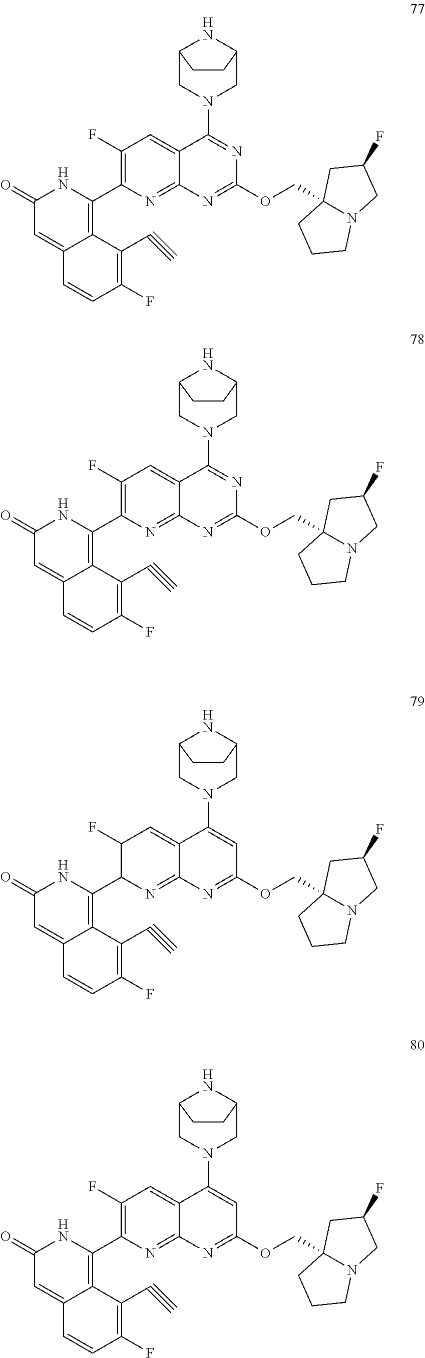 [SMILES: C#Cc1c(F)ccc2cc(=O)[nH]c(-c3nc4nc(OC[C@@]56CCCN5C[C@H](F)C6)nc(N5CC6CCC(C5)N6)c4cc3F)c12 ; C#Cc1c(F)ccc2cc(=O)[nH]c(-c3nc4nc(OC[C@@]56CCCN5C[C@H](F)C6)nc(N5CC6CCC(C5)N6)c4cc3F)c12 ; C#Cc1c(F)ccc2cc(=O)[nH]c(C3N=c4nc(OC[C@@]56CCCN5C[C@H](F)C6)cc(N5CC6CCC(C5)N6)c4=CC3F)c12 ; C#Cc1c(F)ccc2cc(=O)[nH]c(-c3nc4nc(OC[C@@]56CCCN5C[C@H](F)C6)cc(N5CC6CCC(C5)N6)c4cc3F)c12]

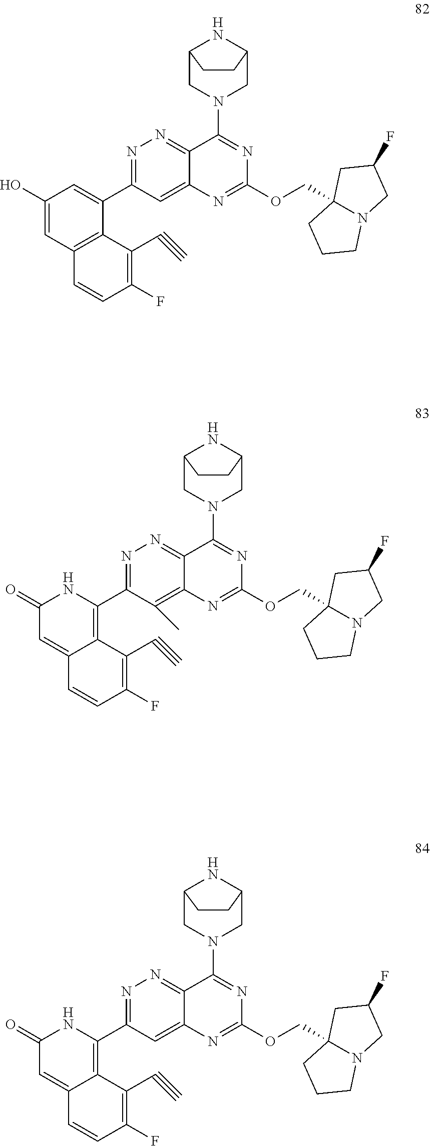 [SMILES: C#Cc1c(F)ccc2cc(O)cc(-c3cc4nc(OC[C@@]56CCCN5C[C@H](F)C6)nc(N5CC6CCC(C5)N6)c4nn3)c12 ; C#Cc1c(F)ccc2cc(=O)[nH]c(-c3nnc4c(N5CC6CCC(C5)N6)nc(OC[C@@]56CCCN5C[C@H](F)C6)nc4c3C)c12 ; C#Cc1c(F)ccc2cc(=O)[nH]c(-c3cc4nc(OC[C@@]56CCCN5C[C@H](F)C6)nc(N5CC6CCC(C5)N6)c4nn3)c12]

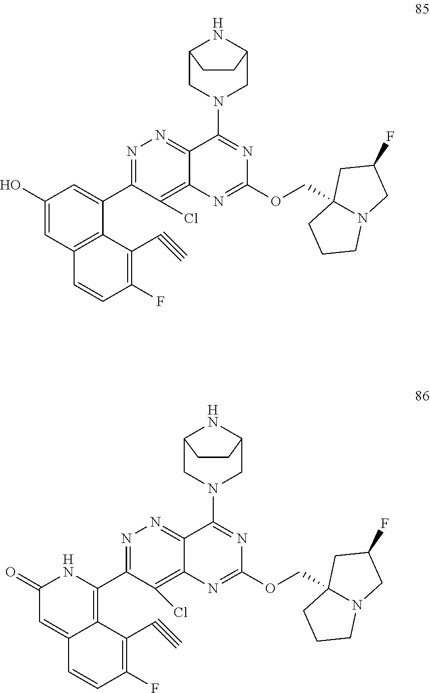 [SMILES: C#Cc1c(F)ccc2cc(O)cc(-c3nnc4c(N5CC6CCC(C5)N6)nc(OC[C@@]56CCCN5C[C@H](F)C6)nc4c3Cl)c12 ; C#Cc1c(F)ccc2cc(=O)[nH]c(-c3nnc4c(N5CC6CCC(C5)N6)nc(OC[C@@]56CCCN5C[C@H](F)C6)nc4c3Cl)c12]

### Example 62 Biological Activity Test

The present invention is further described and explained below in conjunction with test examples, but these implementations are not meant to limit the scope of the present invention.

### 1. KRAS-G2D/SOS1 Binding Experiment

### 1 Experimental Method

A) Dilute Tagged-His Tag G12D protein and Tag1-SOS protein with diluent at 1:100, and use detection buffer for anti-Tag1-Tb3+ antibody and anti-Tag2-XL665 at 1:100 or 1:25, respectively Dilute.
B) Dilute the compound to be tested with diluent starting from 10000 mM concentration, 4 times gradient, a total of 6 concentration gradients, and dilute to 10× stock solution.
C) In a 96-well plate, add 4 uL Tagged-His Tag G12D protein, 4 uL Tag1-SOS protein, 2 ul dilution solution (positive control) or test compound (different concentrations of 10× stock solution) to each well in sequence, a total of 10 ul, and incubate at room temperature for 15 10 minutes later, add pre-mixed 5 uL anti-Tag1-Tb3+ and 5 uL anti-Tag2-xL665, seal the plate, incubate at room temperature for 2 hours, and read the HTRF signal with a TECAN INFINITEF NANO+ microplate reader.

### 2 Experimental Results

The ratio of love body and donor excitation signals for each single well was calculated using the formula ratio=665 nm signal value/620 nm signal value×104. Data were processed using Graphpadprism5 software. IC₅₀ values were calculated by sigmoidal dose-response curve fitting. “+” means IC₅₀≤50 nM; “++” means 50<IC₅₀≤500 nM; “+++” means 500 nM<IC₅₀, the results are shown in Table 1 below

TABLE 1
IC₅₀(nm) of compounds inhibiting
KRAS-G2D/SOS1 binding activity

| | KRAS-G2D/SOS1 binding | | KRAS-G2D/SOS1 binding |
|---|---|---|---|
| NO | IC₅₀(nm) | No | IC₅₀(nm) |
| 1 | +++ | 45 | + |
| 7 | +++ | 46 | + |
| 33 | ++ | 54 | + |
| 34 | ++ | 57 | + |
| 36 | + | 58 | + |

### 2. Tumor Cell Proliferation Inhibition Experiment

### 1 Experimental Method

The ATCC CRL-1739 (KRASG¹²D mutant) cells were digested, centrifuged and resuspended, and the cell density was measured with a Scepter automatic cell counter. The cells were diluted to a solution containing 44,000 cells per milliliter, and the density-adjusted cell solution was 90 per well. Microliters were added to a 96-well culture plate. The 96-well plate was placed in a 37° C., 5% CO₂ incubator. After the cells were cultured for 24 hours, different concentrations of the compounds to be tested were added. The cells were incubated with the compounds for 72 hours in the presence of 10% fetal bovine serum. Glo Luminescent Cell Viability Assay Kit (see manufacturer's instructions for details) was used to measure ATP content to assess cell growth inhibition. Briefly, 30 μl of Cell Titer-Glo reagent was added to each well, and the plate was shaken for 10 minutes to induce cell lysis. FluoroskanAscentFL (Thermo) assay records the fluorescence signal, and the maximum signal value is obtained from cells treated with DMSO for 72 hours. The minimum signal value was obtained from the medium alone (the number of cells was zero), and the % inhibition rate=(the maximum signal value compound signal value)/(the maximum signal value−the minimum signal value×100%, and the data were processed using Graphpadprism 5 software. By sigmoid Dose-response curve fitting to calculate IC50 value. “A” means IC50≤50 nM; “B” means 50<IC50≤500 nM; “C” means 500<IC50≤2000 nM; “D” means 2000<IC50

### 20 Experimental Results

Calculate the IC₅₀ of each compound in the above experiment, and the results are shown in Table 2 below

TABLE 2
IC₅₀(nm) of inhibitory activity of compounds
on tumor cell proliferation.

| Example code | CRL-1739 IC₅₀(nm) |
|---|---|
| 1 | D |
| 2 | D |
| 7 | D |
| 32 | D |
| 33 | B |
| 34 | C |
| 35 | D |
| 36 | D |
| 37 | D |
| 38 | D |
| 39 | D |
| 40 | D |
| 41 | D |
| 42 | D |
| 43 | C |
| 44 | C |
| 45 | B |
| 46 | A |
| 47 | B |
| 48 | A |
| 49 | C |
| 50 | C |
| 51 | B |
| 52 | B |
| 53 | A |
| 54 | A |
| 55 | A |
| 56 | A |
| 57 | A |
| 58 | A |
| 59 | A |

Although the present invention has been described in detail above, it will be understood by those skilled in the art that various modifications and variations can be made in the present invention without departing from the spirit and scope of the invention. The scope of the rights of the present invention is not limited to the detailed description above, but should be attributed to the claims.

1. A compound of formula (I): 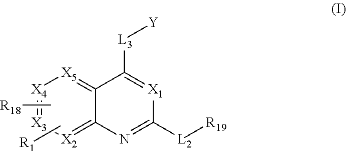
Wherein,
Each L₁ at each occurrence is independently selected from bond, —C₁₋₄alkyl-, —CR₈R₉—, —C₁₋₂alkyl(R₈)(OH)—, —C(O)—, —CR₈R₉O—, —OCR₈R₉—, —SCR₈R₉—, —CR₈R₉S—, —NR₈—, —NR₈C(O)—, —C(O)NR₈—, —NR₈C(O)NR₉—, —CF₂—, —O—, —S—, —S(O)ₘ—, —NR₈S(O)ₘ—, —S(O)ₘNR₈—;
Each R₁ at each occurrence is independently selected from phenyl, naphthyl, 5-membered heteroaryl, 6-membered heteroaryl, 7-membered heteroaryl, 8-membered heteroaryl, 9-membered heteroaryl, or 10-membered heteroaryl heteroaryl, each heteroaryl independently at each occurrence containing 1, 2, 3 or 4 heteroatoms selected from N, O, or S; each R1 at each occurrence independently and optionally substituted or unsubstituted with 1, 2, 3, 4, 5 or 6 R₂₀;
Each R₂₀ at each occurrence is independently selected from deuterium, halogen, oxo, —C₁₋₆ alkyl, —C₂₋₆ alkenyl, —C₂-alkynyl, —C₁₋₆ alkylene-(halogen)₁₋₃, C₁₋₆ heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene-(halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR6C(═O)R₇, —S(O)₂NR₆R₇ or —C₃₋₆ carbocyclyl; each R₂₀ is independently and optionally substituted or unsubstituted with 1, 2, 3, 4, 5 or 6 substituents selected from deuterium, halogen, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇.
Each X₁, X₂, X₃, X₄, X₅ is independently selected from N, CR₂₁ at each occurrence;
Each R₂₁ is independently selected from H, D, cyano, halogen, C₁₋₆ alkyl, COOH, NHCOH, CONH₂, OH or —NH₂;
Each R₁₈ is independently selected from H, D, cyano, halogen, C₁₋₆ alkyl, COOH, NHCOH, CONH₂, OH or —NH₂;
Each L₂ is independently selected at each occurrence from bond, OC₀₋₆ alkyl, NC₀₋₆ alkyl, C₁₋₆ alkyl, COC₀₋₆ alkyl or SC₀₋₆ alkyl;
Each R₁₉ is independently selected from 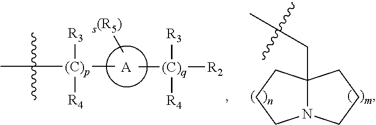
—C₁₋₆ alkyl, —C₂₋₆ alkenyl, —C₂-alkynyl, —C₁₋₆ alkylene-(halogen)₁₋₃, C₁₋₆ heteroalkyl , —CN, —OR₆, —C₁₋₆ alkylene-(OR₆)₁₋₃, —O—C₁₋₆ alkylene-(halogen)₁₋₃, —SR₆, —S—C₁₋₆ alkylene Base-(halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR₆C(═O)R₇, —S(O)₂NR₆R₇, —C₃₋₆ carbocyclyl, 3-8 membered heterocycle; 3-8 membered heterocycle independently at each occurrence contains 1, 2, 3 or 4 heteroatoms selected from N, O, or S; each R₁₉ is independently and optionally are substituted or unsubstituted with 1, 2, 3, 4, 5 or 6 substituents selected from deuterium, halogen, —C₁₋₆alkane base, —C₁₋₆alkoxy, oxo, —OR₆, —NR₆R₇, —CN, —C(═O)R₆, —C(═O)OR₆, —OC(═O)R₆, —C(═O) Substituents of NR₆R₇, —NR₆C(═O)R₇ or —S(O)₂NR₆R₇.
Each ring A is a C₃₋₁₀ carbocyclic ring, and the 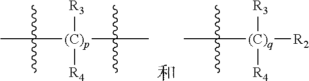
can be attached to the same carbon atom or to a different atom of the ring A;
Each R₂ is —OR₆, —NR₆R₇, —SR₆, —S(═O)R₆, —S(═O)₂R₆, 5-10 membered heteroaryl or 3-10 membered heterocyclyl, each heterocyclyl and heteroaryl at each occurrence independently contain 1, 2, 3 or 4 heteroatoms selected from N, O, S, S═O or S(═O)₂, each R₂ at each occurrence independently and optionally substituted or unsubstituted with 1, 2, 3, 4, 5 or 6 R₂₂;
Each R₃ and R₄ at each occurrence is independently selected from deuterium, hydrogen, halogen, —C₁₋₆ alkyl, —C₂₋₆ alkenyl, —C₂₋₆ alkynyl, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇ or —C₃₋₁₀ carbocyclyl, each heterocyclyl and heteroaryl at each occurrence independently contain 1, 2, 3 or 4 heteroatonsselected from N, O, S, S═O or S(═O)₂; each R₃ and R₄ at each occurrence is independently optionally substituted or unsubstituted with 1, 2, 3, 4, 5 or 6 substituents selected from deuterium, halogen, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇;
Each R₅ at each occurrence is independently selected from deuterium, halogen, oxo, —C₁₋₆ alkyl, —C₁₋₆ alkylene-(halogen)₁₋₃, C₁₋₆ heteroalkyl, —CN , —OR₆, —C₁₋₆ alkylene-(OR₆)₁₋₃, —O—C₁₋₆ alkylene-(halogen)₁₋₃, —SR₆, —S—C₁₋₆ alkylene-(Halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl, each heterocyclyl and heteroaryl independently at each occurrence contains 1, 2, 3 or 4 options A heteroatom from N, O, S, S═O or S(═O)₂; each R₃ and R₄ at each occurrence independently and optionally substituted or unsubstituted with 1, 2, 3, 4, 5 or 6 substituents selected from deuterium, halogen, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇;
Each R₆ and R₇ at each occurrence is independently selected from hydrogen or —C₁₋₆ alkyl, each R₆ and R₇ is independently optionally substituted with 1, 2, 3, 4, 5 or 6 R₂₂ or not or R₇ and R₇ together with the N atom to which they are connected together form a 3-10-membered heterocycle, and the 3-10-membered heterocycle may further comprise 1, 2, 3 or 4 selected from N, O, S, A heteroatom of S(═O) or S(═O)₂, and the 3-10 membered heterocycle is independently optionally substituted or unsubstituted by 1, 2, 3, 4, 5 or 6 R₂₂;
Each R₂₂ at each occurrence is independently selected from deuterium, halogen, oxo, —C₁₋₆alkyl, —C₁₋₆alkylene-(halogen)₁₋₃, C₁₋₆heteroalkyl, —CN , —O—C₁₋₆ alkyl, —C₁₋₆ alkylene-(O—C₁₋₆ alkyl₁₋₃, —O—C₁₋₆ alkylene-(halogen)₁₋₃, —S—C₁₋₆ alkyl, —S—C₁₋₆ alkylene-(halogen)₁₋₃, —N—C₁₋₆ alkyl—C₁₋₆ alkyl, —C₁₋₆ alkylene-NC₁₋₆ alkyl base C₁₋₆ alkyl, —C(═O)C₁₋₆ alkyl, —C(═O)OC₁₋₆ alkyl, —OC(═O)C₁₋₆ alkyl, —C(═O)NC₁₋₆ alkyl C₁₋₆ alkyl, —NC₁₋₆ alkyl C(═O)C₁₋₆ alkyl, —S(O)₂NC₁₋₆ alkyl C₁₋₆ alkyl or —C₃₋₆ carbocyclyl;
s is selected from 0, 1, 2, 3, 4, 5 or 6;
p is selected from 0, 1, 2, 3, 4, 5 or 6;
q is selected from 0, 1, 2, 3, 4, 5 or 6;
m is selected from 1, 2, 3;
n is selected from 1, 2, 3
Y is absent or selected from 3-8 membered cycloalkyl, 3-8 membered heterocycloalkyl, 5-12 membered fused alkyl, 5-12 membered fused heterocyclyl, 5-12 membered spirocyclyl, 5-12 membered Membered spiroheterocyclyl, aryl or heteroaryl, each heterocycloalkyl, fused heterocyclyl, spiroheterocyclyl, heteroaryl at each occurrence independently contains 1, 2, 3 or 4 heteroatoms selected from N, O, or S, wherein the cycloalkyl, heterocycloalkyl, spiro, fused ring, fused heterocyclyl, spiroheterocyclyl, aryl, or heteroaryl group is optionally replaced by one or more G¹s;
G¹ and G² are each independently selected from deuterium, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR₈, —OC(O)NR₈R₉, —C(O)OR₈, —C(O)NR₈R₉, —C(O)R₈, —NR₈R₉, —NR₈C(O)R₉, —NR₈C(O)NR₉R₁₀, —S(O)ᵢR₈ or —NR₈S(O)ᵢR₉, wherein the alkyl, alkenyl, alkynyl, cycloalkyl, heterocycloalkyl, aryl, heteroaryl is optionally substituent with 1 or more deuterium, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀-aryl, 5-10-membered heteroaryl, —OR₁₁, —OC(O)NR₁₁R₁₂, —C(O)OR₁₁, —C(O)NR₁₁R₁₂, —C(O)R₁₁, —NR₁₁R₁₂, —NR1₁C(O)R₁₂, —NR₁₁C(O)NR₁₂R₁₃, —S(O)ᵢR₁₁ or —NR₁₁S(O)ᵢR₁₂;
R₈, R₉, R₁₀, R₁₁, R₁₂ and R₁₃ are each independently selected from hydrogen, deuterium, cyano, halogen, C₁₋₆ alkyl, C₃₋₈ cycloalkyl or 3-8 membered monocyclic heterocyclyl, monocyclic heterocyclyl aryl or phenyl;
and i is 1 or 2.

2. The compound of formula (I), a pharmaceutically acceptable salt thereof, or a stereoisomer thereof according to claim 1, wherein each R₁ at each occurrence is independently selected from phenyl, naphthyl, 5-membered heteroaryl, 6-membered heteroaryl, 7-membered heteroaryl, 8-membered heteroaryl, 9-membered Heteroaryl or 10-membered heteroaryl, each heteroaryl independently at each occurrence containing 1, 2, 3 or 4 heteroatoms selected from N, O, or S; each R₁ at each occurrence is independently and optionally substituted or unsubstituted with 1, 2, 3, 4, 5 or 6 R₂₀;
Preferably, each R₁ at each occurrence is independently selected from phenyl, naphthyl, pyridyl, indolyl, indazolyl, benzofuranyl, benzothienyl, quinolyl, iso Quinolinyl, each R₁ at each occurrence is independently and optionally substituted or unsubstituted with 1, 2, 3, 4, 5 or 6 R₁₂; each R₁ at each occurrence independently optionally substituted or unsubstituted with 1, 2, 3, 4, 5 or 6 R₂₀;
Preferably, each R₁ is selected from: 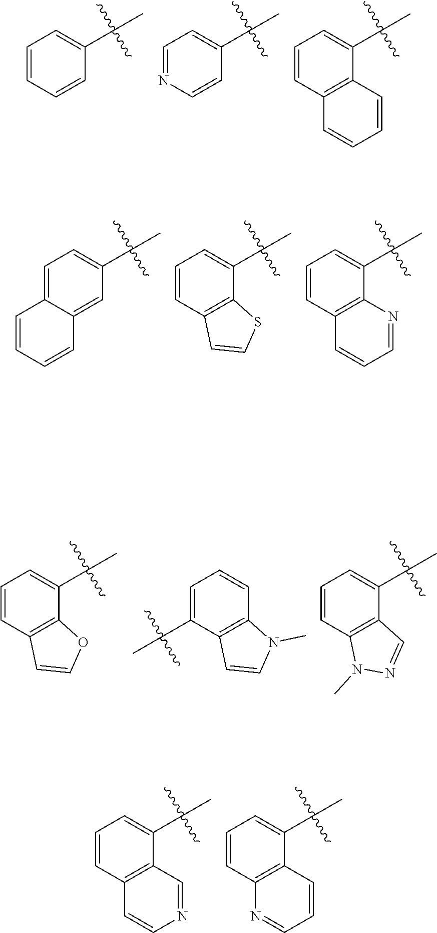
Preferably, each R₁ is independently and optionally substituted or unsubstituted at each occurrence with 1, 2, 3, 4, 5, or 6 R₂₀;
Preferably, each R₂₀ at each occurrence is independently selected from deuterium, halogen, oxo, —C₁₋₆alkyl, —C₁₋₆alkylene-(halogen)₁₋₃, C₁₋₆ Heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene-(halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl; each R₁₂ is independently and optionally substituted or unsubstituted with 1, 2, 3, 4, 5 or 6 substituents selected from deuterium, halogen, —C₁₋₆ alkyl, —C₁₋₆ alkoxy, oxo, —OR₆, —NR₆R₇, —CN, —C(═O)R₆, —C(═O)OR₆, Substituents of —OC(═O)R₆, —C(═O)NR₆R₇, —NR₆C(═O)R₇ or —S(O)₂NR₆R₇.;
Preferably, R₆ and R₇ in each R₂₀ are independently selected at each occurrence from hydrogen, deuterium, or —C₁₋₃ alkyl;
Preferably, each R₂₀ at each occurrence is independently selected from -deuterium, —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)2NH(CH₃), —S(O)₂N(CH₃)₂, 3-membered carbocyclyl, 4-membered carbocyclyl, 5-membered carbocyclyl or 6-membered carbocyclyl; each R₂₀ is independently and optionally substituted or unsubstituted with 1, 2, 3, 4, 5 or 6 substituents selected from -deuterium, —F, —Cl, —Br, methyl, ethyl, propyl , isopropyl, methoxy, ethoxy, propoxy, isopropoxy, oxo, _OH, —NH₂, —NHCH₃, _N(CH₃)₂, —CN, —C(═O)CH₃, _C(═O)OO, —OC(═O)O, —C(═O)NH₂, —C(═O)NH(CH₃), —C(═O)N(CH₃)₂, —NHC(═O)CH₃, —N(CH₃) of C(═O)CH₃, —S(O)₂NH₂, —S(O)₂NH(CH₃) or —S(O)₂N(CH₃)₂.
Preferably, each R₁ is selected from:

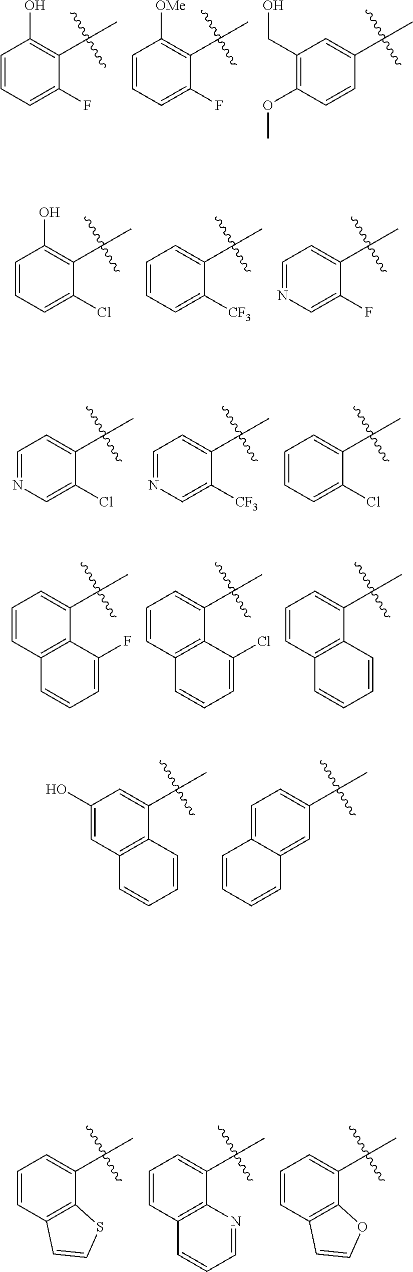

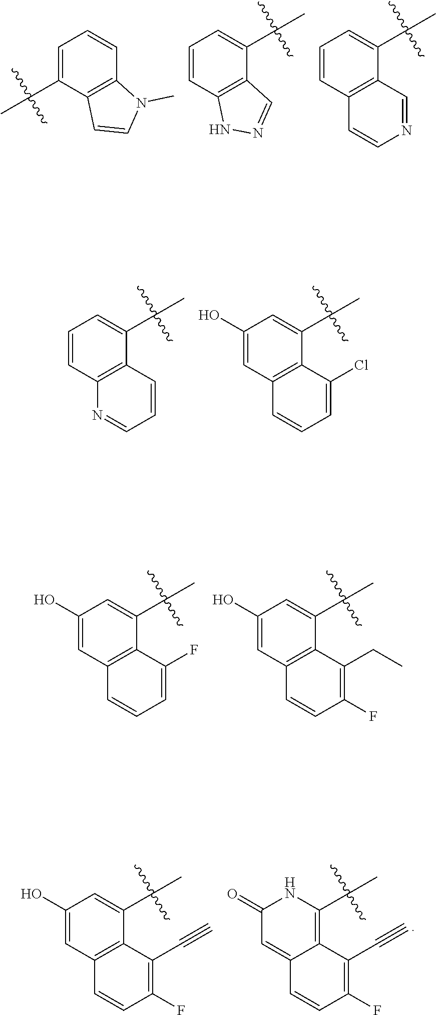

3. The compound of formula (I), a pharmaceutically acceptable salt thereof, or a stereoisomer thereof according to claim 1, wherein each ring A is absent, a 3-membered carbocycle, a 4-membered carbocycle, a 5-membered carbocycle, or a 6-membered carbocycle, and the 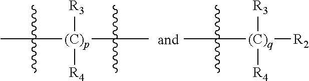
can be attached to the same ring A on a same carbon atom or on a different atom; each R₂ at each occurrence is independently selected from —NR₆R₇ or a 3-6 membered heterocyclyl, each heterocyclyl at each occurrence independently contains 1 heteroatom selected from N, each R₂ at each occurrence is independently and optionally substituted or unsubstituted with 1, 2, 3, 4, 5 or 6 R₂₀;
Preferably, R₆ and R₇ in each R₂ at each occurrence are independently selected from hydrogen, deuterium, methyl, ethyl, propyl, or isopropyl; or R₆ and R₇ in R₂ are taken together The connected N atoms together form a 3-6 membered heterocycle, the 3-6 membered heterocycle may further comprise 1 heteroatom selected from N, and the 3-6 membered heterocycle is independently and optionally substituted or unsubstituted with 1, 2, 3, 4, 5 or 6 R₂₀;
Preferably, each R₂ is at each occurrence independently selected from —NH₂, —N(CH₃)₂, —N(CH₃)(CH₂CH₃), —N(CH₂CH₃)₂, 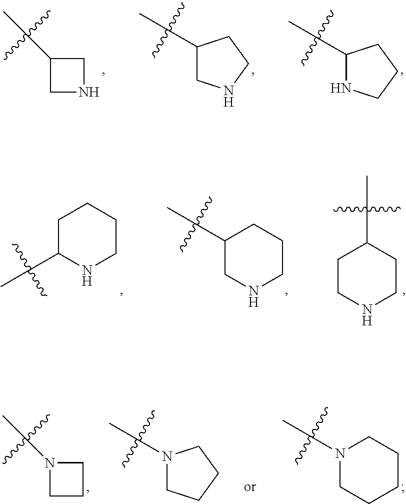
each R₂ is independently and optionally substituted or unsubstituted with 1, 2, 3, 4, 5 or 6 R₂₀;
Preferably, each R₂ at each occurrence is independently selected from —NH₂, —N(CH₃)₂, —N(CH₃)(CH₂CH₃), —N(CH₂CH₃)₂, 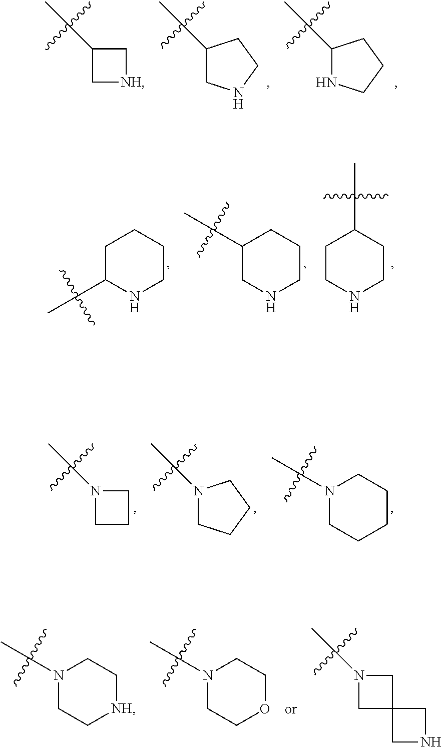
each R₂ is independently and optionally substituted or unsubstituted with 1, 2, 3, 4, 5 or 6 R₂₀;
Preferably, each R₂₀ at each occurrence is independently selected from deuterium, halogen, oxo, —C₁₋₆alkyl, —C₁₋₆alkylene-(halogen)₁₋₃, C₁₋₆ Heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene-(halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl; each R₁₂ is independently and optionally substituted or unsubstituted with 1, 2, 3, 4, 5 or 6 substituents selected from deuterium, halogen, —C₁₋₆ alkyl, —C₁₋₆ alkoxy, oxo, —OR₆, —NR₆R₇, —CN, —C(═O)R₆, —C(═O)OR₆, Substituents of —OC(═O)R₆, —C(═O)NR₆R₇, —NR₆C(═O)R₇ or —S(O)₂NR₆R₇
Preferably, each R₂₀ at each occurrence is independently selected from -deuterium, —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂. —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocyclyl, 5-membered carbocyclyl or 6-membered carbocyclyl; each R₂₀ is independently and optionally substituted or unsubstituted with 1, 2, 3, 4, 5 or 6 substituents selected from -deuterium, —F, —Cl, —Br, methyl, ethyl, propyl , isopropyl, methoxy, ethoxy, propoxy, isopropoxy, oxo, —OH, —NH₂, —NHCH₃, _N(CH₃)₂, —CN, —C(═O)CH₃, —C(═O)OO, —OC(═O)O, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

4. The compound of formula (I), a pharmaceutically acceptable salt thereof, or a stereoisomer thereof according to claim 1, wherein each R₃ and R₄ at each occurrence is independently selected from deuterium, hydrogen, halogen, —C₁₋₆ alkyl, —C₂₋₆ alkenyl, —C₂₋₆ alkynyl, oxo , —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇ or —C₃₋₁₀ carbocyclyl, each heterocyclyl and heteroaryl at each occurrence independently contain 1, 2, 3 or 4 heteroatoms selected from N, O, S, S═O or S(═O)₂; each R₃ and R₄ at each occurrence is independently and optionally substituted or unsubstituted with 1, 2, 3, 4, 5 or 6 atoms selected from deuterium, halogen, oxo, —C₁₋₆ alkyl, —C₁₋₆ alkoxy, oxo, —OR₆, —NRR, —CN, —C(═O)R, —C(═O)OR, —OC(═O)R, Substituents of —C(═O)NR₆R₇, —NR₆C(═O)R₇or —S(O)₂NR₆R₇;
Preferably, R₆ and R₇ in each of R₃ and R₄ at each occurrence are independently selected from hydrogen, deuterium, or —C₁₋₃ alkyl;
Preferably, each R₃ and R₄ at each occurrence is independently selected from hydrogen, —F, —Cl, —Br, methyl, ethyl, propyl, isopropyl, vinyl, propenyl, isopropenyl, ethynyl, propynyl, oxo, —OH, —OCH₃, —OCH₂CH₃, —OCH₂CH₂CH₃, —OCH(CH₃)₂, —NH₂, —NHCH₃, —NHCH₂CH₃, —NHCH₂CH₂CH₃, —NHCH(CH₃)₂. —N(CH₃)₂, —N(CH₃)CH₂CH₃, —N(CH₃)CH₂CH₂CH₃, —N(CH₃)CH(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocycle group, 5-membered carbocyclyl, or 6-membered carbocyclyl; each R₅ or R₆ is independently and optionally substituted or unsubstituted with 1, 2, 3, 4, 5 or 6 substituents selected from —F, —Cl, —Br, oxo , methyl, ethyl, propyl, isopropyl, —OH, OCH₃, —OCH₂CH₃, —OCH₂CH₂CH₃, —OCH(CH₃)₂, —NH₂, —N(CH₃)₂, —CN, —C(═O)CH₃, —OC(═O)CH₃, —C(═O)NH₂, —C(═O)NH(CH₃), —C(═O)N(CH₃)₂, —NHC(═O)CH₃, Substituents of —N(CH₃)C(═O)CH₃, —S(O)₂NH₂, —S(O)₂NH(CH₃), —S(O)₂N(CH₃)₂.

5. The compound of formula (I), a pharmaceutically acceptable salt thereof, or a stereoisomer thereof according to claim 1, wherein each R₅ at each occurrence is independently selected from deuterium, —F, —Cl, —Br, —C₁₋₃alkyl, —C₁₋₃alkylene-(halogen)₁₋₃. C₁₋₃ heteroalkyl, —C₂₋₃ alkenyl, —C₂₋₃ alkynyl, —CN, —OR₆, —C₁₋₆ alkylene-(OR₆)₁₋₃, —O—C₁₋₆ Alkylene-(halogen)₁₋₃, —SR₆, —S—C₁₋₆Alkylene-(halogen)₁₋₃, —NR₆R₇, —C₁₋₆Alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl, each heterocyclyl and heteroaryl groups at each occurrence independently contain 1, 2, 3 or 4 heteroatoms selected from N, O, S, S═O or S(═O)₂; each R₃ and R₄ at each occurrence is independently and optionally substituted or unsubstituted with 1, 2, 3 or 4, 5 or 6 selected from deuterium, —F, —Cl, —Br, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —Substituents of NR₆C(═O)R₇ or —S(O)₂NR₆R₇.
Preferably, R₆ and R₇ in each R₅ at each occurrence are independently selected from hydrogen, deuterium, or —C₁₋₃ alkyl, or R₆ and R₇ in R₅ together with the N atom to which they are attached together form a 3-6 membered heterocycle, the 3-6 membered heterocycle may further comprise 1 heteroatom selected from N, and the 3-6 membered heterocycle heterocycles are independently and optionally contain 1, 2, 3, 4 heteroatoms selected from N, O or S;
Preferably, each R₅ at each occurrence is independently selected from deuterium, —F, —Cl, —Br, methyl, ethyl, propyl, isopropyl, vinyl, propenyl, isopropyl propenyl, ethynyl, propynyl, -methylene-(halogen)₁₋₃, -ethylene-(halogen)₁₋₃, -propylene-(halogen)₁₋₃, heteromethyl, Heteroethyl, heteropropyl, vinyl, propenyl, ethynyl, propynyl, oxo, —OR₆, -methylene-(OR₆)₁₋₃, -ethylene-(OR₆)₁₋₃, -propylene-(OR₆)₁₋₃, —O-methylene-(halogen)₁₋₃, —O-ethylene-(halogen)₁₋₃, —O-propylene-(halogen))₁₋₃, —NR₆R₇, -methylene-NR₆R₇, -ethylene-NR₆R₇, -propylene-NR₆R₇, —CN, —C(═O)R₆, —C(═O)OR₆, —OC(═O)R₆, —C(═O)NR₆R₇, —NR₆C(═O)R₇, —S(O)₂NR₆R₇, phenyl, naphthyl, 5-membered heteroaryl, 6-membered heteroaryl, 7-membered heteroaryl Aryl, 6-membered heteroaryl, 8-membered heteroaryl, 10-membered heteroaryl, 3-membered heterocyclyl, 4-membered heterocyclyl, 5-membered heterocyclyl, 6-membered heterocyclyl, 3-membered carbocyclyl , 4-membered carbocyclyl, 5-membered carbocyclyl or 6-membered carbocyclyl, each heterocyclyl and heteroaryl at each occurrence independently contain 1, 2, 3 or 4 heteroatoms selected from N, O or S; each R₇ at each occurrence is independently and optionally substituted or unsubstituted with 1, 2, 3, 4, 5 or 6 substituents selected from —F, —Cl, —Br, oxo, methyl, ethyl, propyl, isopropyl, —OR₆, —NR₆R₇, —CN, —C(═O)R₆, —C(═O)OR₆, —OC(═O)R₆, —C(═O)NR₆R₇, —NR₆C(═O)R₆, or —S(O)₂NR₆R₇;
Preferably, R₆ and R₇ in each R₅ at each occurrence are independently selected from hydrogen, deuterium, methyl, ethyl, propyl, isopropyl; or R₆ and R₇ in each R₅ together with the N atom to which they are attached together form 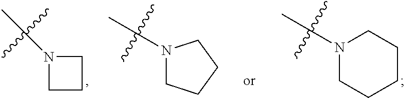 [SMILES: *N1CCC1 ; *N1CCCC1 ; *N1CCCCC1]
Preferably, each R₅ is independently selected at each occurrence from deuterium, —F, —Cl, —Br, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH₂CH₂OCH₃, CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(=O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocyclyl, 5-membered carbocyclyl, or 6-membered Carbocyclyl; each R₂₀ is independently and optionally substituted or unsubstituted 1, 2, 3, 4, 5 or 6 substituents selected from -deuterium, —F, —Cl, —Br, methyl, ethyl, propyl, isopropyl base, methoxy, ethoxy, propoxy, isopropoxy, oxo, —OH, —NH₂, —NHCH₃, _N(CH₃)₂, —CN, —C(═O)CH₃, _C(═O)OO, —OC(═O)O, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂;

6. The compound of formula (I), a pharmaceutically acceptable salt thereof, or a stereoisomer thereof according to claim 1, wherein L₁-R₁₉ are selected from the following structures:

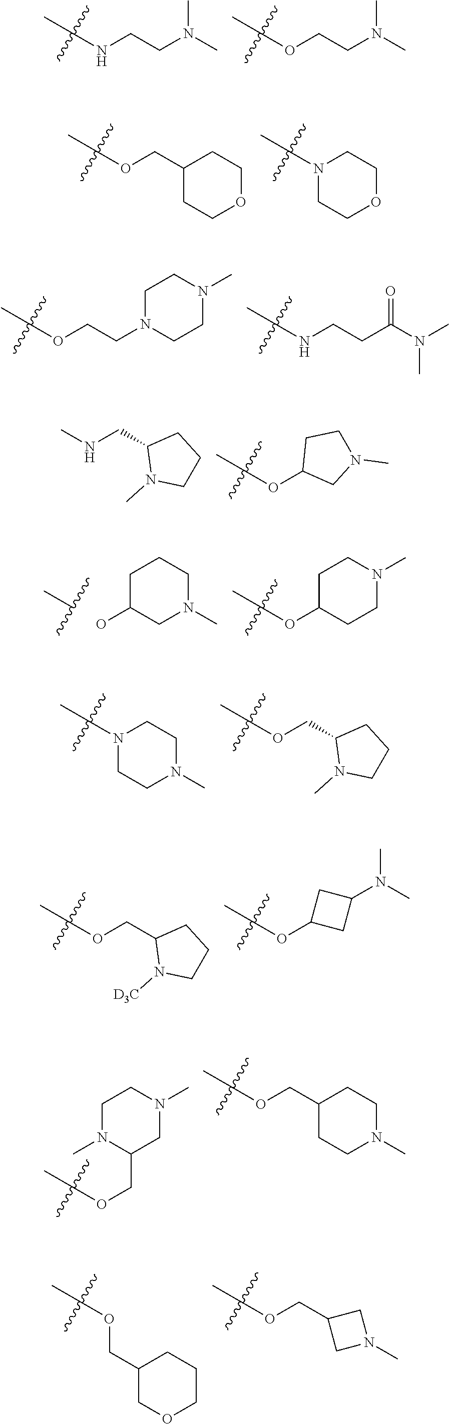

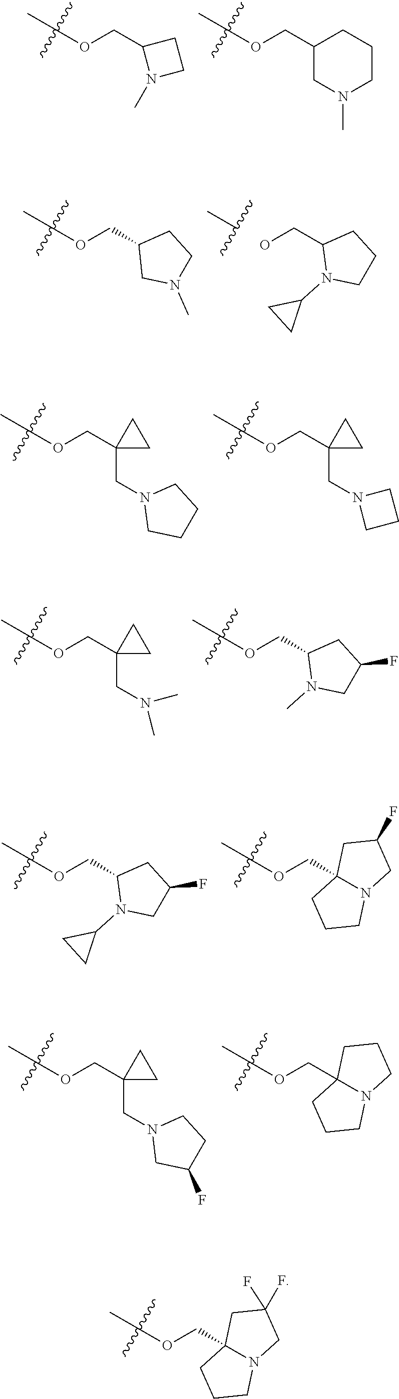

7. The compound of formula (I) according to claim 1, its pharmaceutically acceptable salt or its stereoisomer, wherein, the compound is selected from the following structures:

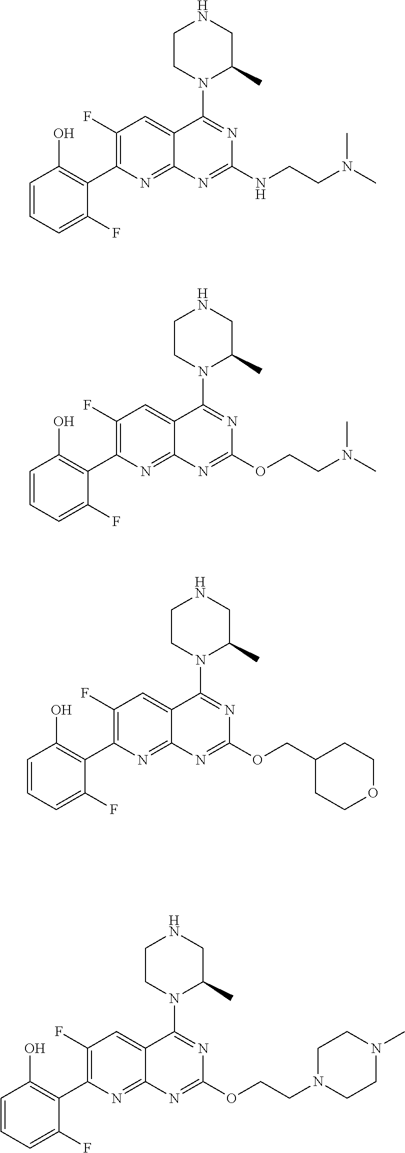 [SMILES: C[C@@H]1CNCCN1c1nc(NCCN(C)C)nc2nc(-c3c(O)cccc3F)c(F)cc12 ; C[C@@H]1CNCCN1c1nc(OCCN(C)C)nc2nc(-c3c(O)cccc3F)c(F)cc12 ; C[C@@H]1CNCCN1c1nc(OCC2CCOCC2)nc2nc(-c3c(O)cccc3F)c(F)cc12 ; C[C@@H]1CNCCN1c1nc(OCCN2CCN(C)CC2)nc2nc(-c3c(O)cccc3F)c(F)cc12]

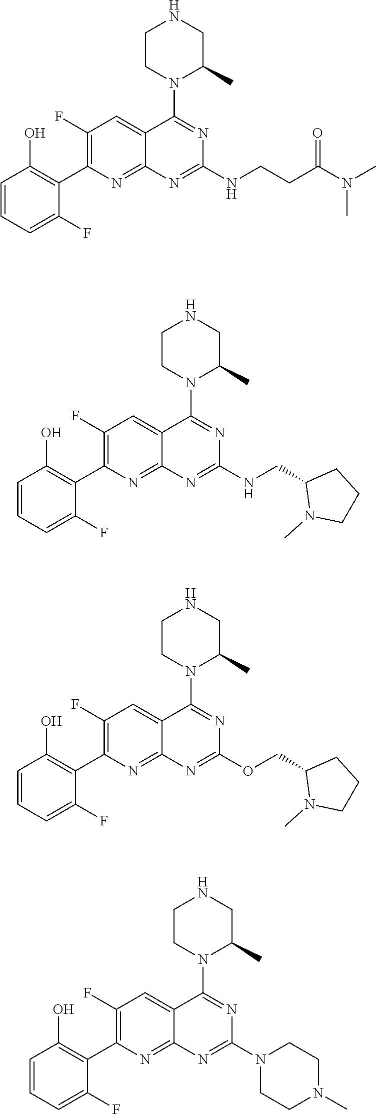 [SMILES: C[C@@H]1CNCCN1c1nc(NCCC(=O)N(C)C)nc2nc(-c3c(O)cccc3F)c(F)cc12 ; C[C@@H]1CNCCN1c1nc(NC[C@@H]2CCCN2C)nc2nc(-c3c(O)cccc3F)c(F)cc12 ; C[C@@H]1CNCCN1c1nc(OC[C@@H]2CCCN2C)nc2nc(-c3c(O)cccc3F)c(F)cc12 ; C[C@@H]1CNCCN1c1nc(N2CCN(C)CC2)nc2nc(-c3c(O)cccc3F)c(F)cc12]

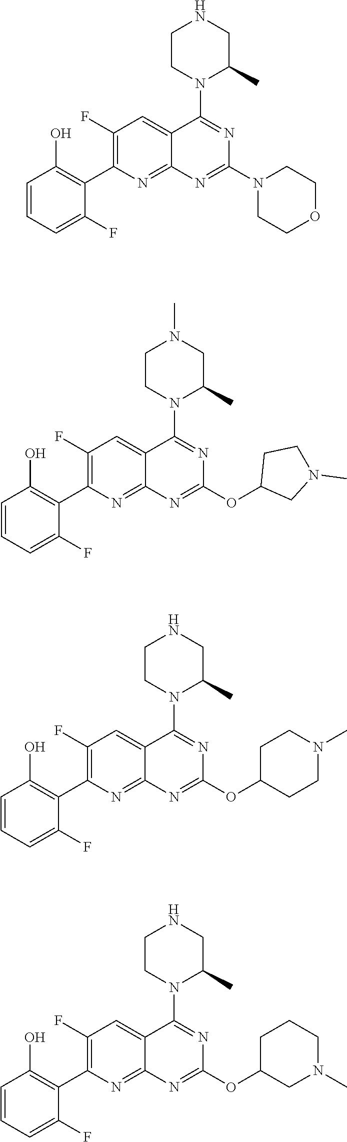 [SMILES: C[C@@H]1CNCCN1c1nc(N2CCOCC2)nc2nc(-c3c(O)cccc3F)c(F)cc12 ; C[C@@H]1CN(C)CCN1c1nc(OC2CCN(C)C2)nc2nc(-c3c(O)cccc3F)c(F)cc12 ; C[C@@H]1CNCCN1c1nc(OC2CCN(C)CC2)nc2nc(-c3c(O)cccc3F)c(F)cc12 ; C[C@@H]1CNCCN1c1nc(OC2CCCN(C)C2)nc2nc(-c3c(O)cccc3F)c(F)cc12]

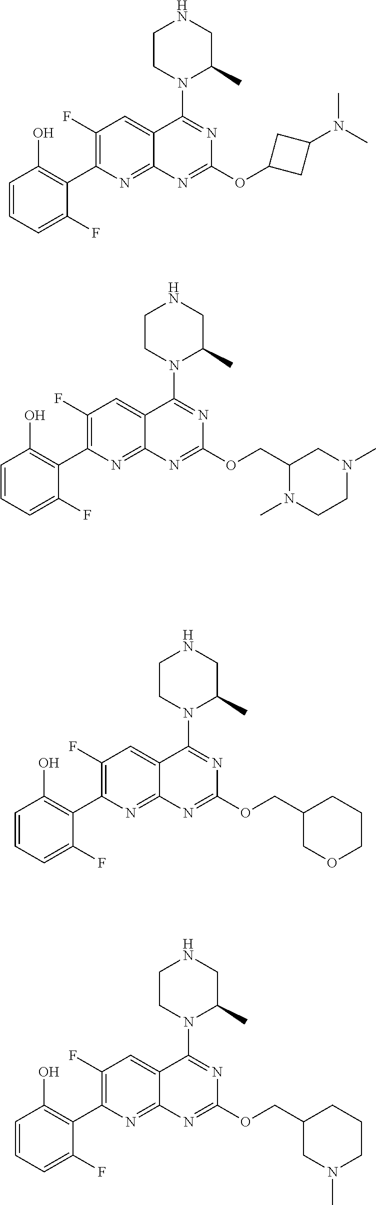 [SMILES: C[C@@H]1CNCCN1c1nc(OC2CC(N(C)C)C2)nc2nc(-c3c(O)cccc3F)c(F)cc12 ; C[C@@H]1CNCCN1c1nc(OCC2CN(C)CCN2C)nc2nc(-c3c(O)cccc3F)c(F)cc12 ; C[C@@H]1CNCCN1c1nc(OCC2CCCOC2)nc2nc(-c3c(O)cccc3F)c(F)cc12 ; C[C@@H]1CNCCN1c1nc(OCC2CCCN(C)C2)nc2nc(-c3c(O)cccc3F)c(F)cc12]

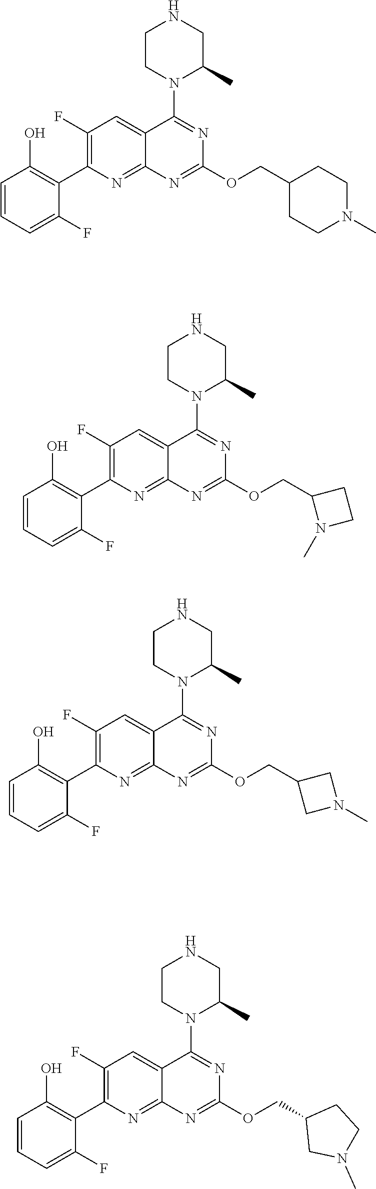 [SMILES: C[C@@H]1CNCCN1c1nc(OCC2CCN(C)CC2)nc2nc(-c3c(O)cccc3F)c(F)cc12 ; C[C@@H]1CNCCN1c1nc(OCC2CCN2C)nc2nc(-c3c(O)cccc3F)c(F)cc12 ; C[C@@H]1CNCCN1c1nc(OCC2CN(C)C2)nc2nc(-c3c(O)cccc3F)c(F)cc12 ; C[C@@H]1CNCCN1c1nc(OC[C@@H]2CCN(C)C2)nc2nc(-c3c(O)cccc3F)c(F)cc12]

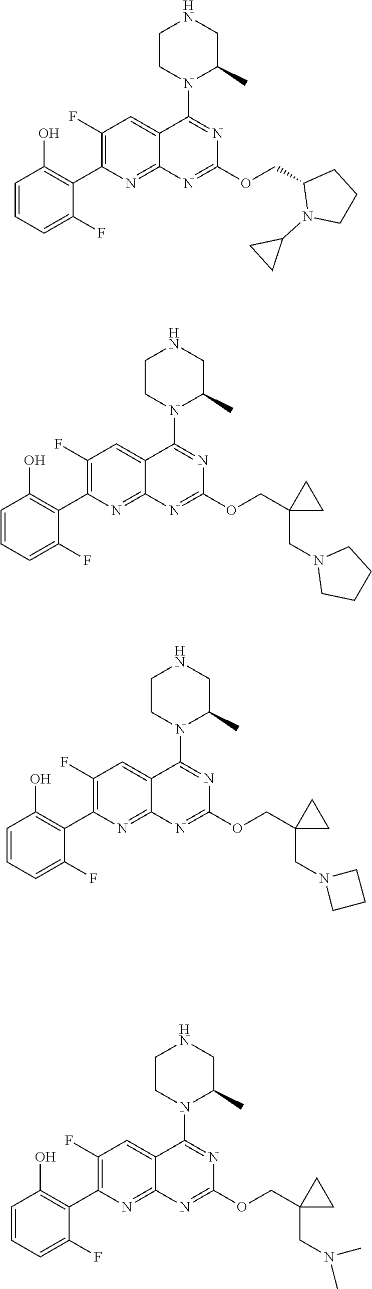 [SMILES: C[C@@H]1CNCCN1c1nc(OC[C@@H]2CCCN2C2CC2)nc2nc(-c3c(O)cccc3F)c(F)cc12 ; C[C@@H]1CNCCN1c1nc(OCC2(CN3CCCC3)CC2)nc2nc(-c3c(O)cccc3F)c(F)cc12 ; C[C@@H]1CNCCN1c1nc(OCC2(CN3CCC3)CC2)nc2nc(-c3c(O)cccc3F)c(F)cc12 ; C[C@@H]1CNCCN1c1nc(OCC2(CN(C)C)CC2)nc2nc(-c3c(O)cccc3F)c(F)cc12]

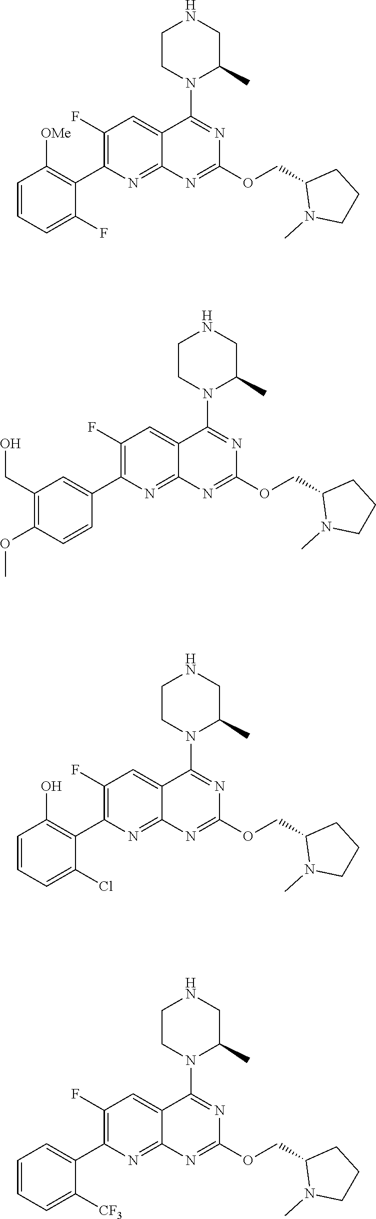 [SMILES: COc1cccc(F)c1-c1nc2nc(OC[C@@H]3CCCN3C)nc(N3CCNC[C@H]3C)c2cc1F ; COc1ccc(-c2nc3nc(OC[C@@H]4CCCN4C)nc(N4CCNC[C@H]4C)c3cc2F)cc1CO ; C[C@@H]1CNCCN1c1nc(OC[C@@H]2CCCN2C)nc2nc(-c3c(O)cccc3Cl)c(F)cc12 ; C[C@@H]1CNCCN1c1nc(OC[C@@H]2CCCN2C)nc2nc(-c3ccccc3C(F)(F)F)c(F)cc12]

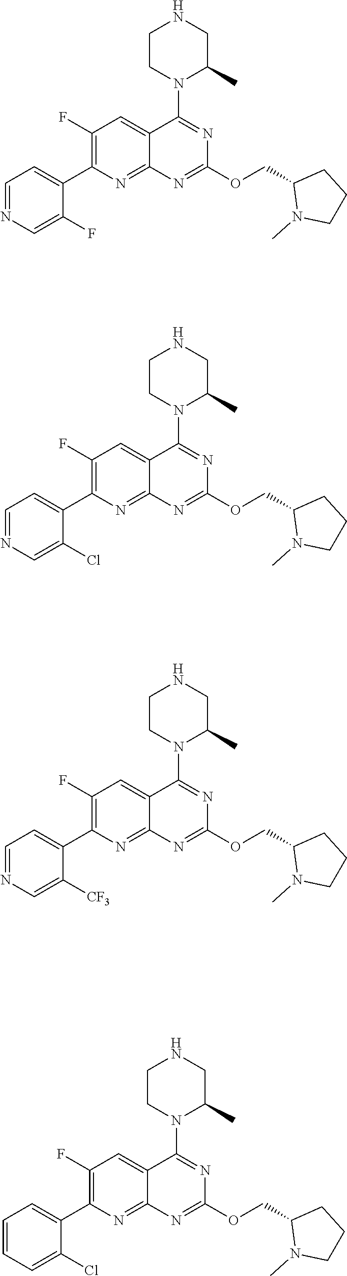 [SMILES: C[C@@H]1CNCCN1c1nc(OC[C@@H]2CCCN2C)nc2nc(-c3ccncc3F)c(F)cc12 ; C[C@@H]1CNCCN1c1nc(OC[C@@H]2CCCN2C)nc2nc(-c3ccncc3Cl)c(F)cc12 ; C[C@@H]1CNCCN1c1nc(OC[C@@H]2CCCN2C)nc2nc(-c3ccncc3C(F)(F)F)c(F)cc12 ; C[C@@H]1CNCCN1c1nc(OC[C@@H]2CCCN2C)nc2nc(-c3ccccc3Cl)c(F)cc12]

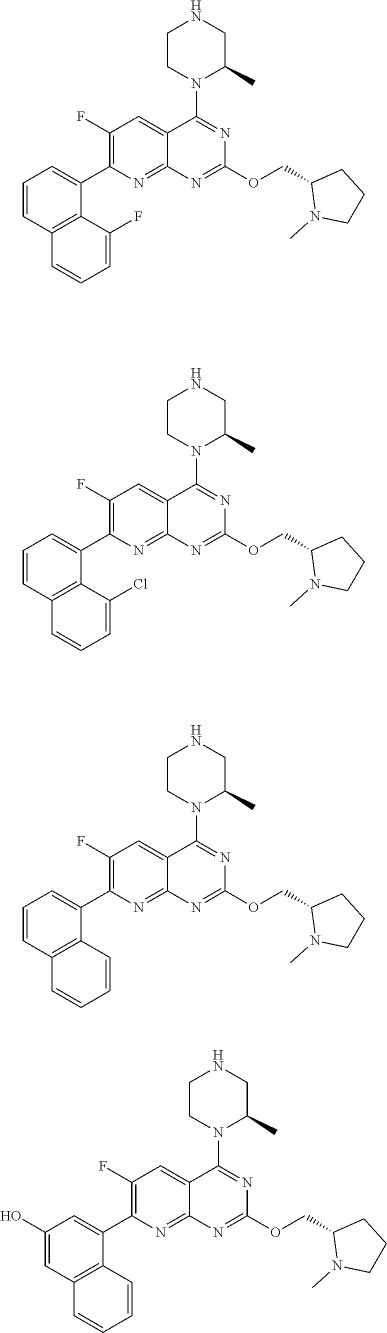 [SMILES: C[C@@H]1CNCCN1c1nc(OC[C@@H]2CCCN2C)nc2nc(-c3cccc4cccc(F)c34)c(F)cc12 ; C[C@@H]1CNCCN1c1nc(OC[C@@H]2CCCN2C)nc2nc(-c3cccc4cccc(Cl)c34)c(F)cc12 ; C[C@@H]1CNCCN1c1nc(OC[C@@H]2CCCN2C)nc2nc(-c3cccc4ccccc34)c(F)cc12 ; C[C@@H]1CNCCN1c1nc(OC[C@@H]2CCCN2C)nc2nc(-c3cc(O)cc4ccccc34)c(F)cc12]

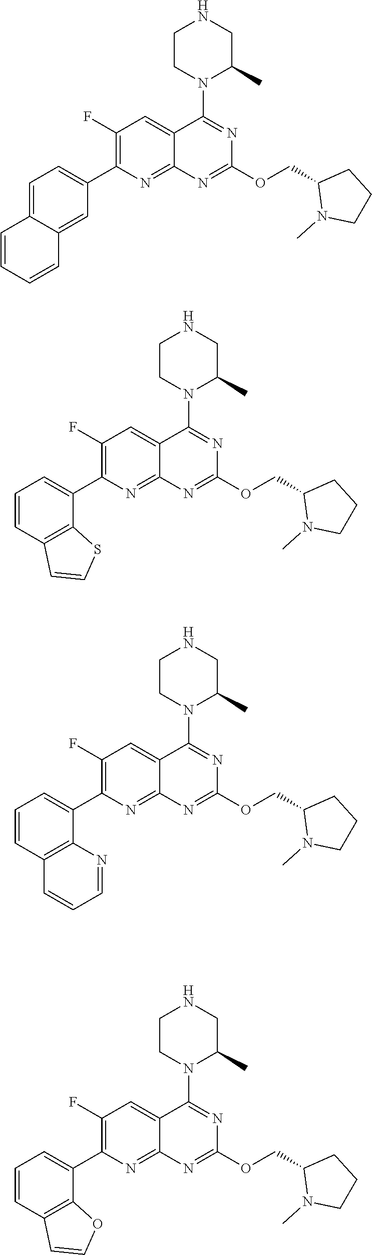 [SMILES: C[C@@H]1CNCCN1c1nc(OC[C@@H]2CCCN2C)nc2nc(-c3ccc4ccccc4c3)c(F)cc12 ; C[C@@H]1CNCCN1c1nc(OC[C@@H]2CCCN2C)nc2nc(-c3cccc4ccsc34)c(F)cc12 ; C[C@@H]1CNCCN1c1nc(OC[C@@H]2CCCN2C)nc2nc(-c3cccc4cccnc34)c(F)cc12 ; C[C@@H]1CNCCN1c1nc(OC[C@@H]2CCCN2C)nc2nc(-c3cccc4ccoc34)c(F)cc12]

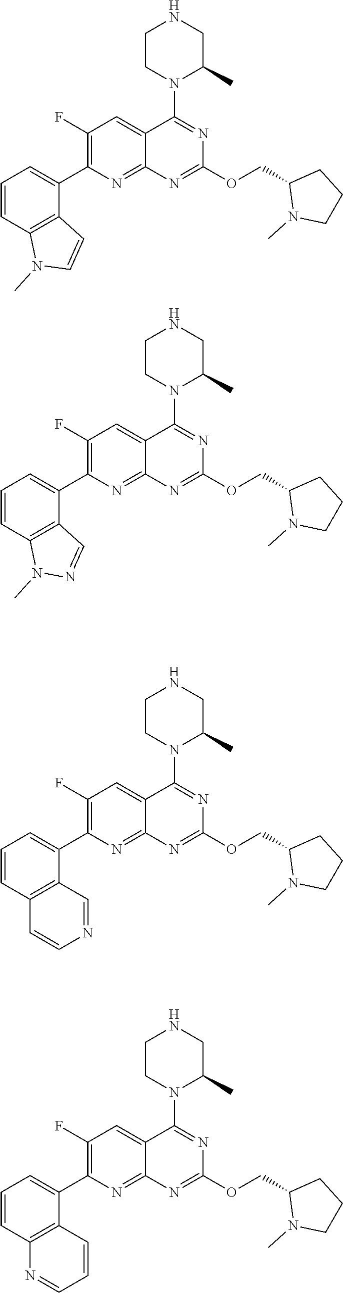 [SMILES: C[C@@H]1CNCCN1c1nc(OC[C@@H]2CCCN2C)nc2nc(-c3cccc4c3ccn4C)c(F)cc12 ; C[C@@H]1CNCCN1c1nc(OC[C@@H]2CCCN2C)nc2nc(-c3cccc4c3cnn4C)c(F)cc12 ; C[C@@H]1CNCCN1c1nc(OC[C@@H]2CCCN2C)nc2nc(-c3cccc4ccncc34)c(F)cc12 ; C[C@@H]1CNCCN1c1nc(OC[C@@H]2CCCN2C)nc2nc(-c3cccc4ncccc34)c(F)cc12]

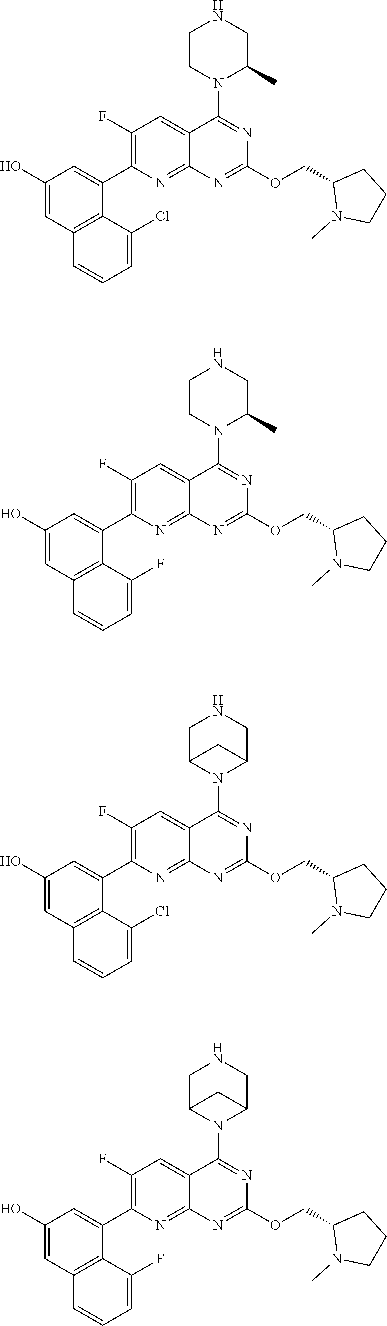 [SMILES: C[C@@H]1CNCCN1c1nc(OC[C@@H]2CCCN2C)nc2nc(-c3cc(O)cc4cccc(Cl)c34)c(F)cc12 ; C[C@@H]1CNCCN1c1nc(OC[C@@H]2CCCN2C)nc2nc(-c3cc(O)cc4cccc(F)c34)c(F)cc12 ; CN1CCC[C@H]1COc1nc(N2C3CNCC2C3)c2cc(F)c(-c3cc(O)cc4cccc(Cl)c34)nc2n1 ; CN1CCC[C@H]1COc1nc(N2C3CNCC2C3)c2cc(F)c(-c3cc(O)cc4cccc(F)c34)nc2n1]

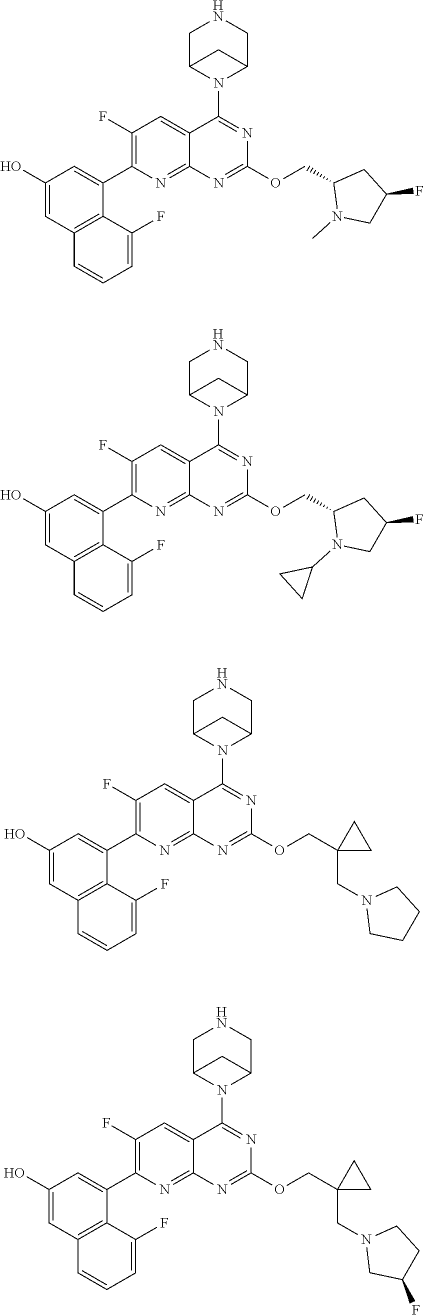 [SMILES: CN1C[C@H](F)C[C@H]1COc1nc(N2C3CNCC2C3)c2cc(F)c(-c3cc(O)cc4cccc(F)c34)nc2n1 ; Oc1cc(-c2nc3nc(OC[C@@H]4C[C@@H](F)CN4C4CC4)nc(N4C5CNCC4C5)c3cc2F)c2c(F)cccc2c1 ; Oc1cc(-c2nc3nc(OCC4(CN5CCCC5)CC4)nc(N4C5CNCC4C5)c3cc2F)c2c(F)cccc2c1 ; Oc1cc(-c2nc3nc(OCC4(CN5CC[C@@H](F)C5)CC4)nc(N4C5CNCC4C5)c3cc2F)c2c(F)cccc2c1]

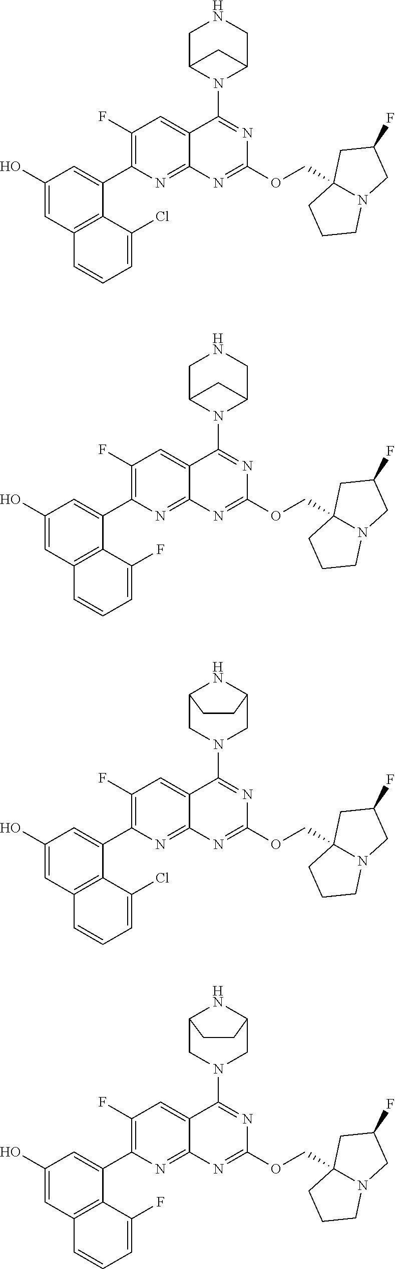 [SMILES: Oc1cc(-c2nc3nc(OC[C@@]45CCCN4C[C@H](F)C5)nc(N4C5CNCC4C5)c3cc2F)c2c(Cl)cccc2c1 ; Oc1cc(-c2nc3nc(OC[C@@]45CCCN4C[C@H](F)C5)nc(N4C5CNCC4C5)c3cc2F)c2c(F)cccc2c1 ; Oc1cc(-c2nc3nc(OC[C@@]45CCCN4C[C@H](F)C5)nc(N4CC5CCC(C4)N5)c3cc2F)c2c(Cl)cccc2c1 ; Oc1cc(-c2nc3nc(OC[C@@]45CCCN4C[C@H](F)C5)nc(N4CC5CCC(C4)N5)c3cc2F)c2c(F)cccc2c1]

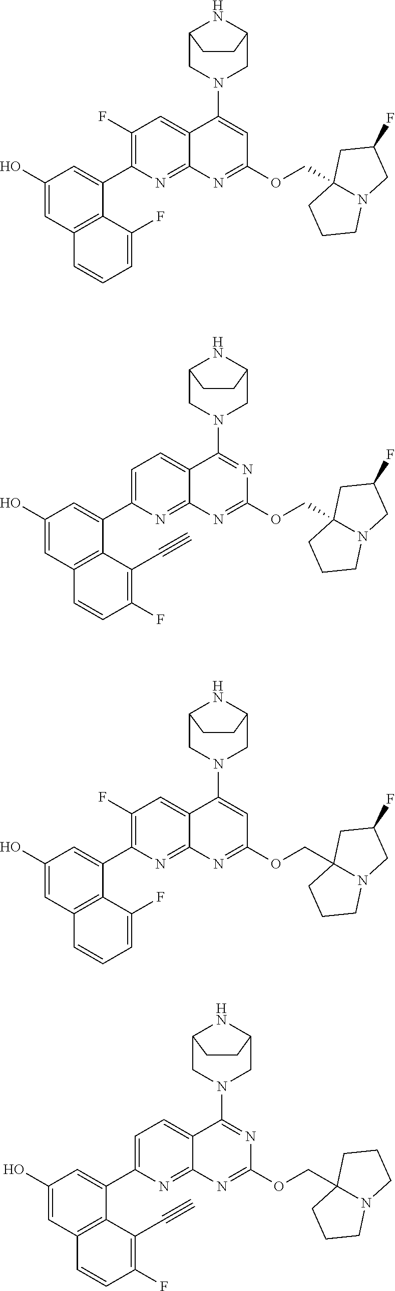 [SMILES: Oc1cc(-c2nc3nc(OC[C@@]45CCCN4C[C@H](F)C5)cc(N4CC5CCC(C4)N5)c3cc2F)c2c(F)cccc2c1 ; C#Cc1c(F)ccc2cc(O)cc(-c3ccc4c(N5CC6CCC(C5)N6)nc(OC[C@@]56CCCN5C[C@H](F)C6)nc4n3)c12 ; Oc1cc(-c2nc3nc(OCC45CCCN4C[C@H](F)C5)cc(N4CC5CCC(C4)N5)c3cc2F)c2c(F)cccc2c1 ; C#Cc1c(F)ccc2cc(O)cc(-c3ccc4c(N5CC6CCC(C5)N6)nc(OCC56CCCN5CCC6)nc4n3)c12]

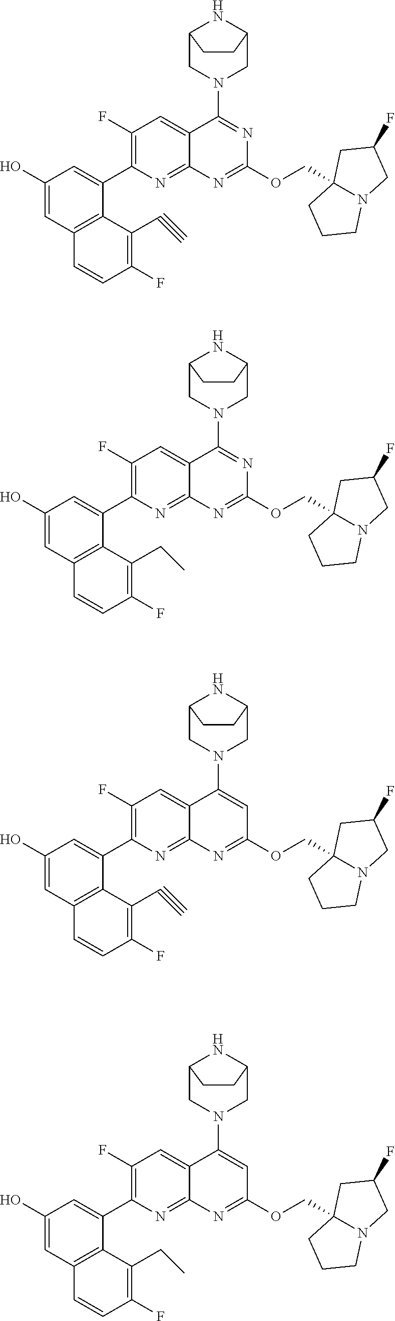 [SMILES: C#Cc1c(F)ccc2cc(O)cc(-c3nc4nc(OC[C@@]56CCCN5C[C@H](F)C6)nc(N5CC6CCC(C5)N6)c4cc3F)c12 ; CCc1c(F)ccc2cc(O)cc(-c3nc4nc(OC[C@@]56CCCN5C[C@H](F)C6)nc(N5CC6CCC(C5)N6)c4cc3F)c12 ; C#Cc1c(F)ccc2cc(O)cc(-c3nc4nc(OC[C@@]56CCCN5C[C@H](F)C6)cc(N5CC6CCC(C5)N6)c4cc3F)c12 ; CCc1c(F)ccc2cc(O)cc(-c3nc4nc(OC[C@@]56CCCN5C[C@H](F)C6)cc(N5CC6CCC(C5)N6)c4cc3F)c12]

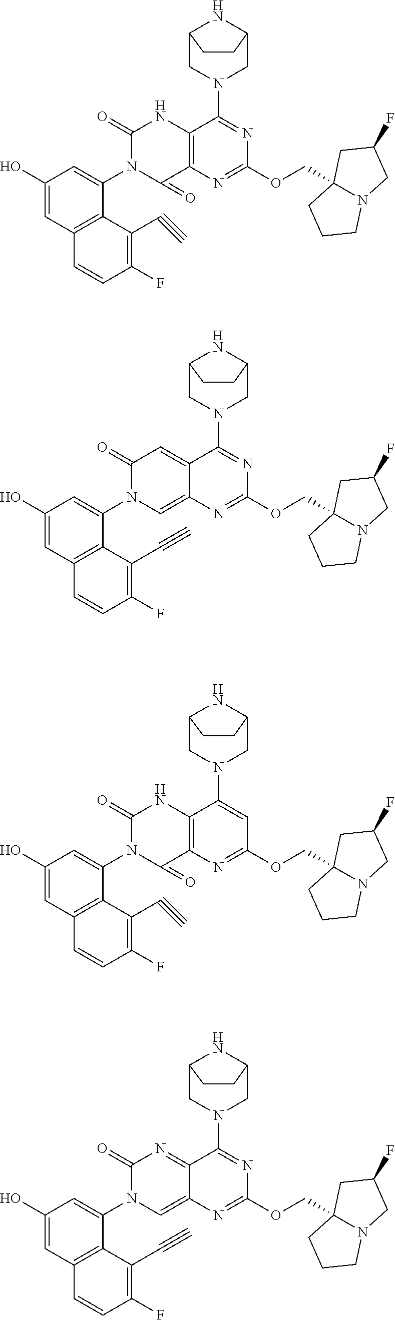 [SMILES: C#Cc1c(F)ccc2cc(O)cc(-n3c(=O)[nH]c4c(N5CC6CCC(C5)N6)nc(OC[C@@]56CCCN5C[C@H](F)C6)nc4c3=O)c12 ; C#Cc1c(F)ccc2cc(O)cc(-n3cc4nc(OC[C@@]56CCCN5C[C@H](F)C6)nc(N5CC6CCC(C5)N6)c4cc3=O)c12 ; C#Cc1c(F)ccc2cc(O)cc(-n3c(=O)[nH]c4c(N5CC6CCC(C5)N6)cc(OC[C@@]56CCCN5C[C@H](F)C6)nc4c3=O)c12 ; C#Cc1c(F)ccc2cc(O)cc(-n3cc4nc(OC[C@@]56CCCN5C[C@H](F)C6)nc(N5CC6CCC(C5)N6)c4nc3=O)c12]

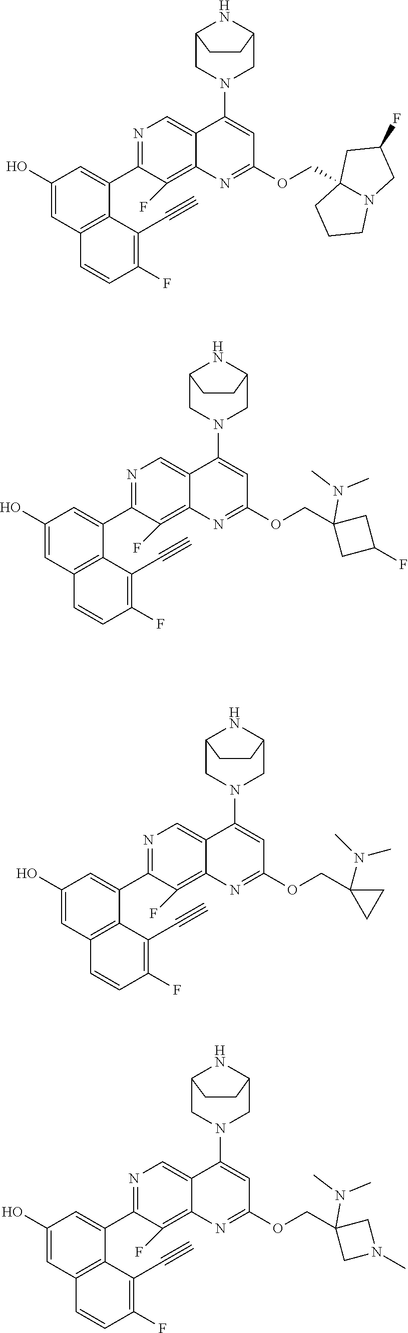 [SMILES: C#Cc1c(F)ccc2cc(O)cc(-c3ncc4c(N5CC6CCC(C5)N6)cc(OC[C@@]56CCCN5C[C@H](F)C6)nc4c3F)c12 ; C#Cc1c(F)ccc2cc(O)cc(-c3ncc4c(N5CC6CCC(C5)N6)cc(OCC5(N(C)C)CC(F)C5)nc4c3F)c12 ; C#Cc1c(F)ccc2cc(O)cc(-c3ncc4c(N5CC6CCC(C5)N6)cc(OCC5(N(C)C)CC5)nc4c3F)c12 ; C#Cc1c(F)ccc2cc(O)cc(-c3ncc4c(N5CC6CCC(C5)N6)cc(OCC5(N(C)C)CN(C)C5)nc4c3F)c12]

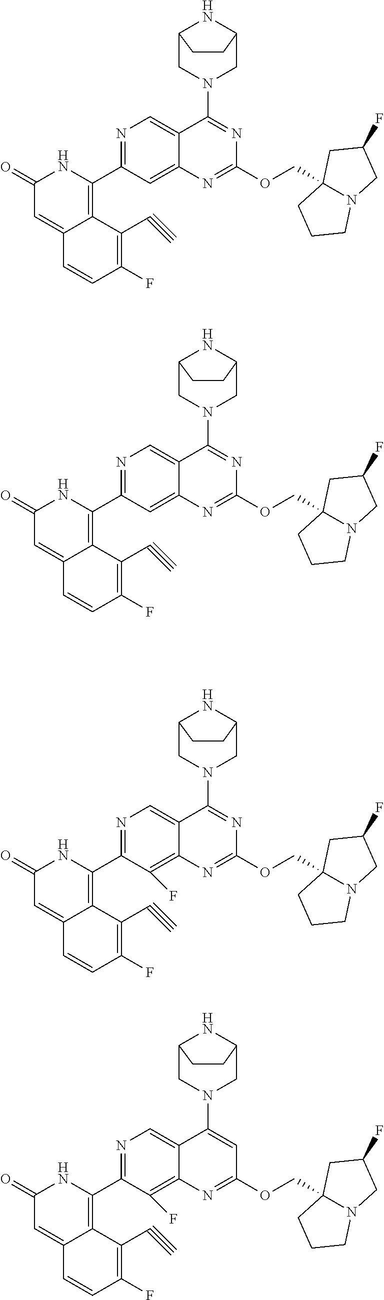 [SMILES: C#Cc1c(F)ccc2cc(=O)[nH]c(-c3cc4nc(OC[C@@]56CCCN5C[C@H](F)C6)nc(N5CC6CCC(C5)N6)c4cn3)c12 ; C#Cc1c(F)ccc2cc(=O)[nH]c(-c3cc4nc(OC[C@@]56CCCN5C[C@H](F)C6)nc(N5CC6CCC(C5)N6)c4cn3)c12 ; C#Cc1c(F)ccc2cc(=O)[nH]c(-c3ncc4c(N5CC6CCC(C5)N6)nc(OC[C@@]56CCCN5C[C@H](F)C6)nc4c3F)c12 ; C#Cc1c(F)ccc2cc(=O)[nH]c(-c3ncc4c(N5CC6CCC(C5)N6)cc(OC[C@@]56CCCN5C[C@H](F)C6)nc4c3F)c12]

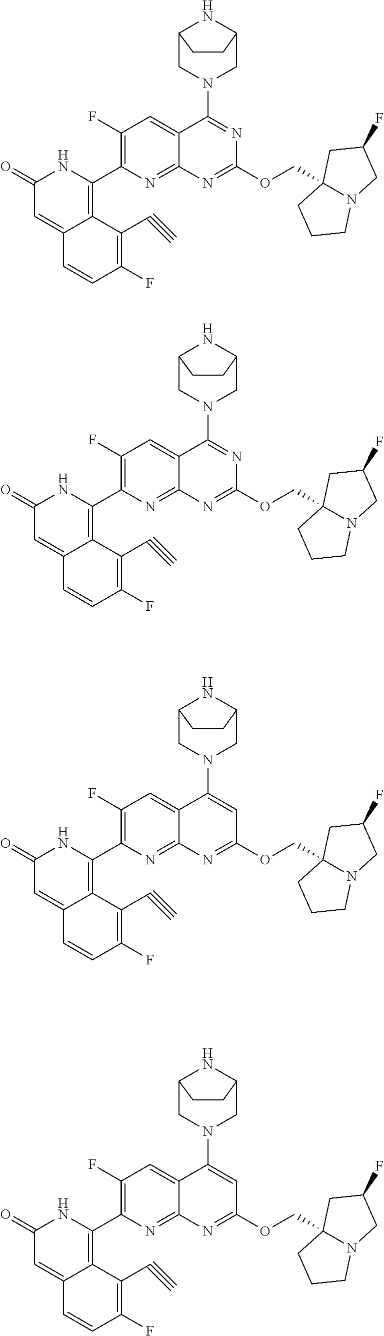 [SMILES: C#Cc1c(F)ccc2cc(=O)[nH]c(-c3nc4nc(OC[C@@]56CCCN5C[C@H](F)C6)nc(N5CC6CCC(C5)N6)c4cc3F)c12 ; C#Cc1c(F)ccc2cc(=O)[nH]c(-c3nc4nc(OC[C@@]56CCCN5C[C@H](F)C6)nc(N5CC6CCC(C5)N6)c4cc3F)c12 ; C#Cc1c(F)ccc2cc(=O)[nH]c(-c3nc4nc(OC[C@@]56CCCN5C[C@H](F)C6)cc(N5CC6CCC(C5)N6)c4cc3F)c12 ; C#Cc1c(F)ccc2cc(=O)[nH]c(-c3nc4nc(OC[C@@]56CCCN5C[C@H](F)C6)cc(N5CC6CCC(C5)N6)c4cc3F)c12]

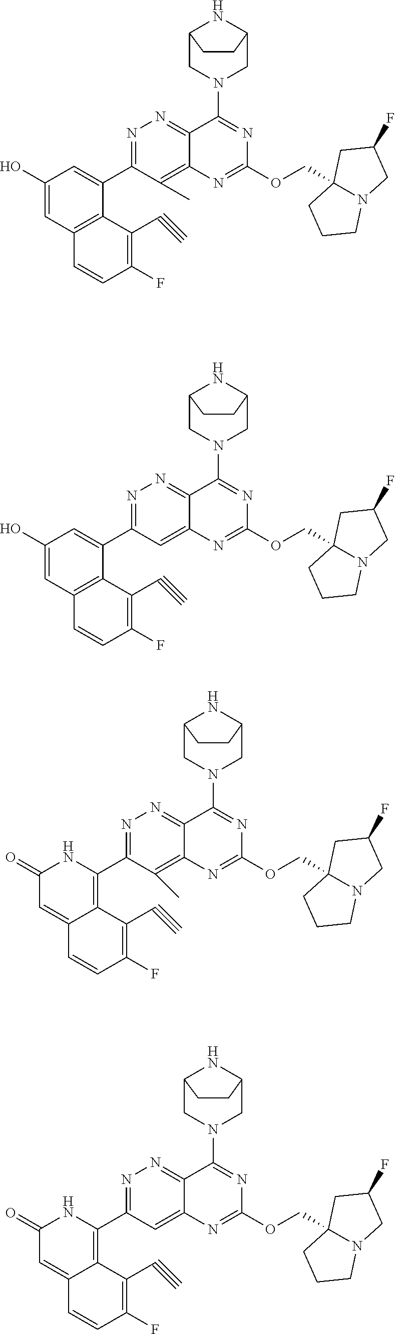 [SMILES: C#Cc1c(F)ccc2cc(O)cc(-c3nnc4c(N5CC6CCC(C5)N6)nc(OC[C@@]56CCCN5C[C@H](F)C6)nc4c3C)c12 ; C#Cc1c(F)ccc2cc(O)cc(-c3cc4nc(OC[C@@]56CCCN5C[C@H](F)C6)nc(N5CC6CCC(C5)N6)c4nn3)c12 ; C#Cc1c(F)ccc2cc(=O)[nH]c(-c3nnc4c(N5CC6CCC(C5)N6)nc(OC[C@@]56CCCN5C[C@H](F)C6)nc4c3C)c12 ; C#Cc1c(F)ccc2cc(=O)[nH]c(-c3cc4nc(OC[C@@]56CCCN5C[C@H](F)C6)nc(N5CC6CCC(C5)N6)c4nn3)c12]

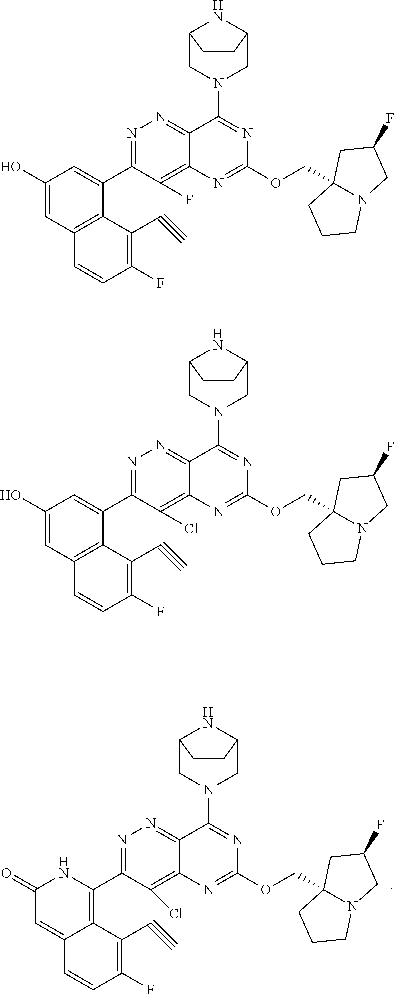 [SMILES: C#Cc1c(F)ccc2cc(O)cc(-c3nnc4c(N5CC6CCC(C5)N6)nc(OC[C@@]56CCCN5C[C@H](F)C6)nc4c3F)c12 ; C#Cc1c(F)ccc2cc(O)cc(-c3nnc4c(N5CC6CCC(C5)N6)nc(OC[C@@]56CCCN5C[C@H](F)C6)nc4c3Cl)c12 ; C#Cc1c(F)ccc2cc(=O)[nH]c(-c3nnc4c(N5CC6CCC(C5)N6)nc(OC[C@@]56CCCN5C[C@H](F)C6)nc4c3Cl)c12]

8. A pharmaceutical composition comprising the compound according to claim 1 or a pharmaceutically acceptable salt of said compound, and a pharmaceutically acceptable excipient.

9. The use of a compound as claimed in claim 1, characterized in that it is used for preparing a pharmaceutical composition for: (i) preventing and/or treating tumors; (ii) Inhibiting or reversing the multidrug resistance of tumors to anti-tumor drugs; (iii) Inhibition of P-glycoprotein; (iv) Enhance the anti-tumor activity of anti-tumor drugs; The application of drugs to inhibit cancer associated with KRASG12D mutant protein and/or (v). Preferably, the cancer is selected from the following groups: blood cancer, lung cancer, pancreatic cancer, colon cancer, rectal cancer, colorectal cancer, and Oral cancer; The blood cancer is selected from acute myeloid leukemia or acute lymphocytic leukemia, and the lung cancer is selected from non-small cell lung cancer or small cell lung cancer.

10. The use as claimed in claim 9, characterized in that the tumor includes tumors that develop multidrug resistance to anti-tumor drugs.
